SEQUENCE LISTING (4 sequences)

SEQ ID NO: 1 - DNA, 20 bp, synthetic construct
  source 1..20: mol_type = other DNA
tggtttccggcaagcttcaa

SEQ ID NO: 2 - DNA, 20 bp, synthetic construct
  source 1..20: mol_type = other DNA
tgttgtcgatacccttgggc

SEQ ID NO: 3 - DNA, 22 bp, synthetic construct
  source 1..22: mol_type = other DNA
tcgctatctggcaatactgctg

SEQ ID NO: 4 - DNA, 19 bp, synthetic construct
  source 1..19: mol_type = other DNA
gttagggcggccgttgact

(19) United States
(12) Patent Application Publication
(10) Pub. No.: US 2025/0099482 A1
(43) Pub. Date: Mar. 27, 2025

(54) TIP60 NEURO-EPIGENETIC MODULATORS

(71) Applicant: Drexel University, Philadelphia, PA (US)

(72) Inventors: Sandhya Kortagere, Newtown, PA (US); Felice Elefant, Philadelphia, PA (US); Akanksha Bhatnagar, Philadelphia, PA (US)

(21) Appl. No.: 18/891,352

(22) Filed: Sep. 20, 2024

Related U.S. Application Data
(60) Provisional application No. 63/539,914, filed on Sep. 22, 2023.

(51) Int. Cl. A61K 31/551; A61K 31/136; A61K 31/167; A61K 31/341; A61K 31/381; A61K 31/415; A61K 31/4196; A61K 31/437; A61K 31/444; A61K 31/47; A61K 31/4985; A61K 31/5375; A61P 25/28

(52) U.S. Cl. CPC A61K 31/551; A61K 31/136; A61K 31/167; A61K 31/341; A61K 31/381; A61K 31/415; A61K 31/4196; A61K 31/437; A61K 31/444; A61K 31/47; A61K 31/4985; A61K 31/5375; A61P 25/28

(57) ABSTRACT

The present disclosure relates, in part, to compounds, compositions, and methods for treatment, ameliorating, and/or prevention of certain neurodegenerative diseases and/or cancers.

### CROSS-REFERENCE TO RELATED APPLICATIONS

This application claims priority under 35 U.S.C. § 119 (e) to U.S. Provisional Patent Application No. 63/539,914, filed Sep. 22, 2023, which application is incorporated herein by reference in its entirety.

### STATEMENT REGARDING FEDERALLY SPONSORED RESEARCH OR DEVELOPMENT

This invention was made with government support under NS095799-06 awarded by the National Institutes of Health. The government has certain rights in the invention.

### REFERENCE TO AN ELECTRONIC SEQUENCE LISTING

The XML file named “046528-7135US1_SequenceListing.xml” created on Sep. 19, 2024, comprising 4.25 Kbytes, is hereby incorporated herein by reference in its entirety.

### BACKGROUND

Reduced neuroepigenetic histone acetylation states in the Alzheimer's Disease (AD) brain is a widespread mechanistic hallmark underlying neural gene repression and accompanying debilitating cognitive impairments that characterize AD etiology. Accordingly, pharmacological treatments aiming to restore histone acetylation via histone deacetylase (HDAC) inhibition are promising in reducing cognitive impairment but are rarely translated into clinical trials due to non-specific global hyperacetylation. Alternatively, enhancing the activity of specific histone acetyltransferases (HATs) like Tip60, that play a non-redundant neuroprotective role in AD pathology, serves as an exciting new therapeutic strategy.

There is thus a need in the art for compounds which modulate the activity of HATs (e.g., Tip60) and methods of use thereof for the treatment, prevention and/or amelioration of neurodegenerative diseases and/or disorders, including but not limited to Alzheimer's disease, Parkinson's disease, Huntington's disease, and/or amyotrophic lateral sclerosis (ALS). The present disclosure addresses this need.

### BRIEF SUMMARY

In one aspect, the disclosure provides a method of treating, preventing, and/or ameliorating a neurodegenerative disease. In certain embodiments, the method comprises administering to a subject in need thereof a therapeutically effective amount of at least one Tip60 activator or a pharmaceutically acceptable salt thereof.

In another aspect, the disclosure provides a method of treating, preventing, and/or ameliorating a Tip60-dependent and/or Tip60-overexpressing cancer. In certain embodiments, the method comprises administering to a subject in need thereof a therapeutically effective amount of a Tip60 inhibitor or a pharmaceutically acceptable salt thereof.

In certain embodiments, the Tip60 activator is a compound of Formula (I) or a pharmaceutically acceptable salt thereof, wherein R^{1a}, R^{1b}, R^{1c}, R^{1d}, R^{1e}, R², R^{3a}, R^{3b}, R^{3c}, R^{3d}, and R^{3e} are defined elsewhere herein:

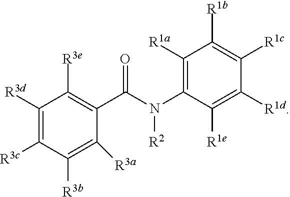

In certain embodiments, the Tip60 activator is a compound of Formula (II) or a pharmaceutically acceptable salt thereof, wherein R⁴, R⁵, R^{6a}, R^{6b}, R⁷, R⁸, L¹, and L² are defined elsewhere herein:

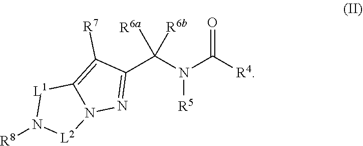

In certain embodiments, the Tip60 activator is selected from the group consisting of:

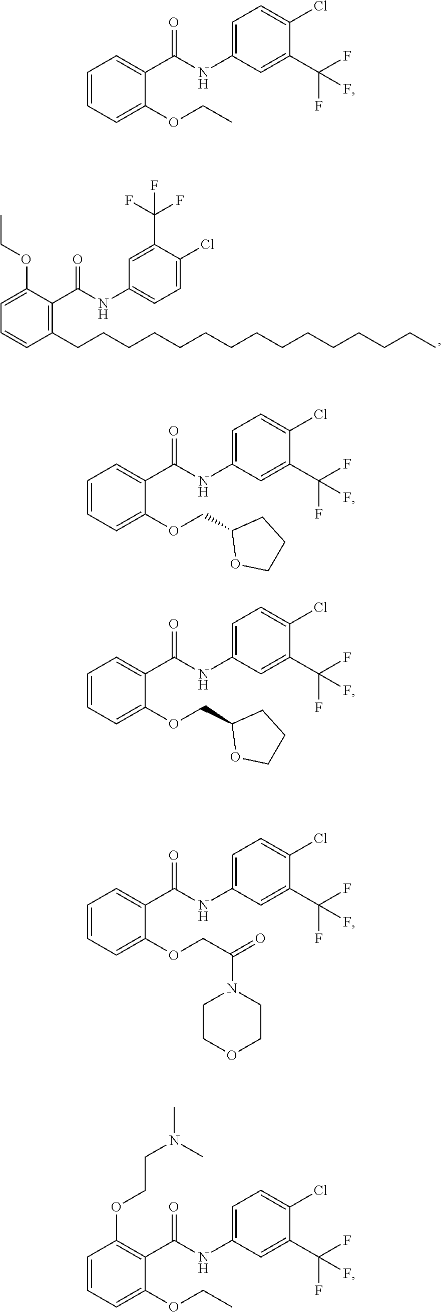

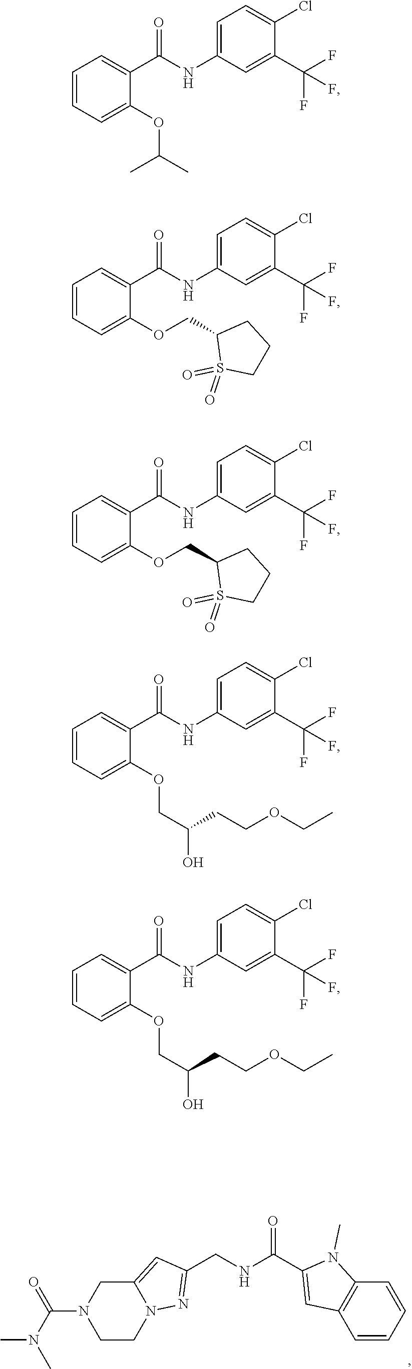

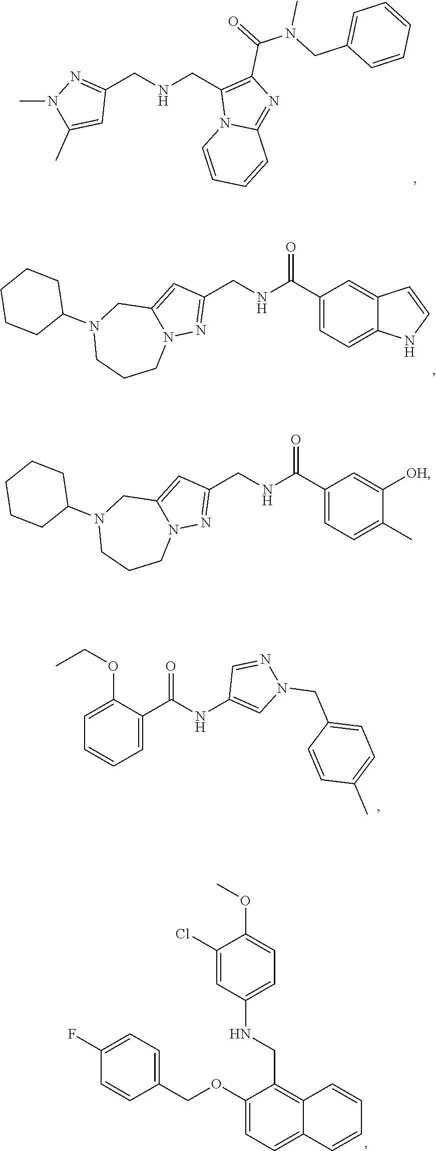

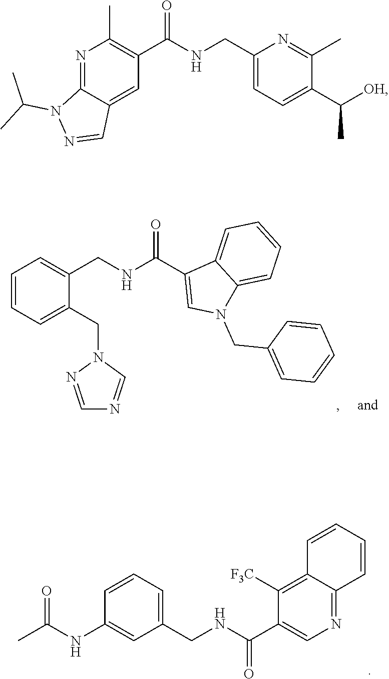 [SMILES: Cc1nc2c(cnn2C(C)C)cc1C(=O)NCc1ccc([C@H](C)O)c(C)n1 ; O=C(NCc1ccccc1Cn1cncn1)c1cn(Cc2ccccc2)c2ccccc12 ; CC(=O)Nc1cccc(CNC(=O)c2cnc3ccccc3c2C(F)(F)F)c1]

### BRIEF DESCRIPTION OF THE FIGURES

The drawings illustrate generally, by way of example, but not by way of limitation, various embodiments of the present application.

FIGS. 1A-1B: Tip60 promotes neural gene expression via histone acetylation function. FIG. 1A: Structural similarity between Drosophila Tip60 (Uniprot: Q960X4) and human Tip60 (Uniprot: Q92993) proteins with two major protein domains: N-terminal chromodomain and C-terminal catalytic MYST HAT domain. FIG. 1B: Tip60's histone acetylation mechanism. The chromodomain recognizes methylated lysine residues on histone proteins for Tip60 recuitment to target genes. The catalytic MYST HAT domain acetylates histone, which then relaxes the chromatin allowing for gene expression.

FIGS. 2A-2B: Target identification of acetyl-CoA binding pocket in Tip60's HAT domain. FIG. 2A: 3D Modeled structure of Tip60's catalytic HAT domain (PDB:2ou2) represented as cyan-colored ribbons with acetyl-CoA represented as orange licorice sticks. FIG. 2B: Acetyl-CoA binding pocket is present inside the Tip60's catalytic HAT domain represented in surface model.

FIGS. 3A-3C: CTB and CTPB as potential leads for Tip60 activation drug discovery. FIG. 3A: In silico GOLD protein-ligand docking software was used to dock CTB and CTPB ligands on Tip60's HAT domain (PDB:2ou2). Predicted chemical interactions, acceptors and donor as well as arene-H bonding are represented using Molecular Operating Environment (MOE). The ligand chemical structures are centered within interacting amino acids of Tip60's HAT domain that are either polar or greasy. FIG. 3B: CTB physically binds with Tip60 protein in vitro. Surface plasma resonance binding plot shows fraction of sites occupied (Y-axis) and compound concentration (M, X-axis) for CTB, CTPB and WM8014. K_{D} represents the estimated dissociation constant. FIG. 3C: CTB and CTPB partially prevent Tip60-knockdown associated locomotor defects in vivo. Dose-dependent response of CTB (left) and CTPB (right) drug administration from 10 nM to 100 μM or vehicle (DMSO). Statistical significance was calculated using one-way ANOVA with Dunnett's multiple comparisons. *p<0.05, **p<0.01, ****p<0.0001. Error bars indicate SEM.

FIGS. 4A-4D: Substructure-based virtual screening pipeline and in vivo testing for Tip60 HAT activators. FIG. 4A: CTB was used as a query ligand to screen compounds with a similar substructure in curated ZINC15 compound library. Top 100 compounds with similar structure as CTB were then individually docked on Tip60's HAT domain (PDB:2ou2) using GOLD protein-ligand docking software. Top 15 compounds with the best docking scores were selected and 10 out of these 15 compounds were synthesized for further testing, now referred to as C1-C10. FIGS. 4B-4D: In vivo testing of C1-C10 compounds using Drosophila Tip60 knockdown model in locomotor speed assay revealed high-performing compounds (FIG. 4B), low-performing compounds (FIG. 4C) and non-performing compound (FIG. 4D). Compounds were prepared using DMSO as vehicle and incorporated into the fly food. For control, equal amount of DMSO (vehicle) was mixed with the food. Statistical significance was calculated using one-way ANOVA with Dunnett's multiple comparisons. *p<0.05; **p<0.01, ***p<0.001, p<0.0001. Error bars indicate SEM.

FIGS. 5A-5B: Pharmacophore based virtual screening pipeline for Tip60 HAT activators. FIG. 5A: Synthesis of compounds. CTB was used as a query ligand to screen compounds with a similar pharmacophoric model in curated ZINC15 compound library. Top ˜19,000 compounds with similar pharmacophore as CTB were then individually docked on Tip60's HAT domain (PDB:2ou2) using GOLD protein-ligand docking software. Using a filter for Gold docking score >78, top 38 compounds with best docking scores were selected and 13 of these compounds were custom synthesized for further testing, now referred to as P1-P13. FIG. 5B: Pharmacophore modeling on CTB ligand. Using the Molecular Operating Environment (MOE), six pharmacophore features for CTB were screened as follows: F1 aromatic ring; F2 aromatic ring; F3 hydrogen donor; F4 hydrogen acceptor; F5 hydrophobic; and F6 hydrophobic. Different combinations of these pharmacophore features were used for virtual screening on ZINC15 compound library.

FIGS. 6A-6D: Novel Tip60 HAT inhibitors identified via in vitro histone acetylation assay. FIG. 6A: Histone acetylation assay shows Tip60 mediated Histone H4 acetylation in presence of vehicle (DMSO), WM8014 (known Tip60 inhibitor) or pharmacophore-based P4, P3, P2 and P1 compounds at 20 μM concentration. Background contains all reaction mixture, however, Histone H4 peptide is added after stopping the reaction to account for background fluorescence. Y-axis represents arbitrary fluorescence units (AFU) acquired on a spectrophotometer. Statistical significance was calculated using one-way ANOVA with Dunnett's multiple comparisons to Tip60+DMSO sample. **p<0.01; ***p<0.001; ****p<0.0001. Error bars indicate SEM. FIG. 6B: Calculation of specific enzyme activity (pmol/min/μg) and percent decrease in Tip60's HAT activity for different treatments. FIG. 6C: Compound structures where m (molecule) and number (docking score) is indicated. FIG. 6D: Histone acetylation assay using CTB and Calculation of specific enzyme activity (pmol/min/μg) and percent decrease in Tip60's HAT activity.

FIGS. 7A-7D: Novel Tip60 HAT activators identified via in vitro histone acetylation assay. FIGS. 7A-7B: Histone acetylation assay shows Tip60 (FIG. 7A) or p300 (FIG. 7B) mediated Histone H4 acetylation in presence of vehicle (DMSO), CTB (p300 HAT activator) or pharmacophore based P6, P9, P10 and P11, P13 compounds at 20 μM concentration. Background contains all reaction mixture, except for Histone H4 peptide which is added after stopping the reaction to account for background fluorescence. Y-axis represents arbitrary fluorescence units (AFU) acquired on a spectrophotometer. Statistical significance was calculated using one-way ANOVA with Dunnett's multiple comparisons to Tip60+DMSO sample. **p<0.01; ***p<0.001; ****p<0.0001. Error bars indicate SEM. FIG. 7C: Calculation of specific enzyme activity (pmol/min/μg) and percent increase in Tip60 or p300's HAT activity for different treatments. FIG. 7D: Compound structures where m (molecule) and number (docking score) is indicated.

FIGS. 8A-8B: P6, P10, P13 compounds rescue locomotor speed defect in Drosophila Tip60 knockdown model in vivo. High-performing compounds identified via in vivo screening in Tip60-RNAi mediated knockdown in the nervous system using elavC155 pan-neuronal Gal4 driver. FIG. 8A: Compound efficacy on ability of 3^{rd} instar larva to cross 0.4 cm grids lines in 30 seconds. FIG. 8B: Compound efficacy on ability of 3^{rd} instar larva to cross entire plate and reach the wall. Compounds were prepared using DMSO as vehicle and incorporated into the fly food and fed to the progeny larvae since early developmental stages. For control, equal amount of DMSO (vehicle) was mixed with the food. Statistical significance was calculated using one-way ANOVA with Dunnett's multiple comparisons. *p<0.05; **p<0.01, ***p<0.001, ****p<0.0001. Error bars indicate SEM. Minimum of 100 larvae used per experiment.

FIGS. 9A-9C: P10 and P13 compounds protect against functional neuronal deficits and lethality in a well characterized Drosophila Alzheimer's disease model. APP transgene was driven by elavC155 pan-neuronal Gal4 driver. FIGS. 9A-9B: Compound efficacy on ability of 3^{rd} instar APP larva to cross 0.4 cm grids lines in 30 seconds (FIG. 9A) and reach the boundary wall (FIG. 9B). FIG. 9C: Compound efficacy on the percent of adult APP flies that eclose over a 7-day period. Compounds were prepared using DMSO as vehicle and incorporated into the fly food and fed to the progeny larvae since early developmental stages. For control, equal amount of DMSO (vehicle) was mixed with the food. Statistical significance was calculated using one-way ANOVA with Dunnett's multiple comparisons. *p<0.05; **p<0.01, ***p<0.001, p<0.0001. Error bars indicate SEM. Minimum of 100 larvae used per experiment.

FIG. 10: Tip60 HAT activation as a unique therapeutic strategy for restoring histone acetylation homeostasis mediated cognition in Alzheimer's Disease (AD). In the healthy brain (left), the balance between Tip60 HAT and HDAC2 enzymes maintains histone acetylation homeostasis necessary for chromatin relaxation and neural gene expression that drives dynamic neural processes. However, in the AD brain (right), loss of Tip60 HAT and/or gain of HDAC2 activity results in reduced histone acetylation levels that condenses chromatin and represses critical neuronal genes resulting in cognitive decline. Tip60 activators selectively enhance its catalytic function to restore histone acetylation levels in the AD brain that should promote expression of critical neural genes and therefore, ameliorate cognitive function in the AD brain.

FIG. 11: non-limiting schematic representation of a histone acetylation transferase design.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science and organic chemistry are those well-known and commonly employed in the art. It should be understood that the order of steps or order for performing certain actions is immaterial, so long as the present teachings remain operable. Moreover, two or more steps or actions can be conducted simultaneously or not.

As used herein, the articles “a” and “an” refer to one or to more than one (i.e., to at least one) of the grammatical object of the article. By way of example, “an element” means one element or more than one element.

As used herein, the term “alkenyl,” employed alone or in combination with other terms, means, unless otherwise stated, a stable monounsaturated or diunsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined elsewhere herein, connected to the rest of the molecule via an oxygen atom, such as, for example, methoxy, ethoxy, 1-propoxy, 2-propoxy (or isopropoxy) and the higher homologs and isomers. A specific example is (C₁-C₃) alkoxy, such as, but not limited to, ethoxy and methoxy.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. A specific embodiment is (C₁-C₆) alkyl, such as, but not limited to, ethyl, methyl, isopropyl, isobutyl, n-pentyl, n-hexyl and cyclopropylmethyl.

As used herein, the term “alkynyl” employed alone or in combination with other terms means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C═CH. The term “homopropargylic” refers to a group exemplified by —CH₂CH₂—C═CH.

As used herein, the term “aromatic” refers to a carbocycle or heterocycle with one or more polyunsaturated rings and having aromatic character, i.e., having (4n+2) delocalized x (pi) electrons, where ‘n’ is an integer.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl and naphthyl. Aryl groups also include, for example, phenyl or naphthyl rings fused with one or more saturated or partially saturated carbon rings (e.g., bicyclo[4.2.0] octa-1,3,5-trienyl, or indanyl), which can be substituted at one or more carbon atoms of the aromatic and/or saturated or partially saturated rings.

As used herein, the term “aryl-(C₁-C₆)alkyl” refers to a functional group wherein a one to six carbon alkylene chain is attached to an aryl group, e.g., —CH₂CH₂-phenyl or —CH₂-phenyl (or benzyl). Specific examples are aryl —CH₂— and aryl-CH (CH₃)—. The term “substituted aryl-(C₁-C₆)alkyl” refers to an aryl-(C₁-C₆)alkyl functional group in which the aryl group is substituted. A specific example is substituted aryl(CH₂)—. Similarly, the term “heteroaryl-(C₁-C₆)alkyl” refers to a functional group wherein a one to three carbon alkylene chain is attached to a heteroaryl group, e.g., —CH₂CH₂-pyridyl. A specific example is heteroaryl-(CH₂)—. The term “substituted heteroaryl-(C₁-C₆)alkyl” refers to a heteroaryl-(C₁-C₆)alkyl functional group in which the heteroaryl group is substituted. A specific example is substituted heteroaryl-(CH₂)—.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆) cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3] undecanyl.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “halide” refers to a halogen atom bearing a negative charge. The halide anions are fluoride (F⁻), chloride (Cl⁻), bromide (Br⁻), and iodide (I⁻).

As used herein, the term “halo” or “halogen” alone or as part of another substituent refers to, unless otherwise stated, a fluorine, chlorine, bromine, or iodine atom.

As used herein, the term “heteroalkyl” by itself or in combination with another term refers to, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, and —CH₂CH₂S(═O)CH₃. Up to two heteroatoms may be consecutive, such as, for example, —CH₂NH—OCH₃, or —CH₂CH₂SSCH₃.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent refers to, unless otherwise stated, an unsubstituted or substituted, stable, mono- or multi-cyclic heterocyclic ring system that comprises carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

This listing of heterocyclyl and heteroaryl moieties is intended to be representative and not limiting.

As used herein, the term “pharmaceutical composition” or “composition” refers to a mixture of at least one compound useful within the disclosure with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the disclosure, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

The term “prevent,” “preventing,” or “prevention” as used herein means avoiding or delaying the onset of symptoms associated with a disease or condition in a subject that has not developed such symptoms at the time the administering of an agent or compound commences. Disease, condition and disorder are used interchangeably herein.

By the term “specifically bind” or “specifically binds” as used herein is meant that a first molecule preferentially binds to a second molecule (e.g., a particular receptor or enzyme), but does not necessarily bind only to that second molecule.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “substituted” refers to that an atom or group of atoms has replaced hydrogen as the substituent attached to another group.

As used herein, the term “substituted alkyl,” “substituted cycloalkyl,” “substituted alkenyl,” or “substituted alkynyl” refers to alkyl, cycloalkyl, alkenyl or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C═N, —C(═O)NH₂, —C(═O)NH(C₁-C₆)alkyl, —C(═O)N((C₁-C₆)alkyl)₂, —SO₂NH₂, —SO₂NH(C₁-C₆ alkyl), —SO₂N (C₁-C₆ alkyl)₂, —C(═NH) NH₂, and —NO₂, in certain embodiments containing one or two substituents independently selected from halogen, —OH, alkoxy, —NH₂, trifluoromethyl, —N(CH₃)₂, and —C(═O)OH, in certain embodiments independently selected from halogen, alkoxy and —OH. Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet another embodiments, the substituents vary in number between one and two. In yet other embodiments, the substituents are independently selected from the group consisting of C₁-C₆ alkyl, —OH, C₁-C₆ alkoxy, halo, amino, acetamido and nitro. As used herein, where a substituent is an alkyl or alkoxy group, the carbon chain may be branched, straight or cyclic.

In certain embodiments, each occurrence of alkyl or cycloalkyl is independently optionally substituted with at least one substituent selected from the group consisting of C₁-C₆ alkyl, halo, —OR, phenyl (thus yielding, in non-limiting examples, optionally substituted phenyl-(C₁-C₃ alkyl), such as, but not limited to, benzyl or substituted benzyl) and —N(R)(R), wherein each occurrence of R is independently H, C₁-C₆ alkyl or C₃-C₈ cycloalkyl. In other embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR, —N(R)(R), —NO₂, —S(═O)₂N (R)(R), acyl, and C₁-C₆ alkoxycarbonyl, wherein each occurrence of R is independently H, C₁-C₆ alkyl or C₃-C₈ cycloalkyl. In yet other embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halo, —CN, —OR, —N(R)(R), and C₁-C₆ alkoxycarbonyl, wherein each occurrence of R is independently H, C₁-C₆ alkyl or C₃-C₈ cycloalkyl.

Unless otherwise noted, when two substituents are taken together to form a ring having a specified number of ring atoms (e.g., R² and R³ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted with one or more substituents, non-limiting examples including a carbonyl (C═O).

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The terms “treat,” “treating,” and “treatment,” as used herein, means reducing the frequency or severity with which symptoms of a disease or condition are experienced by a subject by virtue of administering an agent or compound to the subject.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual and partial numbers within that range, for example, 1, 2, 2.7, 3, 4, 5, 5.3, and 6. This applies regardless of the breadth of the range.

### DESCRIPTION

### Two Distinct in Silico Ligand-Based Virtual Screening Approaches Successfully Identify Novel Tip60 HAT Activators

Identification and testing of a lead compound with beneficial activity that can be further modified for specificity towards a biological target serves as a crucial first step in drug discovery process. Although several naturally occurring and synthetic compounds, such as garcinol, curcumin and anacardic acid, are known to inhibit HAT activity, finding compounds that instead activate HAT activity remains challenging.

However, in search of potent p300 HAT inhibitors using anacardic acid as a synthon, CTPB was found, that enhanced p300 activity in an in vitro histone acetylation assay. CTPB was found to physically bind with p300 HAT in vitro using surface plasma resonance (SPR) and the intermolecular interactions significantly altered CTPB conformation when bound to the active site in p300 HAT. To enhance the HAT activation potency, modifications of CTPB resulted in discovery of CTB, an amide derivative of anacardic acid lacking the pentadecyl hydrocarbon chain of CTPB, that was found to be more potent HAT activator than CTPB in in vitro histone acetylation assay and exhibits higher binding affinity than CTPB in in silico molecular docking on p300 HAT. Further studies have since revealed that CTB and CTPB exert their HAT activation effect by inducing structural changes in the p300 HAT structure as observed using Raman spectroscopy.

Although CTB and CTPB are known to enhance p300 HAT activity, their ability to interact with Tip60 has not been previously tested which is a prerequisite for being accepted as lead compounds that can be further modified for Tip60-activator drug design. Using molecular docking, it was predicted herein that both CTB and CTPB interact favorably with Tip60's HAT domain, suggesting these compounds have the ability to interact with Tip60 enzyme as well. Further, using surface plasma resonance technique, it was confirmed herein that both CTB and CTPB were able to directly bind with Tip60 protein in vitro with CTB displaying a slightly higher binding affinity for Tip60 than CTPB. Additionally, the ability of CTB to directly enhance Tip60's HAT activity in in vitro histone acetylation assay resulting in higher acetylation even at low micromolar concentrations indicates that CTB-Tip60 physical binding exerts functional effects in Tip60's HAT domain. Lastly, as described herein, it was found that both, CTB and CTPB, can rescue locomotor deficits associated with Tip60 pan-neuronal knockdown in Drosophila larval brain in vivo with CTB showing more robust therapeutic effects than CTPB. Therefore, these in silico, in vitro and in vivo studies together provide a strong foundation for CTB as potent lead compound for Tip60-specific HAT activation drug design that can be further modified for increased specificity.

Virtual screening has become a standard tool in modern drug discovery that utilizes computational methods for screening of huge and diverse compound libraries to identify those structures which are most likely to bind to the desired target (Lill 2013; Rudrapal and Egbuna 2022). Broadly, virtual screening can be performed via ligand-based or structure-based methods that utilizes knowledge of a ligand known to bind to the desired target or the 3D structure of the target protein, respectively. Here, since CTB was identified as a suitable lead for Tip60-activator drug design, ligand-based virtual screening was performed utilizing two different approaches for identification of optimized lead compounds with increased Tip60 specificity. First, CTB substructure-based virtual screening was performed with the goal of finding compounds that retain CTB's core substructure with additional favorable functional groups that may improve the binding affinity towards Tip60. Since the relative position of trifluoromethyl (—CF₃) and chlorine (—Cl) substituents in CTB has been shown to be critical for HAT activation and these halogens were retained in this approach, the resulting modulators were predicted to retain overall HAT activation function.

However, due to high structural similarity with CTB that is known to also target p300 HAT enzyme, these modulators may not be uniquely specific towards Tip60. Indeed, it was found that the top 10 compounds (C1-C10) with highest docking scores on Tip60's HAT domain only slightly increased docking predictions from a score of 52 for CTB to 58-68 for these compounds, suggesting only a minimal improvement in Tip60-specific targeting. However, most of these compounds showed significant rescue of Tip60 knockdown mediated neuronal locomotor deficits with several compounds showing a significantly better response than CTB.

To overcome the low Tip60-specificity issue in the ligand-based virtual screening approach, a second approach was utilized that involves CTB pharmacophore-based virtual screening, with the goal of finding compounds that retain CTB's functional characteristics without necessarily retaining the core substructure. Since this approach broadened the compound search filters to allow for structural modifications without the restricted substructure with halogens, these modulators are expected to be uniquely specific towards Tip60 with the caviat that not all identified compounds would be HAT activators. Indeed, as described herein, it was found that the top 13 compounds (P1-P13) with highest docking scores on Tip60's HAT domain showed marked increase in docking predictions from a score of 52 for CTB to 78-81 for these P compounds, suggesting these compounds may have better specificity to Tip60. However, upon testing them in our in vitro histone acetylation assay, we found that these modulators contained a mixture of Tip60 HAT activators as well as inhibitors. After filtering out the inhibitors from this Tip60 modulator pool, several compounds were identified that show strong predicted binding affinity towards Tip60 enzyme in silico and have the ability to specifically activate Tip60's HAT function in vitro. Together, these results show that several compounds designed via substructure-based and pharmacophore-based virtual screening are potent Tip60 HAT activators with high Tip60-specificity and efficacy as observed in in vitro and in vivo assays.

### Tip60 HAT Activation as a Novel Therapeutic Strategy for Alzheimer's Disease (AD)

Small molecular compounds designed to increase histone acetylation are currently a research hotspot for development of cognitive enhancing drugs for several neurodegenerative diseases including AD. Accordingly, several HDAC inhibitors show promise in reversing cognition in AD mice models by increasing global histone acetylation. Indeed, two HDAC inhibitors, Vorinostat (SAHA, Zolinza) (NCT03056495) and Nicotinamide (Vitamin B3) (NCT03061474) have now advanced to Phase I and Phase II clinical trials for treatment of AD, respectively.

Unfortunately, most available HDAC inhibitors cause detrimental side effects due to their non-selective nature in targeting multiple HDACs resulting in non-specific global hyperacetylation and gene activation and inhibition of required HDAC function in cognition, thereby limiting their use in the development of AD therapeutics. For example, Vorinostat is a pan-inhibitor for class I and class II HDACs (HDACs 1-10) and although effective in AD animal models, is not considered beneficial at safe doses for humans and its negative side effects likely prohibit its use for AD population. Similarly, Nicotinamide is a pan-inhibitor for all class III sirtuin HDACs (SIRT1-7) that has failed to improve cognitive function in Phase II clinical trial for treatment of mild to moderate AD and shown to exacerbate Parkinson's pathology.

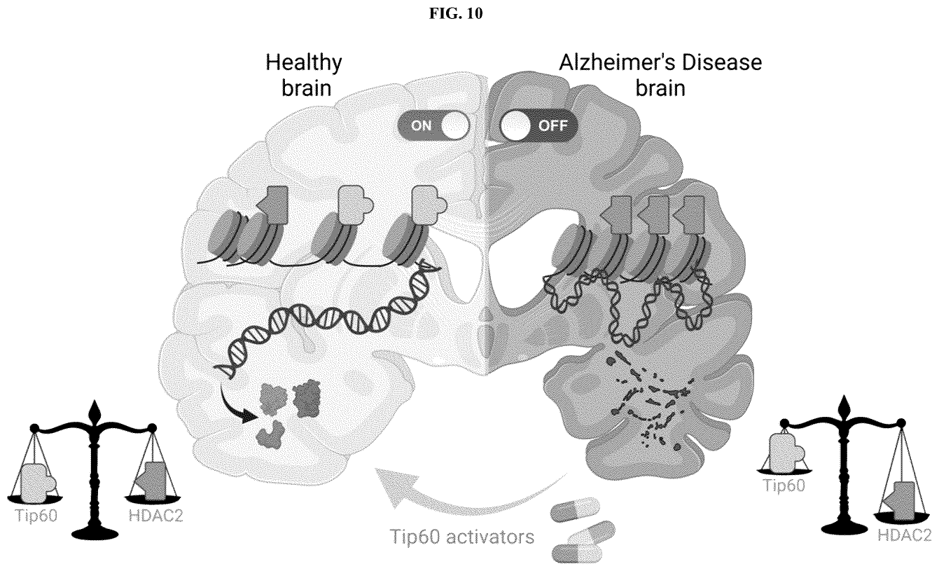

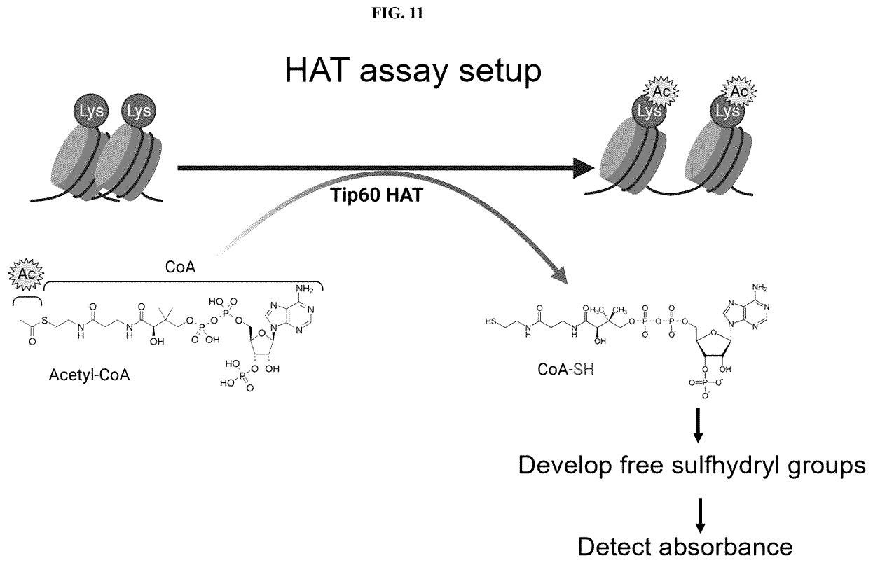

Alternatively, fine tuning of histone acetylation levels with activation of specific HATs is a promising new therapeutic strategy for treating neurodegenerative diseases. However, until now, HAT activation therapies have been limited by the difficulty of designing small molecule modulators that are able to activate, and not inhibit, enzyme activity. Thus far, the two commonly tested HAT activators include CTPB and SPV106 that are known to activate p300 and PCAF HAT activity, respectively, but their therapeutic use are limited by cell-impermeability and inhibition of p300/CBP activity along with pro-apoptotic function in dividing cells. Here, the barrier to effective HAT activation therapeutics has been overcome by designing a novel pool of small molecule modulators that show high specificity towards enhancing Tip60's HAT activity which is reduced in the AD brain. These results support a direct mode of action for these compounds that should provide a more selective way to induce Tip60 specific histone acetylation mediated cognitive enhancement benefits for treatment of AD (FIG. 10). In support of this concept, it is demonstrated herein that the pharmacophore based Tip60 activators P10 and P13 display consistent and significant rescue of both AD associated early-stage severe locomotion deficits and later stage lethality, supporting their therapeutic potential and effectiveness in the treatment of AD.

Intriguingly, a previous genome-wide chromatin binding and transcriptome studies revealed that APP AD larval brains exhibit robust enhanced HDAC2 and reduced Tip60 binding at predominantly identical genome-wide neuroplasticity genes with concomitant transcriptional dysregulation. Further, it has been shown herein that such Tip60/HDAC2 binding disruptions were conserved at orthologous human genes in AD patient hippocampi. These results support an exciting new model by which Tip60 and HDAC2 compete for binding at the same neuronal genes, with their access dependent upon the disease state. Notable, increasing Tip60 HAT activity in the APP AD brain protected against these alterations by significantly reducing both inappropriately enhanced HDAC2 levels and binding enrichment at Tip60 neuronal target genes, allowing for restoration of Tip60 mediated HA and gene expression. Thus, without wishing to be bound by any theory, it is proposed herein that pharmacological activation of the reduced levels of Tip60 found in the human and Drosophila AD brain recapitulates this mechanism to protect against early AD associated epigenetic neuronal gene repression caused by inappropriate HDAC2 enrichment and Tip60 reduction. In conclusion, the specific Tip60 HAT activator mediated restoration of neural histone acetylation patterns is a unique therapeutic strategy that provides a safer, more selective way for neuroepigenetic modulation in AD.

### General Discussion

The present disclosure provides, in one aspect, compounds that specifically promote Tip60 HAT activation as a therapeutic strategy for reversing cognition-associated histone acetylation marks that are altered early in AD progression to enhance Tip60's neuroprotective function. Therefore, the Tip60-specific epigenetic strategy allows for non-invasive and early Alzheimer's Disease therapeutics.

In certain embodiments, compounds of the disclosure show good Tip60-binding affinity predictions in silico, specific activation of Tip60's histone acetylation function in vitro, and ability to restore Tip60-associated functional neuronal deficits in vivo. In certain embodiments, compounds of the disclosure compounds directly target and activate the Tip60 HAT enzyme to restore the specific Tip60-mediated cognition-linked histone acetylation patterns that are disrupted during early AD stages for early and non-invasive therapeutic treatment. Further, Tip60 activators are also useful in treating, preventing, and/or ameliorating Huntington's Disease and Parkinson's Disease, for example.

Further, Tip60 is upregulated in several cancers, so Tip60 inhibitors find use in treating, ameliorating, and/or preventing such Tip60-dependent and/or Tip60-overexpressing cancers.

### Compounds

In one aspect, the disclosure provides a compound of Formula (I) or a pharmaceutically acceptable salt thereof:

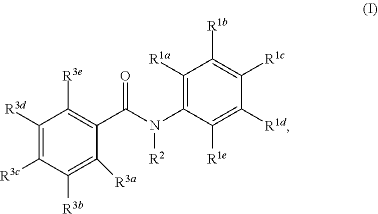

wherein:
R^{1a}, R^{1b}, and R^{1c} are each independently selected from the group consisting of H, optionally substituted C₁-C₆ alkyl, halogen, CN, and NO₂;
R^{1c} is halogen;
R^{1d} is C₁-C₃ haloalkyl;
R² is selected from the group consisting of H and optionally substituted C₁-C₆ alkyl;
R^{3a} and R^{3c} are each independently selected from the group consisting of optionally substituted C₁-C₁₂ alkoxy and optionally substituted C₁-C₂₀ alkyl; and
R^{3b}, R^{3c}, and R^{3d} are each independently selected from the group consisting of H, optionally substituted C₁-C₆ alkyl, halogen, CN, and NO₂.

In certain embodiments, R^{1a}, R^{1b}, and R^{1c} are each independently H. In certain embodiments, R^{1c} is Cl. In certain embodiments, R^{1d} is CF₃. In certain embodiments, R² is H.

In certain embodiments, R^{3a} is OEt. In certain embodiments, R^{3a} is OiPr. In certain embodiments, R^{3c} is

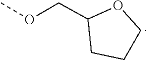

In certain embodiments, R^{3a} is

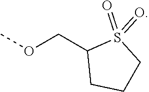

In certain embodiments, R^{3a} is

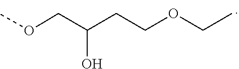

In certain embodiments, R^{3a} is

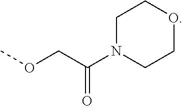

In certain embodiments, R^{3e} is H. In certain embodiments, R^{3e} is (CH₂)₁₄CH₃. In certain embodiments, R^{3e} is O(CH₂)₂N(CH₃)₂.

In certain embodiments, Formula (I) is selected from the group consisting of:

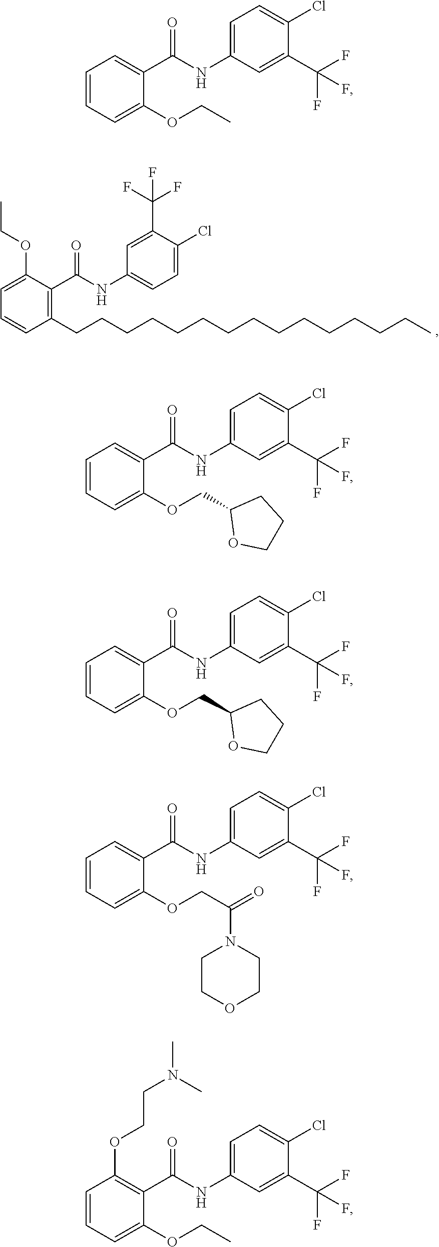

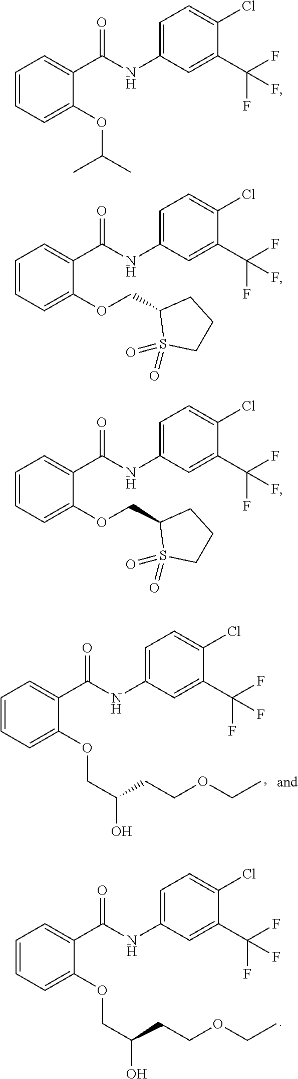

In one aspect, the disclosure provides a compound of Formula (II) or a pharmaceutically acceptable salt thereof:

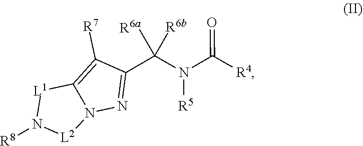

wherein:
R⁴ is selected from the group consisting of optionally substituted C₂-C₈ heteroaryl and C₆-C₁₀ aryl;
R⁵ is selected from the group consisting of H and optionally substituted C₁-C₆ alkyl; R^{6a} and R^{6b} are each independently selected from the group consisting of H and optionally substituted C₁-C₆ alkyl;
R⁷ is selected from the group consisting of H, optionally substituted C₁-C₆ alkyl, halogen, CN, and NO₂;
R⁸ is selected from the group consisting of H, optionally substituted C₁-C₆ alkyl, optionally substituted C₃-C₈ cycloalkyl, optionally substituted C₂-C₈ heterocycloalkyl, and C(═O)N(R^{A})(R^{B});
L¹ is —[C(R^{9a})(R^{9b})]₁₋₂—;
L² is —[C(R^{10a})(R^{10b})]₁₋₂—;
each occurrence of R^{9a} and R^{9b} is independently selected from the group consisting of H and optionally substituted C₁-C₆ alkyl;
each occurrence of R^{10a} and R^{10b} is independently selected from the group consisting of H and optionally substituted C₁-C₆ alkyl; and
R^{A} and R^{B} are each independently selected from the group consisting of H and optionally substituted C₁-C₆ alkyl, or R^{A} and R^{B} combine with the N atom to which they are bound to form an optionally substituted C₂-C₈ heterocycloalkyl.

In certain embodiments, R⁴ is selected from the group consisting of

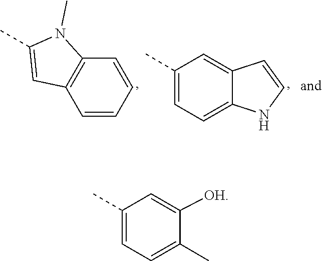

In certain embodiments, R⁵ is H. In certain embodiments, R^{6a} and R^{6b} are each independently H. In certain embodiments, R⁷ is H. In certain embodiments, R⁸ is cyclohexyl. In certain embodiments, R⁸ is C(═O)N(CH₃)₂. In certain embodiments, L¹ is —CH₂—. In certain embodiments, L² is —(CH₂)₂—. In certain embodiments, L² is —(CH₂)₃—.

In certain embodiments, the compound of Formula (II) is selected from the group consisting of:

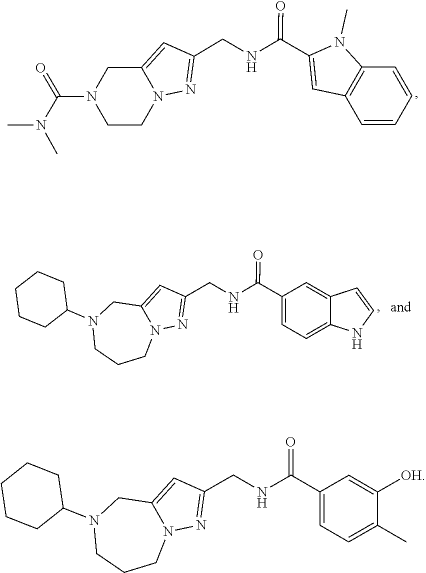 [SMILES: CN(C)C(=O)N1CCn2nc(CNC(=O)c3cc4ccccc4n3C)cc2C1 ; O=C(NCc1cc2n(n1)CCCN(C1CCCCC1)C2)c1ccc2[nH]ccc2c1 ; Cc1ccc(C(=O)NCc2cc3n(n2)CCCN(C2CCCCC2)C3)cc1O]

In certain embodiments, the disclosure provides a compound selected from the group consisting of:

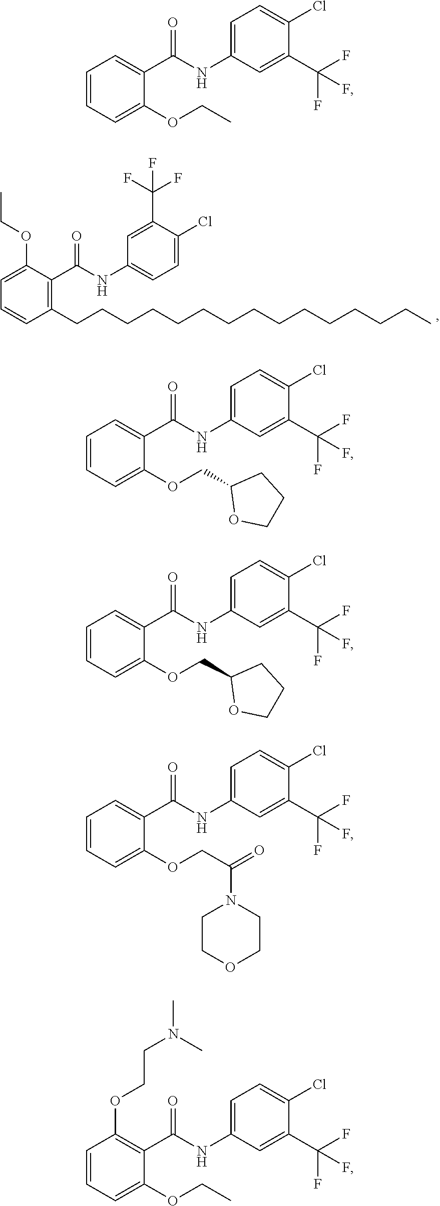

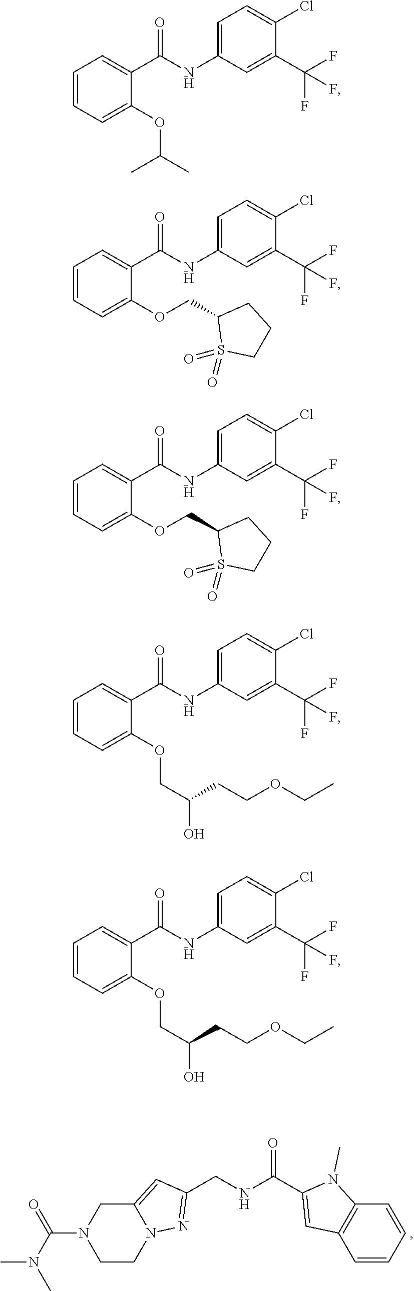

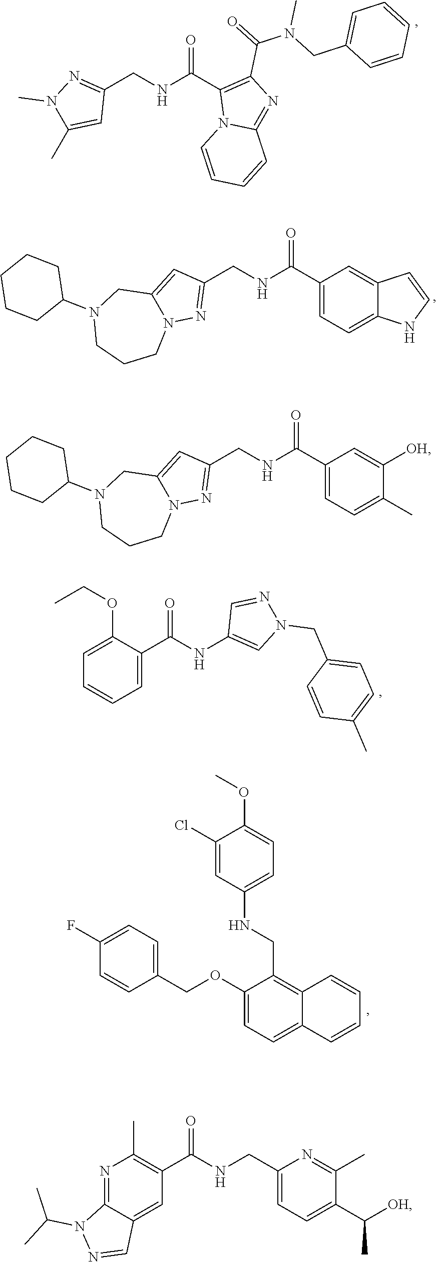 [SMILES: Cc1cc(CNC(=O)c2c(C(=O)N(C)Cc3ccccc3)nc3ccccn23)nn1C ; O=C(NCc1cc2n(n1)CCCN(C1CCCCC1)C2)c1ccc2[nH]ccc2c1 ; Cc1ccc(C(=O)NCc2cc3n(n2)CCCN(C2CCCCC2)C3)cc1O ; CCOc1ccccc1C(=O)Nc1cnn(Cc2ccc(C)cc2)c1 ; COc1ccc(NCc2c(OCc3ccc(F)cc3)ccc3ccccc23)cc1Cl ; Cc1nc2c(cnn2C(C)C)cc1C(=O)NCc1ccc([C@H](C)O)c(C)n1]

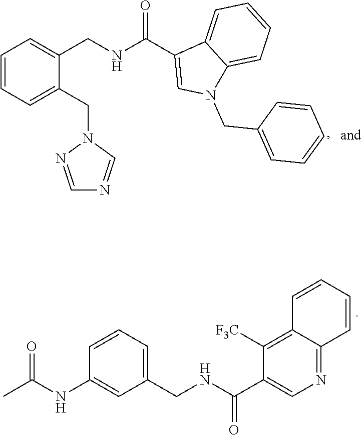 [SMILES: O=C(NCc1ccccc1Cn1cncn1)c1cn(Cc2ccccc2)c2ccccc12 ; CC(=O)Nc1cccc(CNC(=O)c2cnc3ccccc3c2C(F)(F)F)c1]

TABLE 1
Exemplary compounds of the disclosure

| Cmpd | Structure | Name |
|---|---|---|
| CTB (C1) | 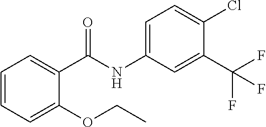 [SMILES: CCOc1ccccc1C(=O)Nc1ccc(Cl)c(C(F)(F)F)c1] | N-(4-chloro-3- (trifluoromethyl)phenyl)-2- ethoxybenzamide |
| CTBP | 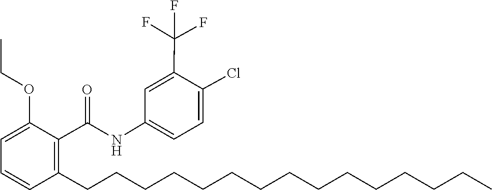 [SMILES: CCCCCCCCCCCCCCCc1cccc(OCC)c1C(=O)Nc1ccc(Cl)c(C(F)(F)F)c1] | N-(4-chloro-3- (trifluoromethyl)phenyl)-2-ethoxy- 6-pentadecylbenzamide |
| WM8014 | 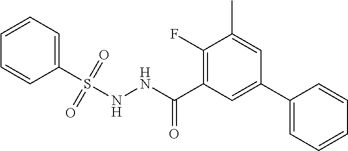 [SMILES: Cc1cc(-c2ccccc2)cc(C(=O)NNS(=O)(=O)c2ccccc2)c1F] | N′-(4-fluoro-5-methyl-[1,1′- bipheny1]-3- carbonyl)benzenesulfonohydrazide |
| P1 | 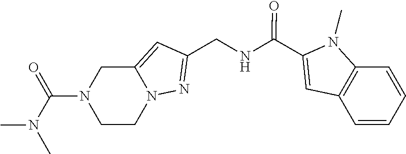 [SMILES: CN(C)C(=O)N1CCn2nc(CNC(=O)c3cc4ccccc4n3C)cc2C1] | N,N-dimethyl-2-((1-methyl-1H- indole-2-carboxamido)methyl)- 6,7-dihydropyrazolo[1,5- alpyrazine-5(4H)-carboxamide |
| P2 | 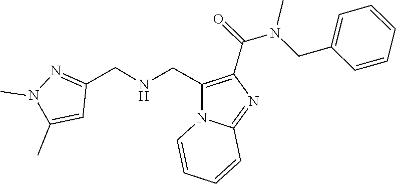 [SMILES: Cc1cc(CNCc2c(C(=O)N(C)Cc3ccccc3)nc3ccccn23)nn1C] | N-benzyl-3-((((1,5-dimethyl-1H- pyrazol-3- yl)methyl)amino)methyl)-N- methylimidazo[1,2-a]pyridine-2- carboxamide |
| P3 | 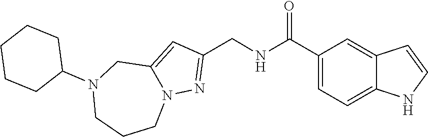 [SMILES: O=C(NCc1cc2n(n1)CCCN(C1CCCCC1)C2)c1ccc2[nH]ccc2c1] | N-((5-cyclohexyl-5,6,7,8- tetrahydro-4H-pyrazolo[1,5- a][1,4]diazepin-2-yl)methyl)-1H- indole-5-carboxamide |
| P4 | 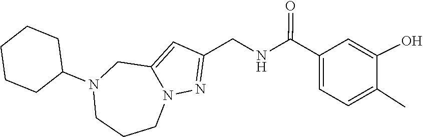 [SMILES: Cc1ccc(C(=O)NCc2cc3n(n2)CCCN(C2CCCCC2)C3)cc1O] | N-((5-cyclohexyl-5,6,7,8- tetrahydro-4H-pyrazolo[1,5- a][1,4]diazepin-2-yl)methyl)-3- hydroxy-4-methylbenzamide |
| P6 | 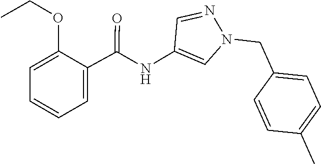 [SMILES: CCOc1ccccc1C(=O)Nc1cnn(Cc2ccc(C)cc2)c1] | 2-ethoxy-N-(1-(4-methylbenzyl)- 1H-pyrazol-4-yl)benzamide |
| P9 | 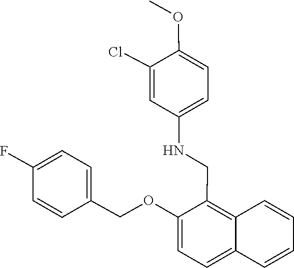 [SMILES: COc1ccc(NCc2c(OCc3ccc(F)cc3)ccc3ccccc23)cc1Cl] | 3-chloro-N-((2-((4- fluorobenzyl)oxy)naphthalen-1- yl)methyl)-4-methoxyaniline |
| P10 | 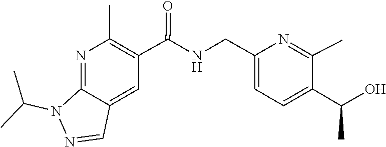 [SMILES: Cc1nc2c(cnn2C(C)C)cc1C(=O)NCc1ccc([C@H](C)O)c(C)n1] | (S)-N-((5-(1-hydroxyethyl)-6- methylpyridin-2-yl)methyl)-1- isopropyl-6-methyl-1H- pyrazolo[3,4-b]pyridine-5- carboxamide |
| P11 | 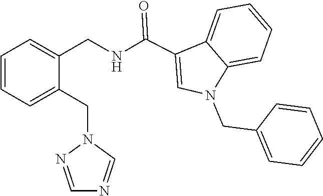 [SMILES: O=C(NCc1ccccc1Cn1cncn1)c1cn(Cc2ccccc2)c2ccccc12] | N-(2-((1H-1,2,4-triazol-1- yl)methyl)benzyl)-1-benzyl-1H- indole-3-carboxamide |
| P13 | 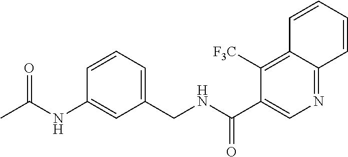 [SMILES: CC(=O)Nc1cccc(CNC(=O)c2cnc3ccccc3c2C(F)(F)F)c1] | N-(3-acetamidobenzyl)-4- (trifluoromethyl)quinoline-3- carboxamide |
| C2 | 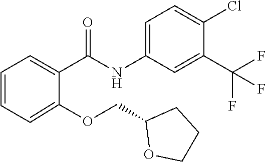 [SMILES: O=C(Nc1ccc(Cl)c(C(F)(F)F)c1)c1ccccc1OC[C@@H]1CCCO1] | (S)-N-(4-chloro-3- (trifluoromethyl)phenyl)-2- ((tetrahydrofuran-2- yl)methoxy)benzamide |
| C3 | 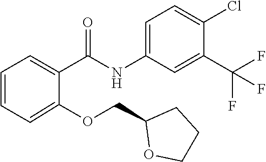 [SMILES: O=C(Nc1ccc(Cl)c(C(F)(F)F)c1)c1ccccc1OC[C@H]1CCCO1] | (R)-N-(4-chloro-3- (trifluoromethyl)phenyl)-2- ((tetrahydrofuran-2- yl)methoxy)benzamide |
| C4 | 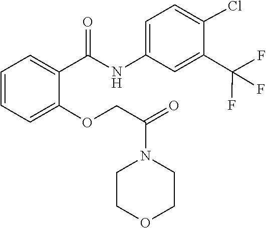 [SMILES: O=C(Nc1ccc(Cl)c(C(F)(F)F)c1)c1ccccc1OCC(=O)N1CCOCC1] | N-(4-chloro-3- (trifluoromethyl)phenyl)-2-(2- morpholino-2- oxoethoxy)benzamide |
| C5 | 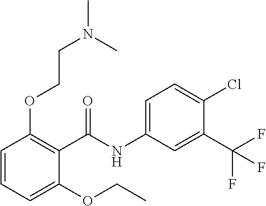 [SMILES: CCOc1cccc(OCCN(C)C)c1C(=O)Nc1ccc(Cl)c(C(F)(F)F)c1] | N-(4-chloro-3- (trifluoromethyl)phenyl)-2-(2- (dimethylamino)ethoxy)-6- ethoxybenzamide |
| C6 | 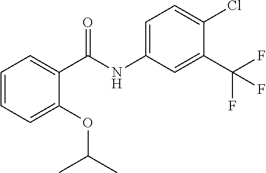 [SMILES: CC(C)Oc1ccccc1C(=O)Nc1ccc(Cl)c(C(F)(F)F)c1] | N-(4-chloro-3- (trifluoromethyl)phenyl)-2- isopropoxybenzamide |
| C7 | 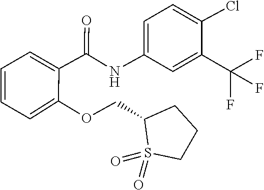 [SMILES: O=C(Nc1ccc(Cl)c(C(F)(F)F)c1)c1ccccc1OC[C@@H]1CCCS1(=O)=O] | (S)-N-(4-chloro-3- (trifluoromethyl)phenyl)-2-((1,1- dioxidotetrahydrothiophen-2- yl)methoxy)benzamide |
| C8 | 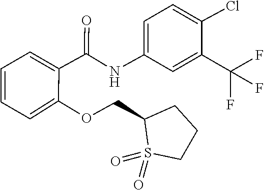 [SMILES: O=C(Nc1ccc(Cl)c(C(F)(F)F)c1)c1ccccc1OC[C@H]1CCCS1(=O)=O] | (R)-N-(4-chloro-3- (trifluoromethyl)phenyl)-2-((1,1- dioxidotetrahydrothiophen-2- yl)methoxy)benzamide |
| C9 | 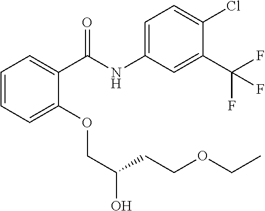 [SMILES: CCOCC[C@H](O)COc1ccccc1C(=O)Nc1ccc(Cl)c(C(F)(F)F)c1] | (S)-N-(4-chloro-3- (trifluoromethyl)phenyl)-2-(4- ethoxy-2- hydroxybutoxy)benzamide |
| C10 | 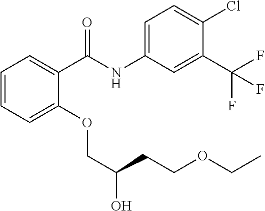 [SMILES: CCOCC[C@@H](O)COc1ccccc1C(=O)Nc1ccc(Cl)c(C(F)(F)F)c1] | (R)-N-(4-chloro-3- (trifluoromethyl)phenyl)-2-(4- ethoxy-2- hydroxybutoxy)benzamide |

The compounds of the disclosure may possess one or more stereocenters, and each stereocenter may exist independently in either the (R) or(S) configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or mixtures thereof, or in the case where two or more chiral center are present, all diastereomers or mixtures thereof.

In certain embodiments, the compounds of the disclosure exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed disclosure. The compounds of the disclosure may contain any of the substituents, or combinations of substituents, provided herein.

### Salts

The compounds described herein may form salts with acids or bases, and such salts are included in the present disclosure. The term “salts” embraces addition salts of free acids or bases that are useful within the methods of the disclosure. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present disclosure, such as for example utility in process of synthesis, purification or formulation of compounds useful within the methods of the disclosure.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the disclosure.

Suitable pharmaceutically acceptable base addition salts of compounds of the disclosure include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Methods

In one aspect, the disclosure provides a method of treating, preventing, and/or ameliorating a neurodegenerative disease. In certain embodiments, the method comprises administering to a subject in need thereof a therapeutically effective amount of at least one Tip60 activator or a pharmaceutically acceptable salt thereof.

In certain embodiments, the neurodegenerative disease is at least one selected from the group consisting of Alzheimer's Disease, Parkinson's Disease, Huntington's Disease, Amyotrophic Lateral Sclerosis (ALS), Frontotemporal Dementia, Multiple System Atrophy, Progressive Supranuclear Palsy, Corticobasal Degeneration, Lewy Body Dementia, Friedreich's Ataxia, Spinocerebellar Ataxia, Spinal Muscular Atrophy, Prion Diseases (such as Creutzfeldt-Jakob Disease), Wernicke-Korsakoff Syndrome, Kennedy's Disease (Spinal Bulbar Muscular Atrophy), Wilson's Disease, Niemann-Pick Disease, and Gerstmann-Sträussler-Scheinker Syndrome (GSS).

In certain embodiments, the subject is further administered at least one additional agent suitable for the treatment, prevention, and/or amelioration of a neurodegenerative disease.

In certain embodiments, the Tip60 activator is a compound of Formula (I) or a pharmaceutically acceptable salt thereof:

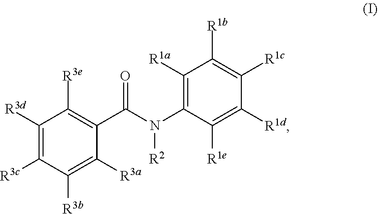

wherein:
R^{1a}, R^{1b}, and R^{1e} are each independently selected from the group consisting of H, optionally substituted C₁-C₆ alkyl, halogen, CN, and NO₂;
R^{1c} is halogen;
R^{1d} is C₁-C₃ haloalkyl;
R² is selected from the group consisting of H and optionally substituted C₁-C₆ alkyl;
R^{3c} and R^{3e} are each independently selected from the group consisting of optionally substituted C₁-C₁₂ alkoxy and optionally substituted C₁-C₂₀ alkyl; and
R^{3b}, R^{3c}, and R^{3d} are each independently selected from the group consisting of H, optionally substituted C₁-C₆ alkyl, halogen, CN, and NO₂.

In certain embodiments, R^{1a}, R^{1b}, and R^{1e} are each independently H. In certain embodiments, R^{1c} is Cl. In certain embodiments, R^{1d} is CF₃. In certain embodiments, R² is H.

In certain embodiments, R^{3a} is OEt. In certain embodiments, R^{3a} is OiPr. In certain embodiments, R^{3a} is

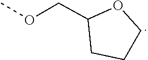

In certain embodiments, R^{3a} is

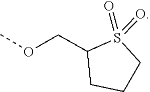

In certain embodiments, R^{3a} is

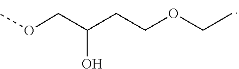

In certain embodiments, R^{3a} is

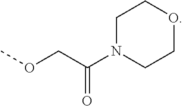

In certain embodiments, R^{3e} is H. In certain embodiments, R^{3e} is (CH₂)₁₄CH₃. In certain embodiments, R^{3e} is O(CH₂)₂N(CH₃)₂.

In certain embodiments, Formula (I) is selected from the group consisting of:

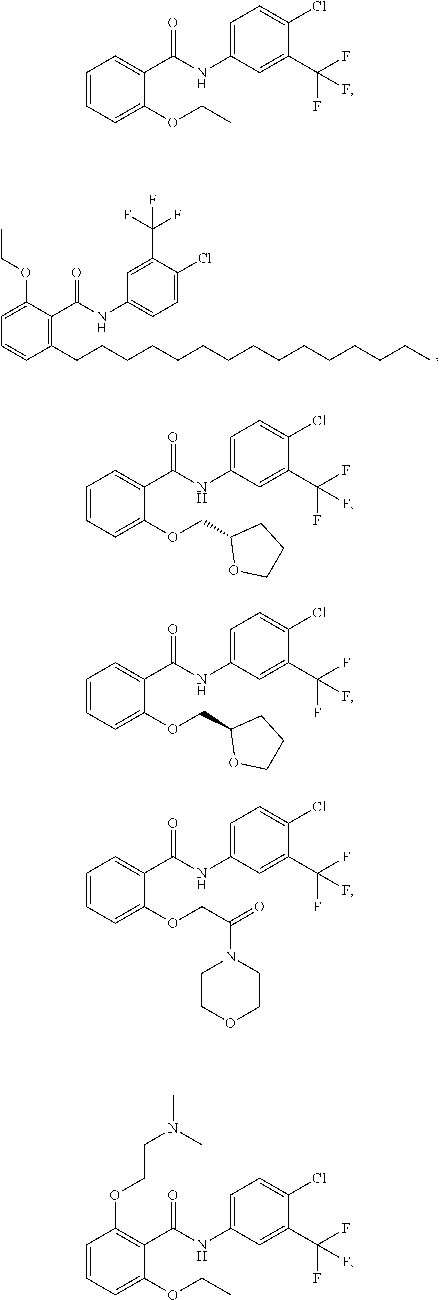

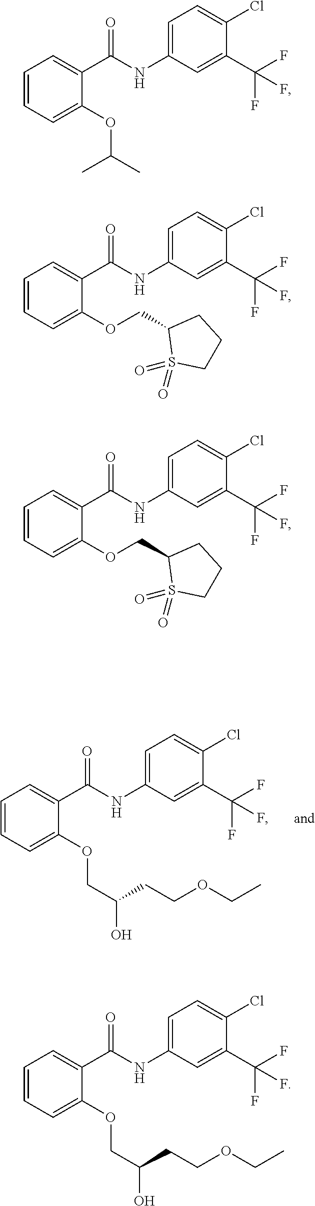 [SMILES: CC(C)Oc1ccccc1C(=O)Nc1ccc(Cl)c(C(F)(F)F)c1 ; O=C(Nc1ccc(Cl)c(C(F)(F)F)c1)c1ccccc1OC[C@@H]1CCCS1(=O)=O ; O=C(Nc1ccc(Cl)c(C(F)(F)F)c1)c1ccccc1OC[C@H]1CCCS1(=O)=O ; CCOCC[C@H](O)COc1ccccc1C(=O)Nc1ccc(Cl)c(C(F)(F)F)c1 ; "and" ; CCOCC[C@@H](O)COc1ccccc1C(=O)Nc1ccc(Cl)c(C(F)(F)F)c1]

In certain embodiments, the Tip60 activator is a compound of Formula (II) or a pharmaceutically acceptable salt thereof:

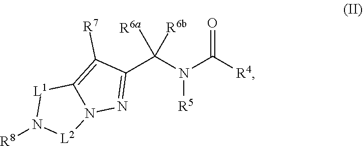

wherein:
R⁴ is selected from the group consisting of optionally substituted C₂-C₈ heteroaryl and C₆-C₁₀ aryl;
R⁵ is selected from the group consisting of H and optionally substituted C₁-C₆ alkyl;
R^{6a} and R^{6b} are each independently selected from the group consisting of H and optionally substituted C₁-C₆ alkyl;
R⁷ is selected from the group consisting of H, optionally substituted C₁-C₆ alkyl, halogen, CN, and NO₂;
R⁸ is selected from the group consisting of H, optionally substituted C₁-C₆ alkyl, optionally substituted C₃-C₈ cycloalkyl, optionally substituted C₂-C₈ heterocycloalkyl, and C(═O)N(R^{A})(R^{B});
L¹ is —[C(R^{9a})(R^{9b})]₁₋₂—;
L² is —[C(R^{10a})(R^{10b})]₁₋₂—;
each occurrence of R^{9a} and R^{9b} is independently selected from the group consisting of H and optionally substituted C₁-C₆ alkyl;
each occurrence of R^{10a} and R^{10b} is independently selected from the group consisting of H and optionally substituted C₁-C₆ alkyl; and
R^{A} and R^{B} are each independently selected from the group consisting of H and optionally substituted C₁-C₆ alkyl, or R^{A} and R^{B} combine with the N atom to which they are bound to form an optionally substituted C₂-C₈ heterocycloalkyl.

In certain embodiments, R⁴ is selected from the group consisting of

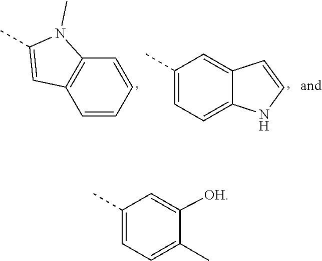

In certain embodiments, R⁵ is H. In certain embodiments, R^{6a} and R^{6b} are each independently H. In certain embodiments, R⁷ is H. In certain embodiments, R⁸ is cyclohexyl. In certain embodiments, R⁸ is C(═O)N(CH₃)₂. In certain embodiments, L¹ is —CH₂—. In certain embodiments, L² is —(CH₂)₂—. In certain embodiments, L² is —(CH₂)₃—.

In certain embodiments, the compound of Formula (II) is selected from the group consisting of:

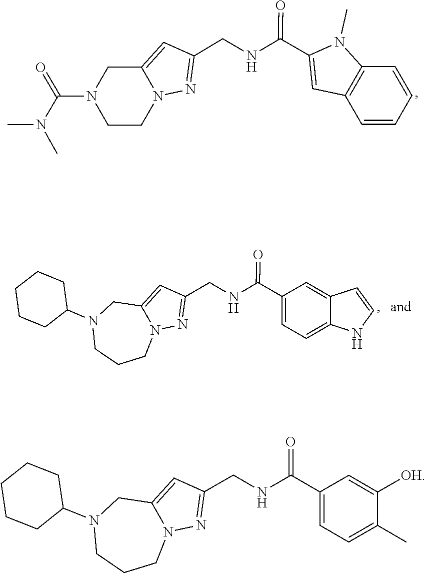 [SMILES: CN(C)C(=O)N1CCn2nc(CNC(=O)c3cc4ccccc4n3C)cc2C1 ; O=C(NCc1cc2n(n1)CCCN(C1CCCCC1)C2)c1ccc2[nH]ccc2c1 ; Cc1ccc(C(=O)NCc2cc3n(n2)CCCN(C2CCCCC2)C3)cc1O]

In certain embodiments, the Tip60 activator is selected from the group consisting of:

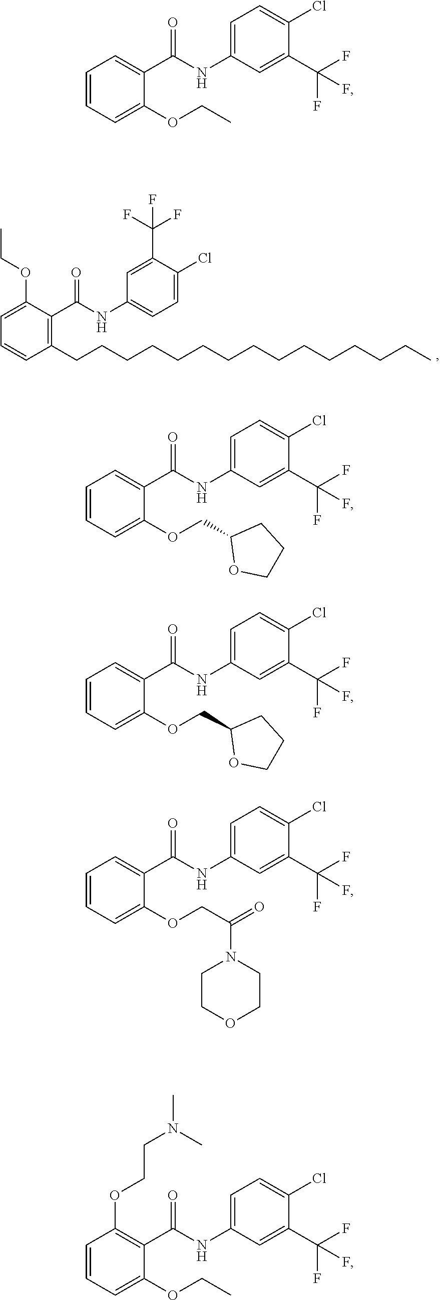

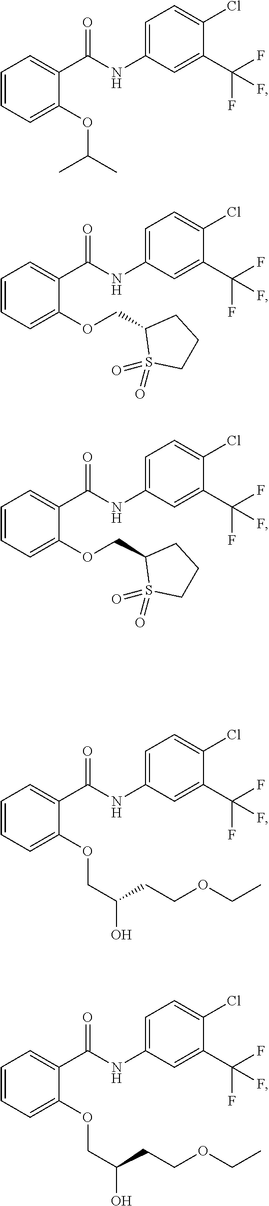 [SMILES: CC(C)Oc1ccccc1C(=O)Nc1ccc(Cl)c(C(F)(F)F)c1 ; O=C(Nc1ccc(Cl)c(C(F)(F)F)c1)c1ccccc1OC[C@@H]1CCCS1(=O)=O ; O=C(Nc1ccc(Cl)c(C(F)(F)F)c1)c1ccccc1OC[C@H]1CCCS1(=O)=O ; CCOCC[C@H](O)COc1ccccc1C(=O)Nc1ccc(Cl)c(C(F)(F)F)c1 ; CCOCC[C@@H](O)COc1ccccc1C(=O)Nc1ccc(Cl)c(C(F)(F)F)c1]

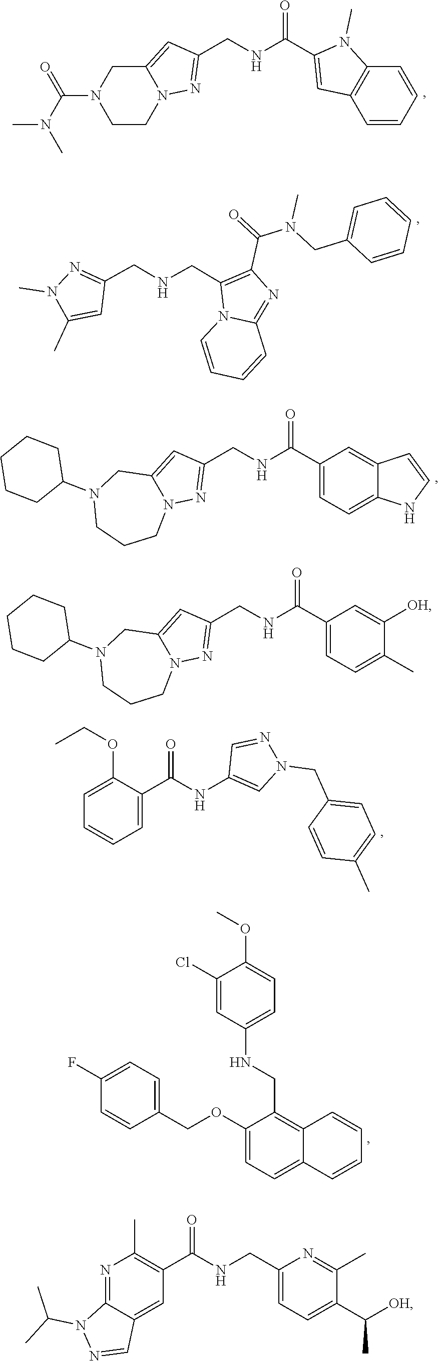

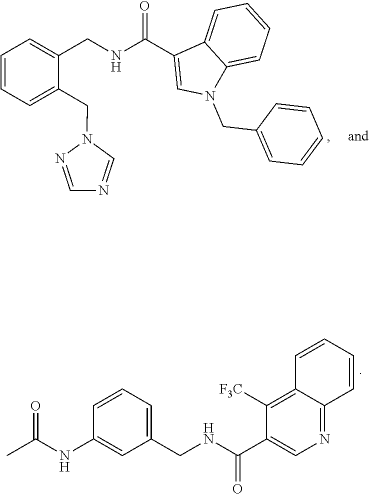 [SMILES: O=C(NCc1ccccc1Cn1cncn1)c1cn(Cc2ccccc2)c2ccccc12 ; CC(=O)Nc1cccc(CNC(=O)c2cnc3ccccc3c2C(F)(F)F)c1]

In another aspect, the disclosure provides a method of treating, preventing, and/or ameliorating a Tip60-dependent and/or Tip60-overexpressing cancer. In certain embodiments, the method comprises administering to a subject in need thereof a therapeutically effective amount of a Tip60 inhibitor or a pharmaceutically acceptable salt thereof.

In certain embodiments, the Tip60 activator is a compound of Formula (I) or a pharmaceutically acceptable salt thereof:

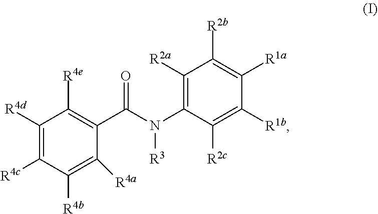

wherein:
R^{1a}, R^{1b}, and R^{1e} are each independently selected from the group consisting of H, optionally substituted C₁-C₆ alkyl, halogen, CN, and NO₂;
R^{1c} is halogen;
R^{1d} is C₁-C₃ haloalkyl;
R² is selected from the group consisting of H and optionally substituted C₁-C₆ alkyl;
R^{3a} and R^{3e} are each independently selected from the group consisting of optionally substituted C₁-C₁₂ alkoxy and optionally substituted C₁-C₂₀ alkyl; and
R^{3b}, R^{3c}, and R^{3d} are each independently selected from the group consisting of H, optionally substituted C₁-C₆ alkyl, halogen, CN, and NO₂.

In certain embodiments, R^{1a}, R^{1b}, and R^{1e} are each independently H. In certain embodiments, R^{1c} is Cl. In certain embodiments, R^{1d} is CF₃. In certain embodiments, R² is H.

In certain embodiments, R^{3a} is OEt. In certain embodiments, R^{3a} is OiPr. In certain embodiments, R^{3a} is

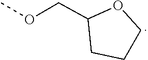

In certain embodiments, R^{3a} is

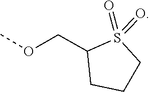

In certain embodiments, R^{3a} is

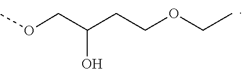

In certain embodiments, R^{3a} is

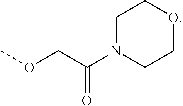

In certain embodiments, R^{3e} is H. In certain embodiments, R^{3e} is (CH₂)₁₄CH₃. In certain embodiments, R^{3e} is O(CH₂)₂N(CH₃)₂.

In certain embodiments, Formula (I) is selected from the group consisting of:

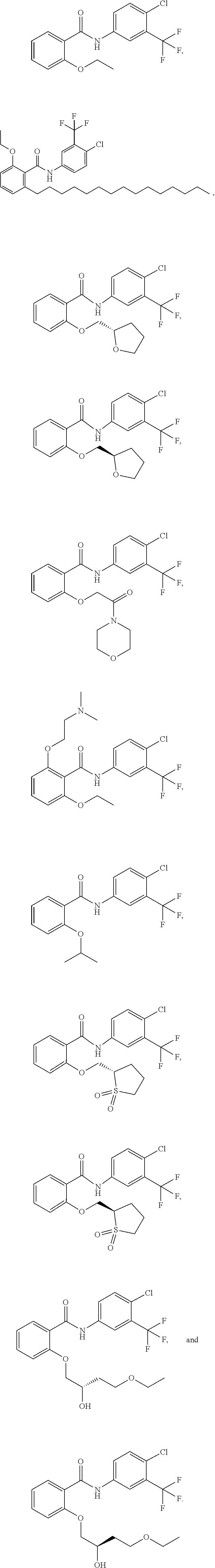

In certain embodiments, the Tip60 activator is a compound of Formula (II) or a pharmaceutically acceptable salt thereof:

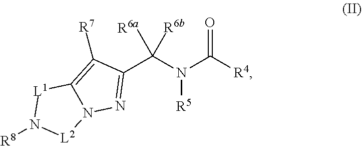

wherein:
R⁴ is selected from the group consisting of optionally substituted C₂-C₈ heteroaryl and C₆-C₁₀ aryl;
R⁵ is selected from the group consisting of H and optionally substituted C₁-C₆ alkyl;
R^{6a} and R^{6b} are each independently selected from the group consisting of H and optionally substituted C₁-C₆ alkyl;
R⁷ is selected from the group consisting of H, optionally substituted C₁-C₆ alkyl, halogen, CN, and NO₂;
R⁸ is selected from the group consisting of H, optionally substituted C₁-C₆ alkyl, optionally substituted C₃-C₈ cycloalkyl, optionally substituted C₂-C₈ heterocycloalkyl, and C(═O)N(R^{A})(R^{B});
L¹ is —[C(R^{9a})(R^{9b})]₁₋₂—;
L² is —[C(R^{10a})(R^{10b})]₁₋₂—;
each occurrence of R^{9c} and Rob is independently selected from the group consisting of H and optionally substituted C₁-C₆ alkyl;
each occurrence of R^{10a} and R^{10b} is independently selected from the group consisting of H and optionally substituted C₁-C₆ alkyl; and
R^{A} and R^{B} are each independently selected from the group consisting of H and optionally substituted C₁-C₆ alkyl, or R^{A} and R^{B} combine with the N atom to which they are bound to form an optionally substituted C₂-C₈ heterocycloalkyl.

In certain embodiments, R⁴ is selected from the group consisting of

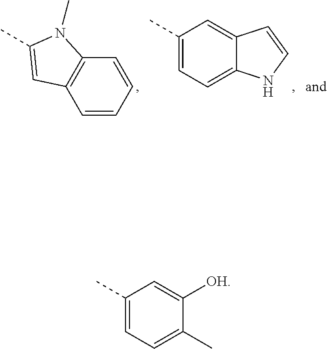

In certain embodiments, R⁵ is H. In certain embodiments, R^{6a} and R^{6b} are each independently H. In certain embodiments, R⁷ is H. In certain embodiments, R⁸ is cyclohexyl. In certain embodiments, R⁸ is C(═O)N(CH₃)₂. In certain embodiments, L¹ is —CH₂—. In certain embodiments, L² is —(CH₂)₂—. In certain embodiments, L² is —(CH₂)₃—.

In certain embodiments, the compound of Formula (II) is selected from the group consisting of:

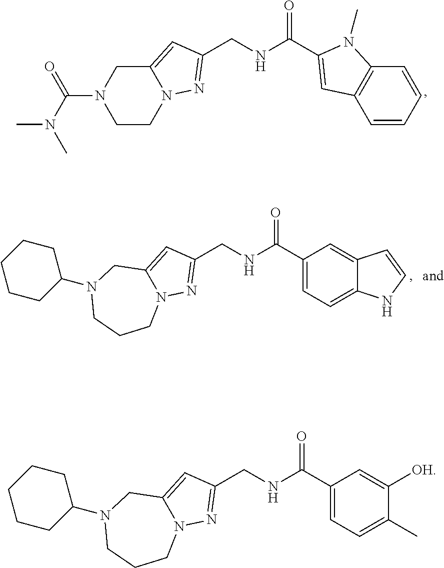 [SMILES: CN(C)C(=O)N1CCn2nc(CNC(=O)c3cc4ccccc4n3C)cc2C1 ; "," ; O=C(NCc1cc2n(n1)CCCN(C1CCCCC1)C2)c1ccc2[nH]ccc2c1 ; "," ; "and" ; Cc1ccc(C(=O)NCc2cc3n(n2)CCCN(C2CCCCC2)C3)cc1O]

In certain embodiments, the Tip60 activator is selected from the group consisting of:

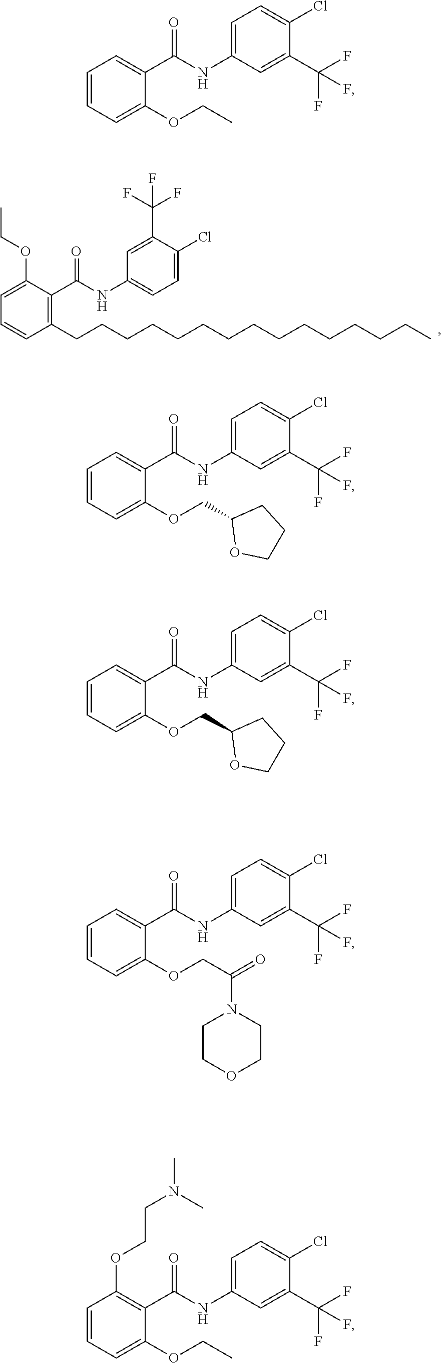 [SMILES: CCOc1ccccc1C(=O)Nc1ccc(Cl)c(C(F)(F)F)c1 ; CCCCCCCCCCCCCCCc1cccc(OCC)c1C(=O)Nc1ccc(Cl)c(C(F)(F)F)c1 ; O=C(Nc1ccc(Cl)c(C(F)(F)F)c1)c1ccccc1OC[C@@H]1CCCO1 ; O=C(Nc1ccc(Cl)c(C(F)(F)F)c1)c1ccccc1OC[C@H]1CCCO1 ; O=C(Nc1ccc(Cl)c(C(F)(F)F)c1)c1ccccc1OCC(=O)N1CCOCC1 ; CCOc1cccc(OCCN(C)C)c1C(=O)Nc1ccc(Cl)c(C(F)(F)F)c1]

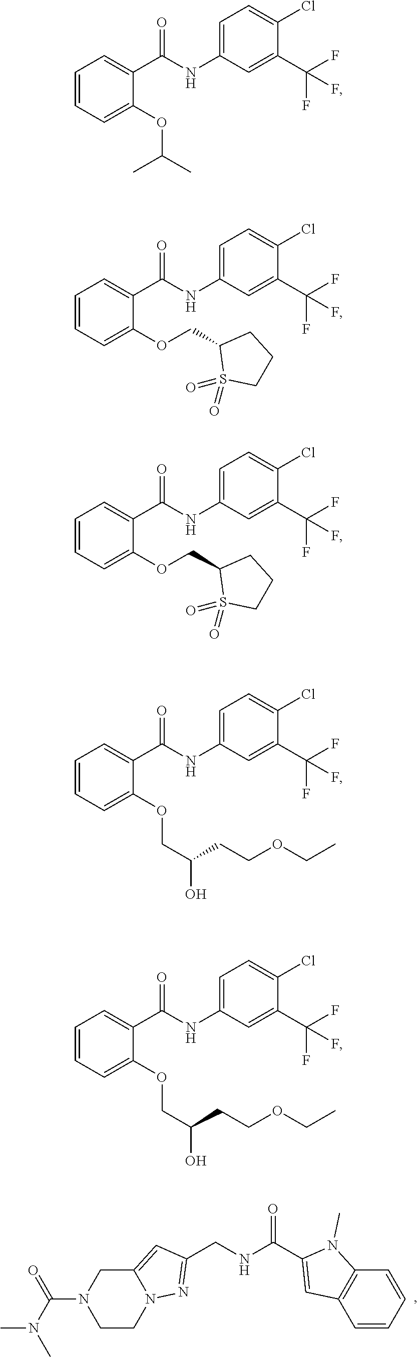

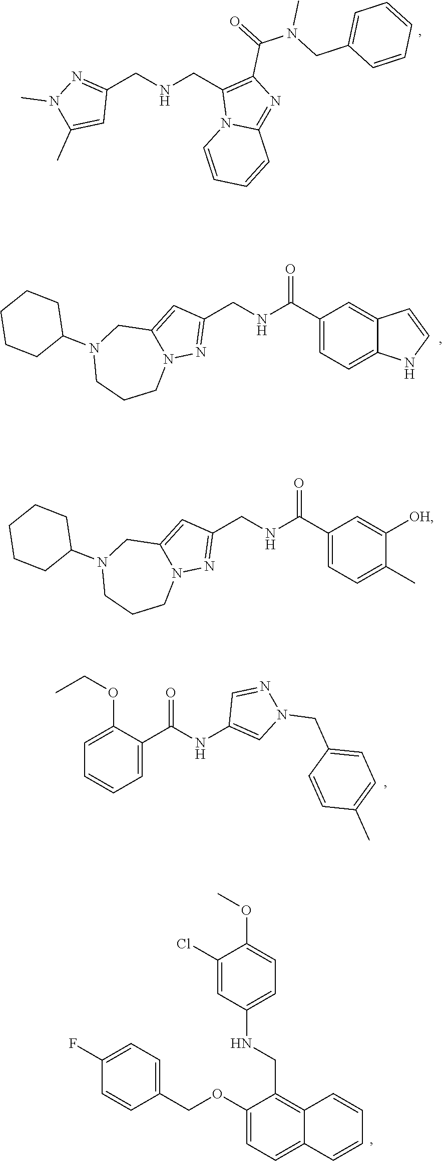

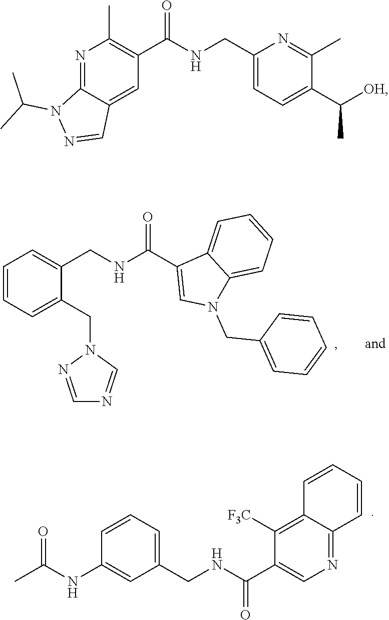 [SMILES: Cc1nc2c(cnn2C(C)C)cc1C(=O)NCc1ccc([C@H](C)O)c(C)n1 ; O=C(NCc1ccccc1Cn1cncn1)c1cn(Cc2ccccc2)c2ccccc12 ; CC(=O)Nc1cccc(CNC(=O)c2cnc3ccccc3c2C(F)(F)F)c1]

In certain embodiments, the Tip60-dependent and/or Tip60-overexpressing is at least one selected from the group consisting of breast cancer, prostate cancer, colorectal cancer, lung cancer, ovarian cancer, glioblastoma, hepatocellular carcinoma, gastric cancer, leukemia, melanoma, pancreatic cancer, bladder cancer, cervical cancer, esophageal cancer, head and neck squamous cell carcinoma, renal cell carcinoma, thyroid cancer, non-small cell lung cancer, endometrial cancer, and lymphoma.

In certain embodiments, the subject is further administered at least one additional agent suitable for the treatment, prevention, and/or amelioration of cancer.

### Pharmaceutical Compositions and Formulations

The disclosure provides pharmaceutical compositions comprising at least one compound of the disclosure or a salt or solvate thereof, which are useful to practice methods of the disclosure. Such a pharmaceutical composition may consist of at least one compound of the disclosure or a salt or solvate thereof, in a form suitable for administration to a subject, or the pharmaceutical composition may comprise at least one compound of the disclosure or a salt or solvate thereof, and one or more pharmaceutically acceptable carriers, one or more additional ingredients, or some combination of these. At least one compound of the disclosure may be present in the pharmaceutical composition in the form of a physiologically acceptable salt, such as in combination with a physiologically acceptable cation or anion, as is well known in the art.

In certain embodiments, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 1,000 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the disclosure may be suitably developed for nasal, inhalational, oral, rectal, vaginal, pleural, peritoneal, parenteral, topical, transdermal, pulmonary, intranasal, buccal, ophthalmic, epidural, intrathecal, intravenous or another route of administration. A composition useful within the methods of the disclosure may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the disclosure are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs.

In certain embodiments, the compositions of the disclosure are formulated using one or more pharmaceutically acceptable excipients or carriers. In certain embodiments, the pharmaceutical compositions of the disclosure comprise a therapeutically effective amount of at least one compound of the disclosure and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure include but are not limited to those selected from the group consisting of benzyl alcohol, sorbic acid, parabens, imidurea and combinations thereof. One such preservative is a combination of about 0.5% to 2.0% benzyl alcohol and 0.05% to 0.5% sorbic acid. The composition may include an antioxidant and a chelating agent which inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the disclosure may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the disclosure may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

Compounds of the disclosure for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the disclosure is from about 0.5 μg and about 5,000 mg. In some embodiments, a dose of a compound of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans) buccal, (trans) urethral, vaginal (e.g., trans- and perivaginally), (intra) nasal, and (trans) rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the disclosure may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present disclosure also includes a multi-layer tablet comprising a layer providing for the delayed release of one or more compounds useful within the methods of the disclosure, and a further layer providing for the immediate release of one or more compounds useful within the methods of the disclosure. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the disclosure which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the disclosure may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically- or naturally derived.

### Buccal Administration

A pharmaceutical composition of the disclosure may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the disclosure includes additional modifications of these and other formulations not described herein, but which are known to those of skill in the art.

### Rectal Administration

A pharmaceutical composition of the disclosure may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patent applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems:

In certain embodiments, the compositions and/or formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation. In certain embodiments of the disclosure, the compounds useful within the disclosure are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours. The term pulsatile release is used herein in its conventional sense to refer to a drug formulation that provides release of the drug in such a way as to produce pulsed plasma profiles of the drug after drug administration. The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present disclosure. However, they are in no way a limitation of the teachings or disclosure of the present disclosure as set forth herein.

### EXAMPLES

Various embodiments of the present application can be better understood by reference to the following Examples which are offered by way of illustration. The scope of the present application is not limited to the Examples given herein.

### Materials and Methods

### Fly Strains and Crosses

All fly lines were raised on standard yeast Drosophila medium (Applied Scientific Jazz Mix Drosophila Food, Thermo Fischer Scientific) at 25° C. with 12/12-h light/dark cycle. The pan-neuronal driver elav^{C155}-Gal4, transgenic Tip60-RNAi-mediated knock-down (UAS-Tip60 RNAi) and the transgenic UAS lines carrying human APP 695 isoform (UAS-APP) were obtained from Bloomington Drosophila Stock Center. For neural Tip60 knockdown and APP expression, the elav^{C155}-Gal4 driver line was crossed with UAS-Tip60RNAi or UAS-APP and the progeny was collected as Tip60 knockdown model (elav^{C155}-Gal4/UAS-Tip60 RNAi) or APP associated AD model (elav^{C155}-Gal4/UAS-APP). For wild-type control, the elav^{C155}-Gal4 driver line was crossed with itself. Model validation was performed by confirming reduced Tip60 mRNA levels in Tip60 RNAi model as compared to wildtype using real-time PCR.

### Drug Delivery

Compounds were synthesized by Enamine, Princeton Molecular and ChemBridge. The compounds were incorporated into the fly food and fed to the progeny larvae and flies as described previously. DMSO was used as a vehicle to prepare drug solutions. The fly food was prepared under standard conditions and aliquoted into individual vials. The drug solutions were then added to the fly vials with final DMSO concentration ≤0.01% and DMSO only was used as a control. The parent crosses were set in the drug food vials so that the resulting progeny can continue feeding on the drug since the early developmental stage.

### In Silico Ligand-Based Virtual Screening

A curated version of ZINC15 compound library of commercially available compounds was used for high-throughput virtual screening. For the substructure search, the SMILES (CCOCl═CC═CC═ClC(═O)NC2=CC(═C(C═C2)Cl)C(F)(F)F) for CTB compound (PubChem: 729859): N-[4-chloro-3-(trifluoromethyl)phenyl]-2-ethoxybenzamide were inserted in the query section. The top 100 compounds were exported as a .mol2 file that was used for protein-ligand docking on the GOLD software. GOLD software is widely used for virtual screening of protein-ligand interactions and lead optimization. For pharmacophore-based search, CTB was docked on Tip60's HAT domain (PDB:2ou2) and interactions were limited with filters H-bond=−0.2, distance 6 Å and energy=−0.5. A pharmacophore is defined as the ensemble of steric and electronic features that enable a compound to interact with a specific biological target and trigger a biological response, therefore isolating the overall “essence” of the structure-activity knowledge from a compound. Structurally different molecules with similar pharmacophore models can therefore be substituted for targeting the same target binding site as a strategy to enhance both specificity of the compound and the biological responses they trigger. Accordingly, pharmacophore-modeling is now considered an indispensable component of computer-aided drug design. Drug-like compounds were screened on a curated ZINC15 database with purchasable compounds using two pharmacophore modeling searches and results were combined and filtered for GOLD docking score of 78.

### In Silico Protein-Ligand Docking

All protein-ligand docking was performed using genetic algorithm methodology of GOLD docking software (Cambridge Crystallographic Data Centre). Twenty conformations were used for every ligand to sample the conformational space. The Acetyl-CoA binding pocket of Tip60's HAT domain (PDB:2ou2) was identified by using a 10 Å filter and was composed of the following residues: LEU354, GLU351, HIS274, ILE318, ASP273, PHE271, CYS317, SER355, LEU272, LEU357, SER361, THR320, GLY330, GLY328, LYS331, ARG326, GLN325, ARG327, and TYR324. A flexible docking option was used so both the protein and the ligands are treated flexible to optimize the fit. The protein-ligand complexes were scored using the default Gold score method available from the GOLD docking program.

### In Vitro Surface Plasma Resonance (SPR)

SPR protein-compound binding interactions were analyzed on Biacore 3000 biosensor (Global Cytiva Lifesciences) at 25° C. PBS-P (10 mM Phosphate, 150 mM NaCl, pH 7.4, 0.005% P-20) was used as the running buffer and a HAT-optimized binding buffer (50 mM HEPES, pH 7.4, 500 mM NaCl, 0.05% [v/v] Tween 20, 5 mM DTT, 10% [w/v] glycerol, 2% [v/v] DMSO) was used. A CM5 sensor chip was docked and derivatized by amine coupling to capture anti-GST antibody using freshly prepared 1:1 50 mM NHS (N-hydroxysuccinamide): 200 mM EDC (1-ethyl-3-(3-(dimethylamino) propyl) carbodiimide) followed by immobilization of GST-Tip60 protein (Abcam: ab2686960). Direct binding was determined by injecting ligands in duplicates at concentrations spanning 0.0078 μM to 50 μM with running buffer injections in between. DMSO corrections were performed as described previously. The corrected responses were transformed into % bound sites and plotted against the compound concentration. The concentration of Tip60 on the surface was estimated using [Tip60]=response (captured RU)/100*MW. To plot the % bound isotherm as a function of concentration, it is assumed that binding is 1:1 and the following equation is used: % A bound to B is 100AB/Bt=[Kd+At+Bt-SQRT((Kd+At+Bt)2−4*At*Bt)]/(2*Bt) where A=C and B=Tip60.

### In Vivo Locomotor Speed and Line Crossing Assays

Larval locomotion function was analyzed as previously described. The line crossing apparatus consisted of a petri dish containing 2.5% agar positioned on a 0.5 cm² grid paper. Wandering third instar of either sex larvae were collected from fly vials and rinsed with distilled water. The larvae were placed in the center of petri dish and allowed to acclimate for 1 minute. After initial acclimation, the larvae were recorded for 30 seconds on the plate. Larval speed (mm/s) was analyzed using Tracker video analysis and modeling tool as previously described. For the line crossing assay, the number of lines crossed by the head of a larvae in 30 s on a grid 0.5 cm×0.5 cm was recorded. Data from a minimum of 20 larvae were used per experiment.

### In Vitro Histone Acetylation Activity Assay

HAT assays were performed according to manufacturer's instructions in HAT Assay Kit (Active Motif 56100). Briefly, 375 ng Tip60 protein (Active motif: 81975) was incubated with 50 μM Histone H4 peptide, 50 μM Acetyl-CoA and indicated drug concentrations for 30 minutes at room temperature. After stopping the reaction and developing free sulfhydryl groups, absorbance was recorded at excitation 360-390 nm and emission 460-490 nm using Glomax fluorescence microplate reader. Specific enzyme activity (pmol/min/μg) was calculated by 1. Subtracting background fluorescence from that of assay samples to obtain arbitrary fluorescence units (AFU); and (2) Dividing the difference by the incubation time to obtain AFU/minute. 3. Dividing the AFU/minute by the slope of standard curve in AFU/pmol to obtain the rate in pmol/minute. 4. Dividing pmol/min by the amount of enzyme used to obtain final readings in pmol/minute/μg.

### Reverse Transcriptase Real Time PCR (RT-qPCR)

Total RNA was isolated from 40 staged third instar larval brains using the Quick-RNA Miniprep kit (Zymo research). cDNA was prepared using the SuperScript II reverse transcriptase kit (Invitrogen) according to the manufacturer's instructions with 1 μg of total RNA. Primers were designed using NCBI Primer-BLAST and the primer pair specificity was analyzed using the reference sequence database of Drosophila melanogaster (taxid: 7227). RT-qPCRs were performed in a 10 μL reaction volume containing cDNA, 1 μM Power SYBR Green PCR Master Mix (Applied Biosystems), and 10 μM forward and reverse primers. Primer sequences used are: Rp132 Forward: TGGTTTCCGGCAAGCTTCAA (SEQ ID NO:1), Reverse: TGTTGTCGATACCCTTGGGC (SEQ ID NO:2). Tip60 Forward: TCGCTATCTGGCAATACTGCTG (SEQ ID NO:3), Reverse: GTTAGGGCGGCCGTTGACT (SEQ ID NO:4). RT-qPCR was performed using an ABI 7500 Real-Time PCR system (Applied Biosystems) following the manufacturer's instructions. Fold change in mRNA expression was determined by the ooCt method relative to wildtype using Rp132 as housekeeping gene.

### Experimental Design and Statistical Analysis

All statistical analysis were performed using GraphPad Prism version 9.5.1 software package. For Tip60-RNAi model validation using RT-qPCR, unpaired Student's t-test was used to compare mRNA fold change between wildtype and Tip60-RNAi conditions with p<0.05. For locomotor speed assays, statistical significance between the wildtype vehicle, Tip60-RNAi vehicle and Tip60-RNAi with various drug concentrations was calculated using one-way ANOVA with Dunnett's multiple comparisons with p<0.05. For all experiments, at least 20 larvae were used as previously described in the literature.

### Example 1: General HAT Activators are Predicted to be with TIP60's HAT Domain

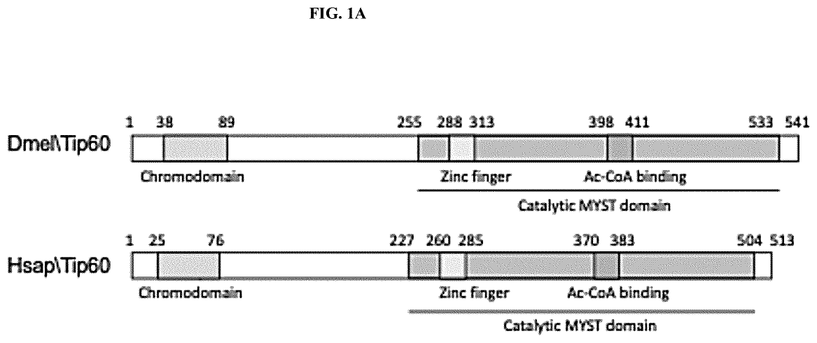

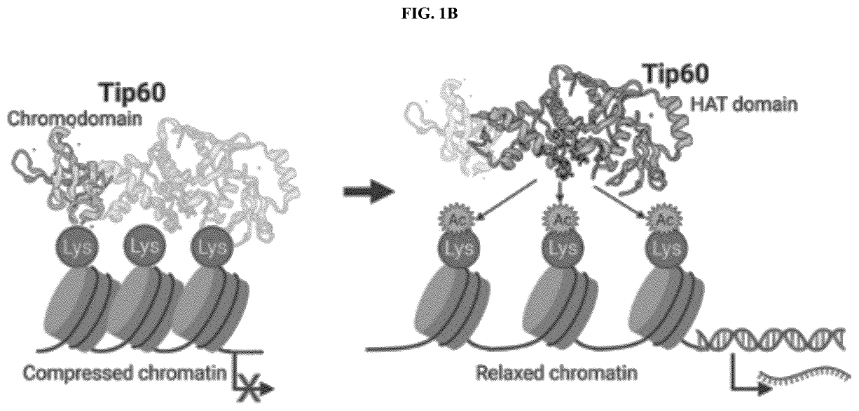

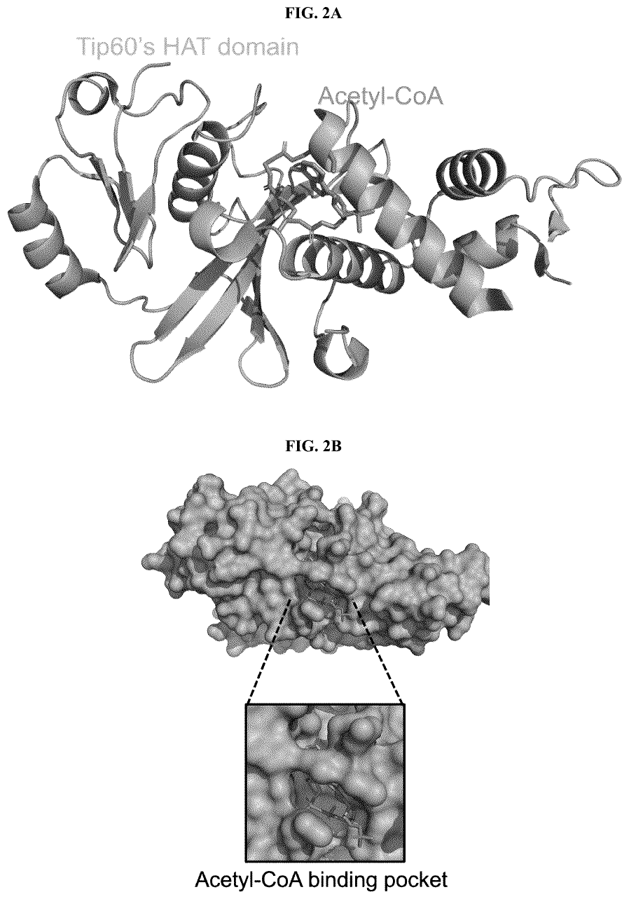

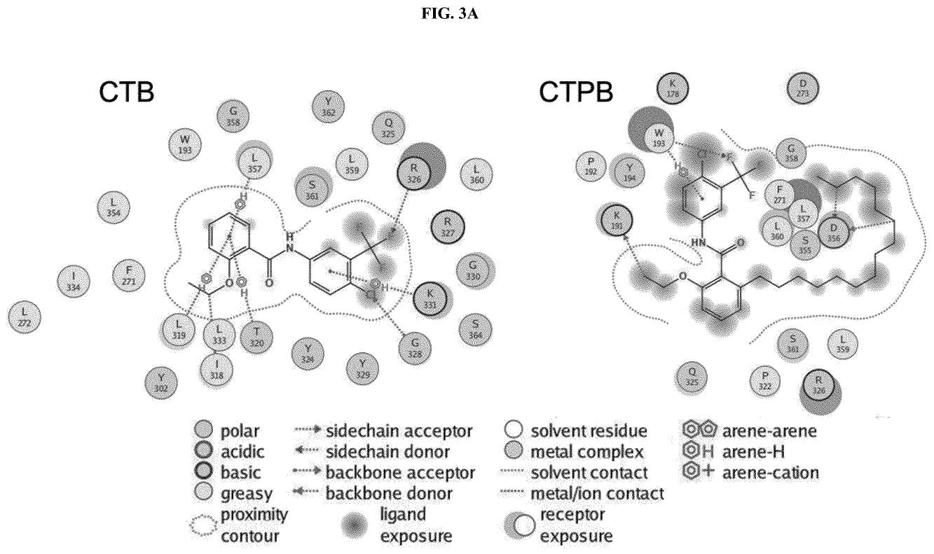

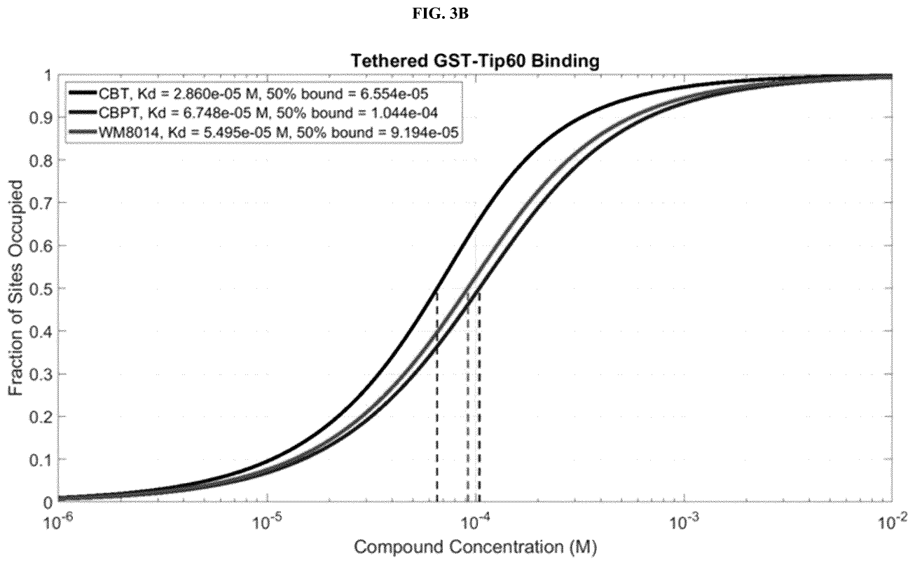

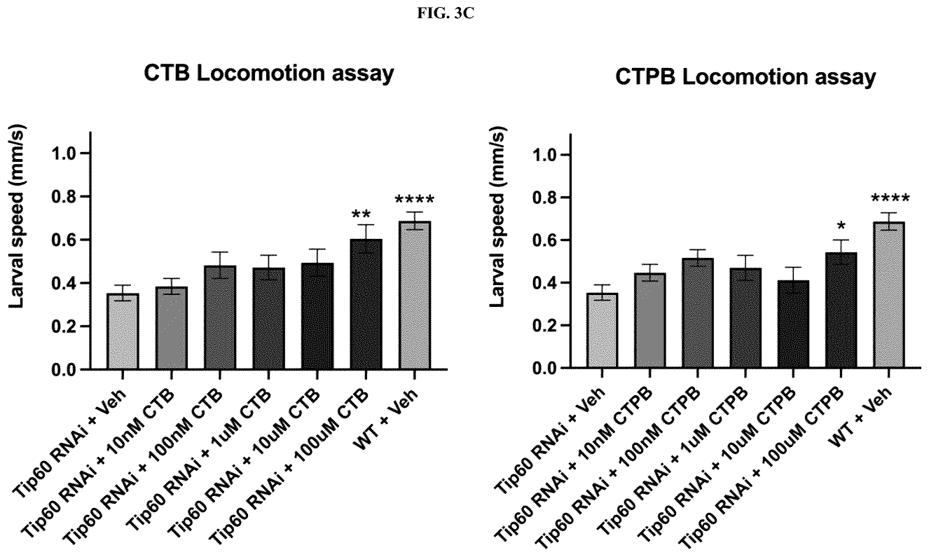

Since CTB and CTPB have previously been shown to activate the enzymatic activity of another HAT enzyme, p300, in in vitro experiments, these compounds were tested to see if they could be used as an initial lead to further modify for specific activation of Tip60's HAT domain. Both, CTB and CTPB are small molecule HAT activity modulators that are trifluoromethyl phenyl benzamides suggested to activate p300 acetyltransferase activity by inducing conformational changes in the enzyme structure. CTB (Chem ID: 729859) is N-[4-Chloro-3-(Trifluoromethyl) Phenyl]-2-Ethoxybenzamide with molecular formula C₁₆H₁₃ClF₃NO₂ and molecular weight MW: 343.73 g/mol, whereas CTPB (Chem ID: 10311918) is N-(4-Chloro-3-Trifluoromethyl-Phenyl)-2-Ethoxy-6-Pentadecyl-Benzamide with molecular formula C₃₁H₄₃ClF₃NO₂ and molecular weight 554.1 g/mol. To determine if these HAT activators also interact with Tip60's HAT domain, an in silico GOLD protein-ligand docking algorithm was used. Tip60 contains two major protein domains: an N-terminal chromodomain that recognizes methylated lysine residues on histone proteins and recruits Tip60 to target gene loci and a C-terminal catalytic MYST HAT domain that transfers the acetyl group from Acetyl-CoA to histone proteins for acetylation (FIGS. 1A-1B). The 3D crystal structure of Tip60's HAT domain (isoform 3) in complex with Acetyl coenzyme A in X-ray diffraction with resolution of 2.30 Å was obtained from Protein data bank (PDB:2ou2). The Acetyl-CoA binding site in the acetyltransferase domain of Tip60 was identified by filtering for residues within 10 Å (FIGS. 2A-2B). Upon docking, both CTB and CTPB were predicted to interact with Tip60's HAT domain with a favorable docking score of 52 and 59, respectively (FIG. 3A). Despite its slightly lower docking score, CTB displayed favorable characteristics over CTPB including its smaller size (343 vs. 554 Dalton) and lack of insoluble long hydrophobic side chain, along with trifluorides (—CF₃), all critical for blood-brain barrier (BBB) permeability, making CTB the favorable lead for Tip60-activator drug design.

### Example 2: CTB Directly Binds and Activates Tip60's HAT Function In Vitro and Prevents Tip60-Knockdown Mediated Functional Neuronal Locomotion Deficits In Vivo

Next, it was validated if CTB and CTPB directly bind with the Tip60 protein to exert functional effects. Surface plasma resonance (SPR) was used, which is an optical detection technique used to study label-free biomolecular interactions between a ligand (Tip60 protein) and analytes (candidate compounds dissolved in DMSO) in real-time (FIG. 3B). CTB, CTPB, and WM8014 (known Tip60 inhibitor control, positive control) were sequentially passed through a GST-Tip60 antibody captured biosensor chip in increasing drug concentrations and changes in intensity of reflected light were measured as calculated as % bound sites and were corrected for any non-specific binding to the reference surface (Tables 2A-2C). The SPR readings results were corrected for non-specific binding on reference surface and DMSO calibration so readings only account for direct Tip60-compound complexes on the experimental chip. All three compounds showed direct binding with Tip60 protein. CTB showed slightly higher binding affinity (KD=28.6 μM) than CTPB (KD=67.4 μM) and known Tip60 inhibitor WM8014 (KD=54.9 μM), where KD is the dissociation constant and is the concentration of ligand, which half the ligand binding sites on the protein are occupied in the system equilibrium. These results support the favorability of CTB as the lead compound for Tip60-activation drug design.

TABLE 2A
Surface plasma resonance (SPR) Tip60-compound
binding results for WM8014*

| Concentration | Subtracted | Corrected | % Bound |
|---|---|---|---|
| (μM) | response | Response | sites |
| 0.0078 | 8.58 | 0.126984 | 0.044713 |
| 0.023 | 7.78 | 0.115144 | 0.040544 |
| 0.07 | 7.42 | 0.109816 | 0.038668 |
| 0.21 | 7.24 | 0.107152 | 0.03773 |
| 0.63 | 7.51 | 0.111148 | 0.039137 |
| 1.85 | 8.17 | 0.120916 | 0.042576 |
| 5.56 | 11.34 | 0.167832 | 0.059096 |
| 17 | 22.255 | 0.329374 | 0.115977 |
| 50 | 61.705 | 0.913234 | 0.321561 |

*the subtracted response represents SPR reading for experimental conditions minus the background reading with GST-control protein reference. Corrected response represents correction for DMSO determined via 5 calibration solutions (running buffer with 2.5 ± 0.37% DMSO concentrations).

TABLE 2B
Surface plasma resonance (SPR) Tip60-compound
binding results for CTB*

| Concentration | Subtracted | Corrected | % Bound |
|---|---|---|---|
| (μM) | response | Response | sites |
| 0.0078 | 8.34 | 0.132606 | 0.052413 |
| 0.023 | 7.955 | 0.1264845 | 0.049994 |
| 0.07 | 7.83 | 0.124497 | 0.049208 |
| 0.21 | 7.885 | 0.1253715 | 0.049554 |
| 0.63 | 8.37 | 0.133083 | 0.052602 |
| 1.85 | 9.425 | 0.1498575 | 0.059232 |
| 5.56 | 12.615 | 0.2005785 | 0.07928 |
| 17 | 24.05 | 0.382395 | 0.151144 |
| 50 | 64.54 | 1.026186 | 0.405607 |

*the subtracted response represents SPR reading for experimental conditions minus the background reading with GST-control protein reference. Corrected response represents correction for DMSO determined via 5 calibration solutions (running buffer with 2.5 ± 0.37% DMSO concentrations).

TABLE 2C
Surface plasma resonance (SPR) Tip60-compound
binding results for CTPB*

| Concentration | Subtracted | Corrected | % Bound |
|---|---|---|---|
| (μM) | response | Response | sites |
| 0.0078 | 8.525 | 0.138105 | 0.03742683 |
| 0.023 | 8.62 | 0.139644 | 0.0378439 |
| 0.07 | 8.545 | 0.138429 | 0.03751463 |
| 0.21 | 8.575 | 0.138915 | 0.03764634 |
| 0.63 | 8.99 | 0.145638 | 0.03946829 |
| 1.85 | 10.085 | 0.163377 | 0.04427561 |
| 5.56 | 13.355 | 0.216351 | 0.05863171 |
| 17 | 25.005 | 0.405081 | 0.10977805 |
| 50 | 66.335 | 1.074627 | 0.29122683 |

*the subtracted response represents SPR reading for experimental conditions minus the background reading with GST-control protein reference. Corrected response represents correction for DMSO determined via 5 calibration solutions (running buffer with 2.5 ± 0.37% DMSO concentrations).

Next, it was tested if these compounds exert functional effects on Tip60 in vivo. Drosophila is a robust effective model to functionally test efficacy of pharmacological drugs and has been widely used for drug screening in various neurological disorders. Using the Gal4-UAS targeted gene expression system, expression of UAS-Tip60 RNAi can be modulated in all neurons using the pan-neuronal elav-Gal4 driver line. The resulting progeny with elav-Gal4; UAS-Tip60 RNAi genotype is referred to as a Tip60 RNAi knockdown model. The elav-Gal4 driver line alone serves as the wildtype control in these experiments. Tip60 mRNA levels are significantly reduced in the Tip60-RNAi model as compared to wild-type using RT-qPCR (p=0.0234, unpaired Student's t-test). Additionally, in line with previous results, Tip60 knockdown resulted in reduced larval locomotion as compared to wild-type (p<0.0001, one-way ANOVA with Dunnett's multiple comparisons), enabling the use of this model to test the functional efficacy of compounds to rescue the neuronal locomotion phenotype caused by Tip60 reduction.

Larvae were reared on food either with compounds (CTB, CTPB) or without compounds (DMSO vehicle) using standard published protocol and a speed assay was used to assess improvement in locomotion defects caused by Tip60 reduction (FIG. 3C). Treatment of larvae with both, CTB and CTPB, showed significant rescue for locomotor ability with CTB (p=0.0046) showing more robust therapeutic effects than CTPB (p=0.0290, one-way ANOVA with Dunnett's multiple comparisons. In summary, the direct binding of CTB to Tip60 and activation of its HAT activity in vitro along with prevention of Tip60-knockdown associated locomotor function in vivo further underscore favorability of CTB as a lead compound suitable for Tip60-activator drug design.

### Example 3: Design and Synthesis of Tip60 Modulators Using CTB Substructure-Based Virtual Screening

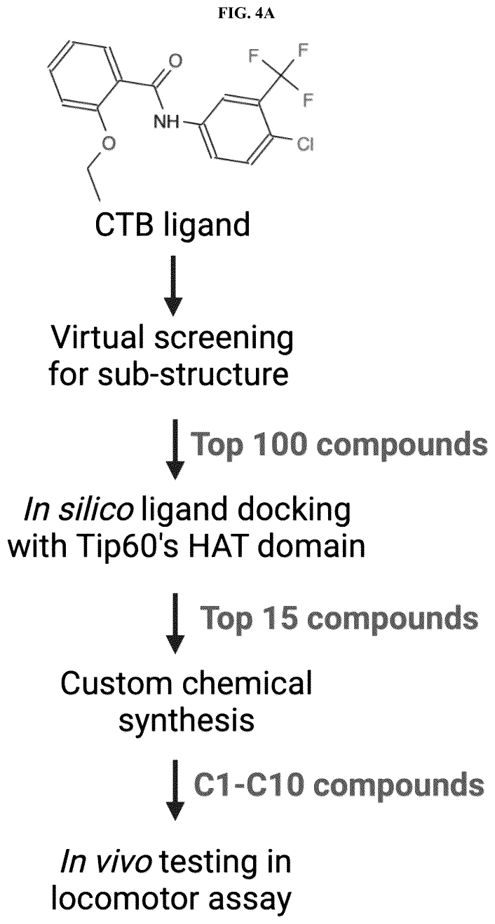

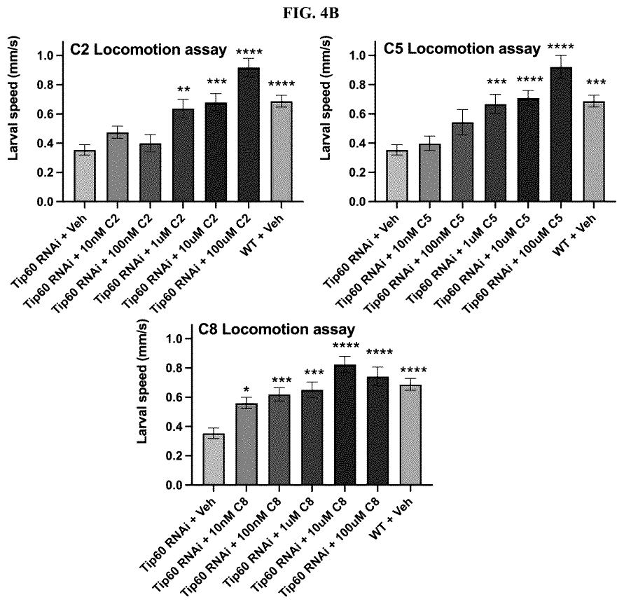

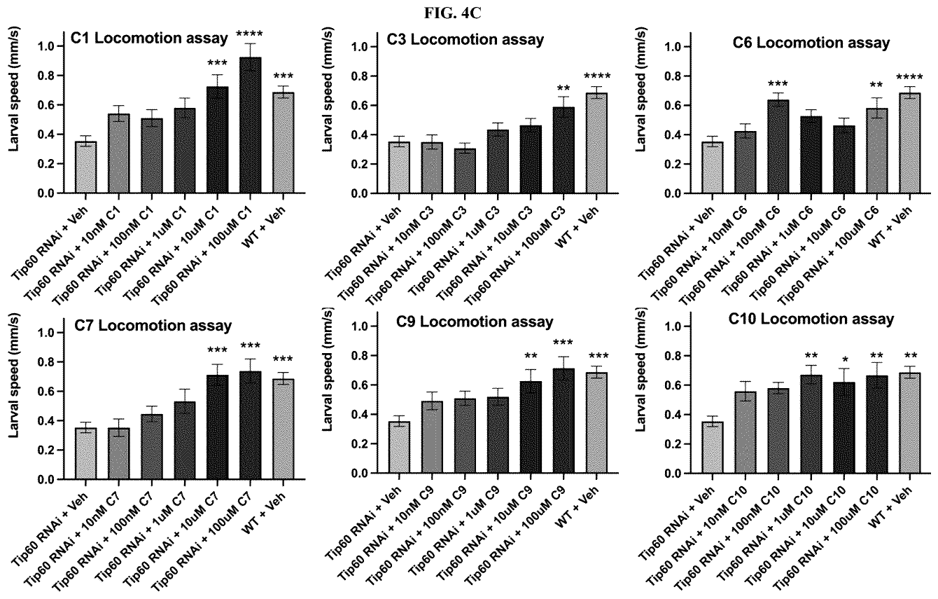

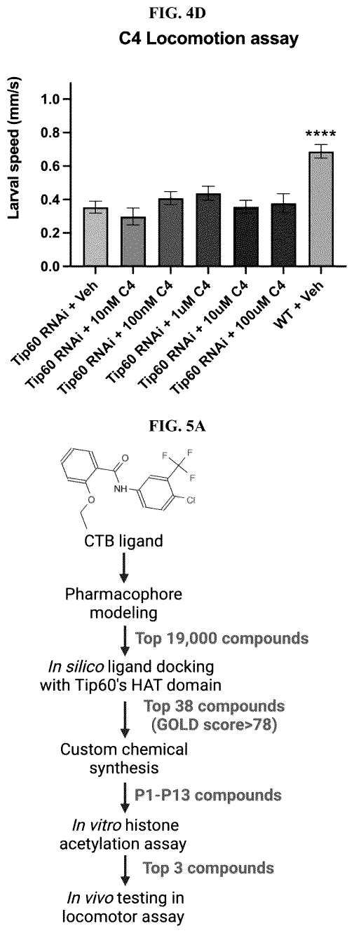

Proceeding with top performer CTB, a sub-structure search was performed to identify analogs with better specificity for Tip60. Importantly, since fluorides are essential for BBB permeability and the relative positioning of trifluorides (—CF₃) with the chloride (—Cl) in CTB are essential for HAT activation, retention of these halogens in potential Tip60-specific HAT activator design was ensured. A ligand substructure-based virtual screening approach was carried out using the commercially available ZINC15 chemical database with over 230 million compounds (FIG. 4A). Virtual screening to find compounds that have a similar substructure as CTB, additional beneficial chemical modifications to promote Tip60 binding specificity. First, CTB structure (SMILES: CCOCl═CC═CC═ClC(═O)NC2=CC(═C(C═C2)Cl)C(F)(F)F) was used as a query in the ZINC15 compound library. Structures of the top 100 compound hits with similar substructure were obtained and individually docked with Tip60's HAT domain using the GOLD protein-ligand docking algorithm. Each protein-compound interaction is individually scored using GOLD total docking score that is a summation of individual scores obtained via ligand atom contributions and their interaction types. These 100 compounds ranged in docking scores from 43 to 68, with higher score correlating with favorable protein-ligand binding energy. Of note, CTB ranked 57^{th} in the best docking scores, suggesting more than half of the identified compounds are predicted to have better Tip60 binding than CTB. Based on the GOLD docking scores, the top fifteen compounds with scores ranging from 58 to 68 were selected for further analysis (Table 3). These compounds favor binding to the same Tip60 binding pocket as CTB but have additional favorable functional groups that may aid in increased Tip60 binding affinity. The synthesis of ten out of these top fifteen compounds was chemically feasible and successfully synthesized by Enamine using on-demand building blocks. These compounds are now referred to as C1-C10 and were next tested for their functional efficacy (Table 3).

TABLE 3
C1-C10 compounds show better binding prediction
with Tip60's HAT domain as compared to CTB*

| | GOLD | PLP | H bond | CHO |
|---|---|---|---|---|
| Compound | score | score | score | score |
| C1 | 68.18 | −67.14 | 1.6 | 0 |
| C2 | 62.75 | −52.03 | 4.56 | 0 |
| C3 | 61.55 | −60.19 | 1.78 | 0 |
| C4 | 60.7 | −54.42 | 2.46 | 0 |
| C5 | 60.65 | −51.34 | 4.79 | 0 |
| C6 | 60 | −62.31 | 0.12 | 0 |
| C7 | 60 | −55.98 | 1.95 | 0 |
| C8 | 59.08 | −47.94 | 4.43 | 0 |
| C9 | 58.95 | −55.69 | 2 | 0 |
| C10 | 58.16 | −46.49 | 4.1 | 0 |
| CTB | 52.44 | −54.17 | 0 | 0 |

| | Metal | DE Clash | DE Tors | Int Cor |
|---|---|---|---|---|
| Compound | Score | Score | Score | Score |
| C1 | 0 | 3.62 | 3.04 | 5.65 |
| C2 | 0 | 0 | 2.71 | 2.47 |
| C3 | 0 | 0 | 4.62 | 5.25 |
| C4 | 0 | 0.12 | 3.07 | 5.16 |
| C5 | 0 | 2.19 | 3.68 | 4.48 |
| C6 | 0 | 0 | 2.34 | 1.51 |
| C7 | 0 | 1.91 | 2.72 | 4.44 |
| C8 | 0 | 0 | 2.94 | 3.29 |
| C9 | 0 | 0 | 3.27 | 3.81 |
| C10 | 0 | 1.38 | 2.35 | 5.29 |
| CTB | 0 | 5.1 | 2.08 | 7.53 |

*GOLD protein-ligand docking was utilized to individually dock compounds on Tip60's HAT domain and score each interaction. GOLD score represents the total docking score with higher score correlated with protein-ligand binding energy and is a summation of individual scores obtained via ligand atom contributions and their interaction types. PLP: pairwise additive scoring function of acceptor-donor pairs and/or lipophilic potentials. H-bond: Protein- ligand H-bonding. CHO: weak CH . . . OH- bonds. Metal: Metal-binding contribution. DE clash: Protein-ligand clash penalty. DE tors: Internal ligand torsional strain penalty to the PLP value. Internal correlation: Internal ligand energy offset.

### Example 4: Substructure-Based Tip60 Modulators Prevent Tip60-Knockdown Mediated Neuronal Locomotion Defects In Vivo

To further validate and filter our ten lead compounds for their functional effectiveness, each of these compounds was tested using an in vivo functional Drosophila larval locomotor speed assay to see if they are able to rescue locomotor defects observed in our Tip60-RNAi knockdown model. As described elsewhere herein, larvae were reared on food either with compounds dissolved in DMSO or without compounds (DMSO vehicle) using a standard published protocol and a speed assay was used to assess improvement in locomotion defects caused by Tip60 reduction. Remarkably, treatment with most compounds significantly rescued larval locomotor ability over a wide range of concentrations. Out of the ten compounds, C2, C5 and C8 were the highest performers that showed the most significant rescue even at lower drug concentrations as compared to CTB (FIG. 4B). Compounds C1, C3, C6, C7, C9 and C10 were designated as low-performing compounds since they performed either similar to CTB or slightly better in rescuing locomotor defects (FIG. 4C). Unlike the other compounds, C4 did not show an effect even at high concentrations, possibly due to its insolubility or inability to target and/or activate Tip60 (FIG. 4D). Together, these results show that several substructure-based candidate compounds for Tip60 HAT activation fully rescue locomotor deficits in our Tip60 knockdown model.

### Example 5: Design and Synthesis of Tip60 Modulators Using CTB Pharmacophore Based Virtual Screening

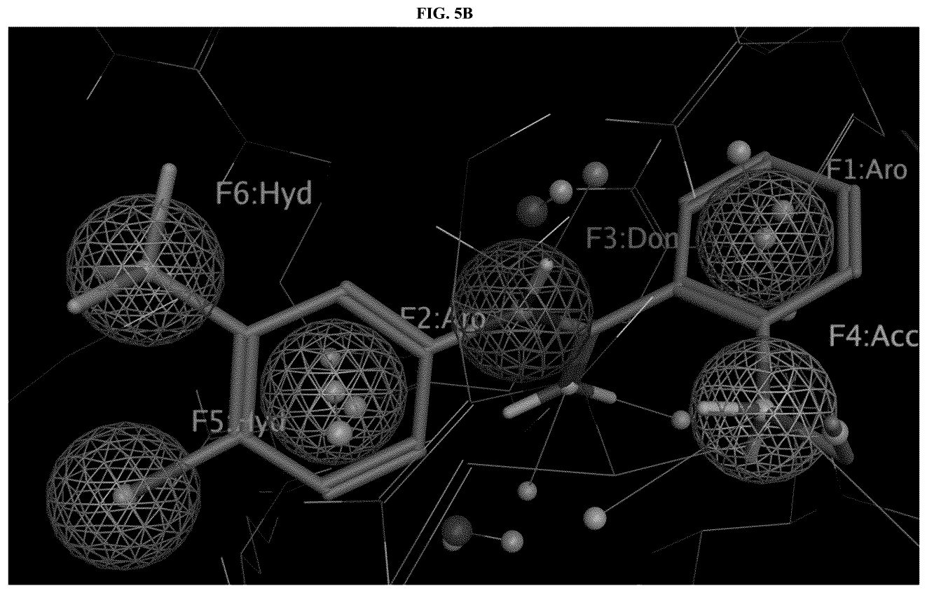

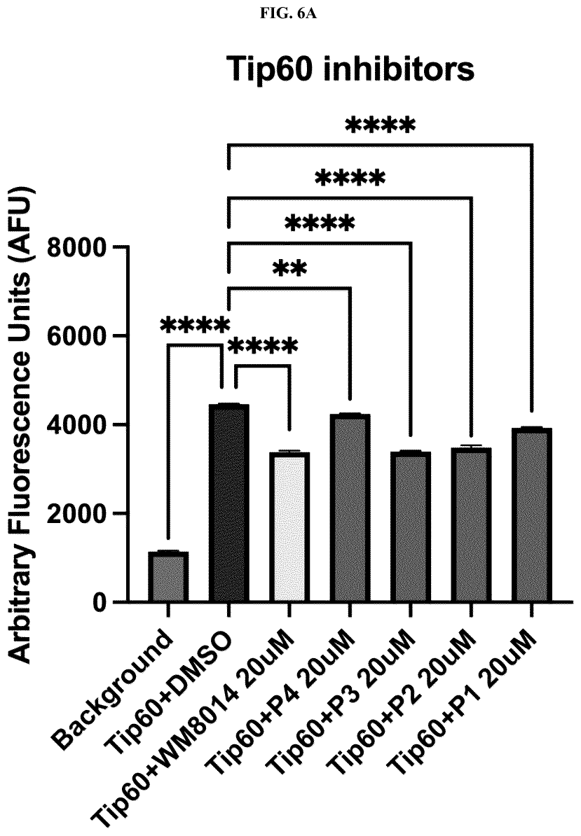

Although candidate compounds generated via substructure-based virtual screening show promise in activating Tip60's HAT domain, they are limited by their potential non-specificity towards p300 HAT as well as their marginal increase in predicted docking to Tip60's HAT domain (docking scores of C1-C10 range from 58 to 68 as compared to docking score 52 for CTB). Therefore, a novel pharmacophore based virtual screening approach was employed for Tip60 HAT activator drug design. Major pharmacophore modeling features include H-bond acceptor, H-bond donor, anionic, cationic, hydrophobic, and aromatic groups. To generate a pharmacophore model of CTB, the pharmacophore editor feature in Molecular Operating Environment (Chemical Computing Group ULC) was employed. A total of 6 pharmacophore features were identified in CTB: two aromatic rings (F1, F2), one hydrogen donor site (F3), one hydrogen acceptor site (F4), and two hydrophobic sites (F5, F6) (FIG. 5B). Using these pharmacophore features, a standard pharmacophore-based virtual screening pipeline was employed for identification of Tip60 HAT activators (FIG. 5A). In the first pharmacophore search (PP1), all CTB pharmacophore features were retained except only one of the aromatic rings was included, resulting in ˜5800 hits. In the second pharmacophore search (PP2), all CTB pharmacophore features were retained except only one of the aromatic rings was included and only one feature out of aromatic ring F1, hydrogen acceptor F4 or hydrophobic site F6 was included, resulting in 13,147 hits. Combining these two screens, a total of approximately 19,000 hits were obtained that were then individually docked on Tip60's HAT domain using GOLD docking software. Using a filter cutoff of Gold docking score 78 or higher, 38 compounds were identified that were triaged based on high docking score, ease of synthesis and similarity in core structure. 13 of these compounds, with docking scores ranging from 78 to 81, were custom synthesized by Enamine, Princeton Molecular and ChemBridge. These compounds favor binding to the same Tip60 binding pocket as CTB but have considerably higher docking scores than CTB (78 to 81 vs 52), indicating a higher binding affinity and specificity towards Tip60's HAT domain. These compounds are now referred to as P1-P13 and were next tested for their functional effectiveness.

TABLE 4
P1-P13 compounds show better binding prediction
with Tip60's HAT domain as compared to CTB*

| | GOLD score | GOLD score |
|---|---|---|
| Cmpd | Pharmacophore 1 | Pharmacophore 2 |
| P13 | 79.43 | 79.05 |
| P12 | 81.5 | 81.19 |
| P11 | 80.66 | — |
| P10 | 80.25 | — |
| P9 | 81.7 | 81.87 |
| P8 | 80.86 | 80.06 |
| P7 | 78.62 | — |
| P6 | 78.51 | 78.58 |
| P5 | — | 78.9 |
| P4 | — | 80.67 |
| P3 | — | 79.38 |
| P2 | — | 79.12 |
| P1 | 78.6 | 78.59 |

*GOLD protein-ligand docking was utilized to individually dock compounds identified via Pharmacophore 1 or Pharmacophore 2 screen on Tip60's HAT domain and score each interaction. GOLD score represents the total docking score with higher score correlated with protein-ligand binding energy and is a summation of individual scores obtained via ligand atom contributions and their interaction types.

### Example 6: Pharmacophore Based Tip60 Modulators Act as Direct Tip60 Enzyme Activators or Inhibitors in Histone Acetylation Assay In Vitro

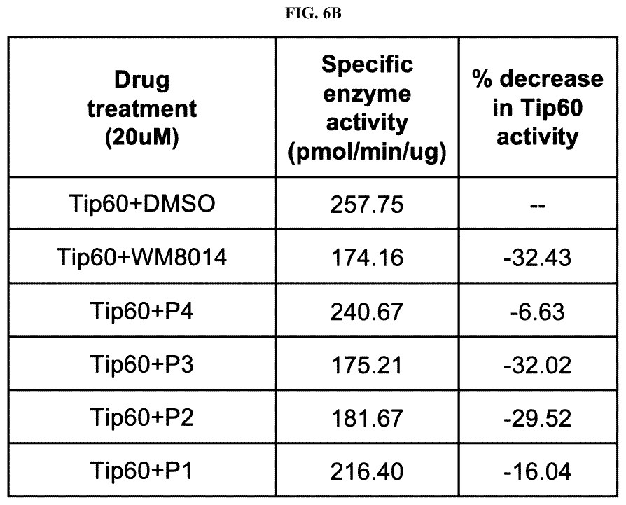

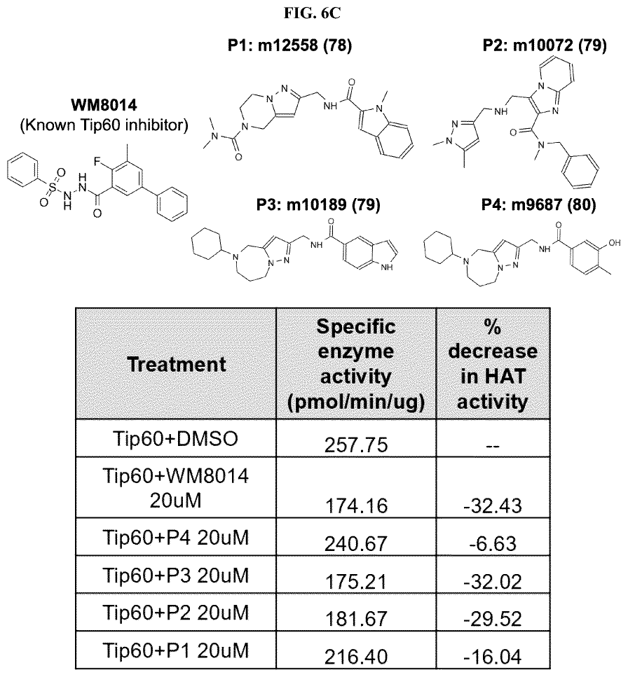

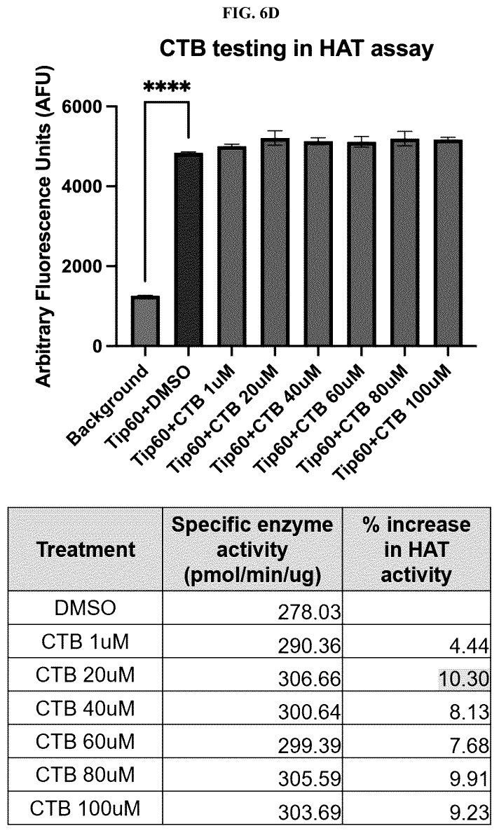

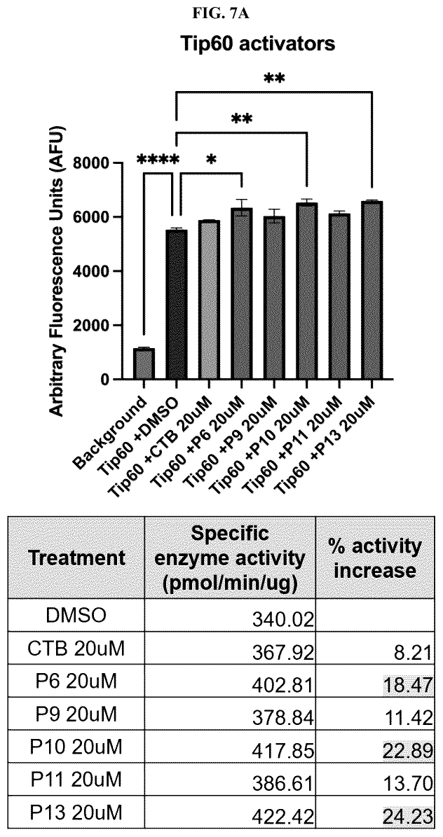

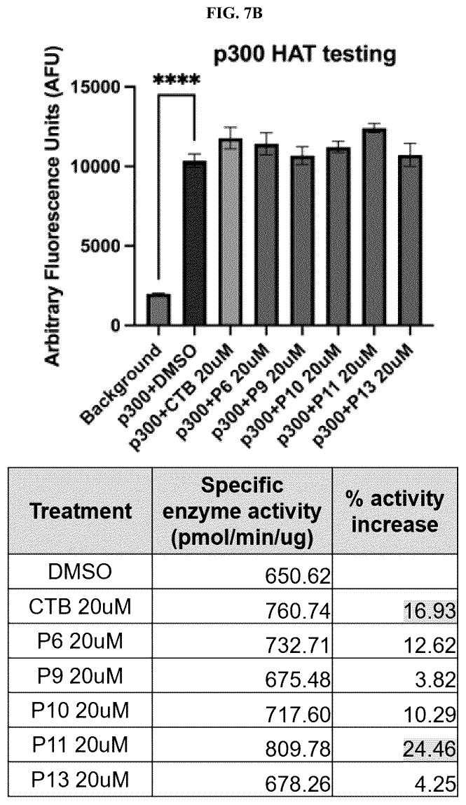

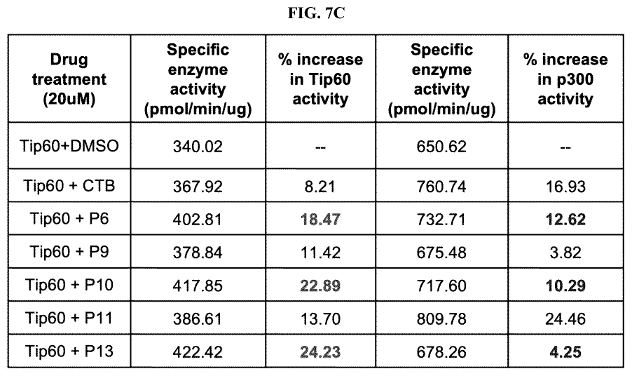

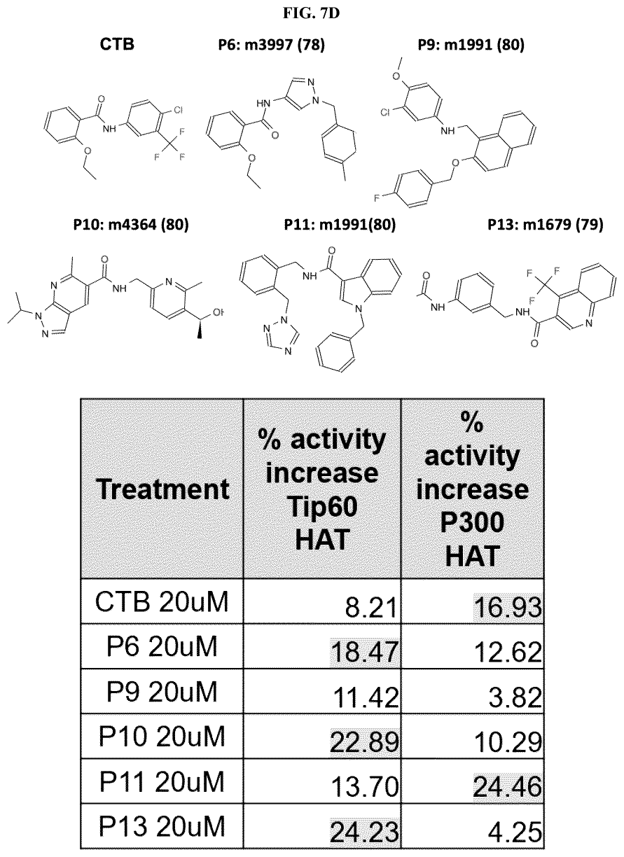

To further validate and filter our 13 pharmacophore-based lead compounds, each of these compounds were tested for enhancement or reduction in Tip60's HAT activity using an in vitro histone acetylation setup. This screen identified 4 out of these 13 compounds (P1-P4) that reduce Tip60's HAT activity and therefore result in significantly lower fluorescence reading in the presence of these compounds (FIGS. 6A-6C). Of note, P3 demonstrated a similar level of inhibition as the known Tip60 inhibitor, WM8014, suggesting P3 as a novel potent inhibitor of Tip60's HAT activity. Remarkably, 5 out of the 13 compounds (P6, 9, 10, 11, 13) enhanced Tip60's HAT activity thereby resulting in a significantly higher fluorescence in the presence of these compounds, with P6, P10 and P13 compounds displaying the highest level of Tip60 HAT activation (FIG. 7A and FIGS. 7C-7D). To test compound specificity, the in vitro HAT assay was repeated, this time using purified p300 HAT and it was found that as all P compounds did not significantly enhance p300 activity, with P10 and P13 showing highest specificity for Tip60 over p300 (FIGS. 7B-7C). Of note, and as predicted based on Tip60 docking scores, P compounds identified as activators displayed more robust enhancement for Tip60 HAT activity when compared to our lead CTB compound. In summary, several pharmacophore-based candidate compounds for Tip60 modulation show direct targeting to Tip60 enzyme that either enhances or reduces its HAT activity in vitro.

### Example 7: Top Pharmocophore Designed Tip60 Activators P6, P10, P13 Prevent Neuronal Locomotion Deficits in a Tip60-Knockdown Model In Vivo

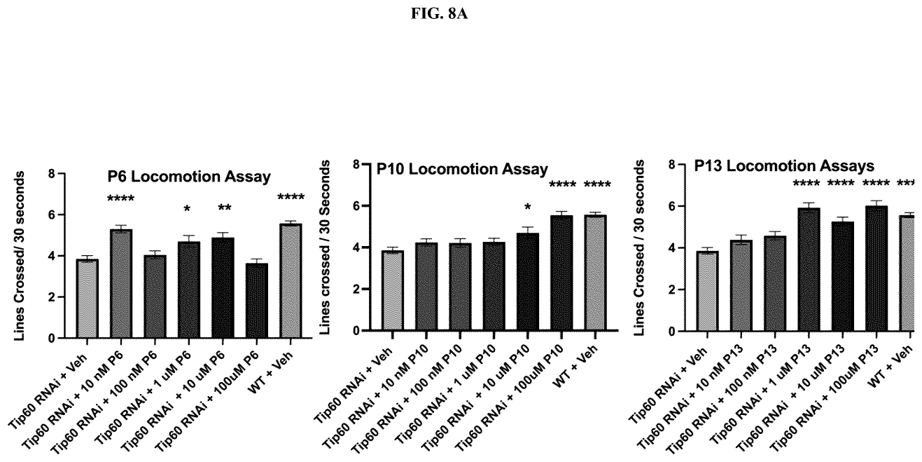

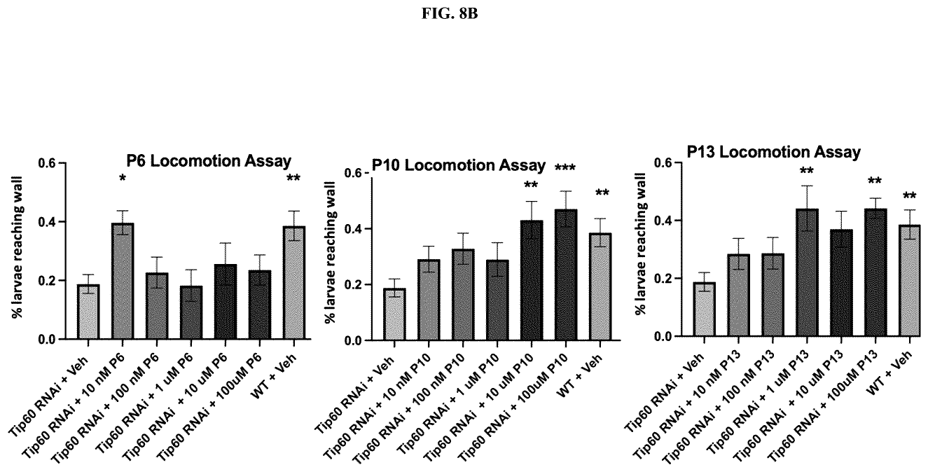

The highest performing Tip60 activators (P6, 10, 13) with robust Tip60 HAT activation and specificity were tested in the Drosophila larval locomotor assay to assess their functional efficacy in rescuing Tip60-RNAi knockdown mediated locomotion defects, in vivo. Larvae were reared on food either with compounds dissolved in DMSO or vehicle (DMSO) only to test their ability to reduce locomotor defects caused by Tip60 reduction (FIG. 8A). Remarkably, all three P compounds rescued Tip60 knockdown induced locomotion deficits, with P13 as the best performer in restoring locomotor activity to wild-type levels even at 1 μM drug concentration. It was also quantitated whether compound treatment enabled larvae the locomotion ability required to complete crossing the plate and reach the wall (FIG. 8B). Consistent with line crossing results, P13 improved the performance of larvae in reaching the wall than wild-type at a 1 μM drug concentration. Of note, P6 performed irregularly in that it showed inconsistent rescue results amongst larvae tested within a given pool and lacked a dosage dependency trend, rescuing at lower concentrations while showing no effect at higher concentrations. This may be due to P6 not getting absorbed readily into some larvae, and/or insolubility of the drug in the food at high concentrations. Together, these results highlight the therapeutic potential of the pharmacophore based compounds.

### Example 8: Top Pharmocophore-Based Tip60 Activators P10 and P13 Protect Against Functional Neuronal Deficits and Lethality in a Well Characterized Drosophila Alzheimer's Disease Model

Early pre-clinical and late AD neurodegenerative stages in humans are conserved both epigenetically and pathologically in a well characterized and widely used Drosophila AD associated amyloid precursor protein (APP) model that inducibly expresses full length human APP pan-neuronally. Third-instar APP larvae that model early stage AD neurodegeneration display deficits in cognitive ability, synaptic plasticity, axonal transport and outgrowth, and apoptotic neuronal cell death in the brain. AD larval brains also exhibit robust genome-wide early alterations that include genome-wide enhanced HDAC2 and reduced Tip60 levels and chromatin binding at critical neuroplasticity genes that trigger transcriptional dysregulation. These alterations are accompanied by locomotion impairment caused by these neuronal defects. Adult flies that model late stages of AD are characterized by reduced Tip60 and increased HDAC2 levels, plaques in the brain, cognitive deficits, and lethality. Such characteristics make APP Drosophila a powerful AD model to test the functional effectiveness of therapeutic drugs in both early and late stages of AD neurodegenerative progression, in vivo.

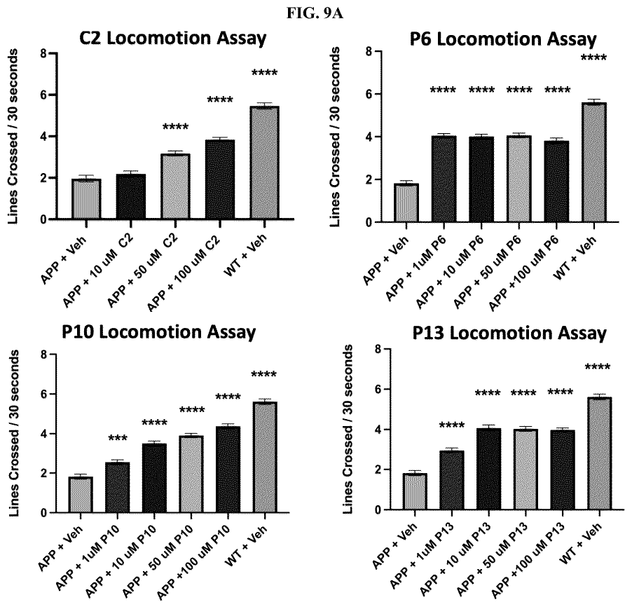

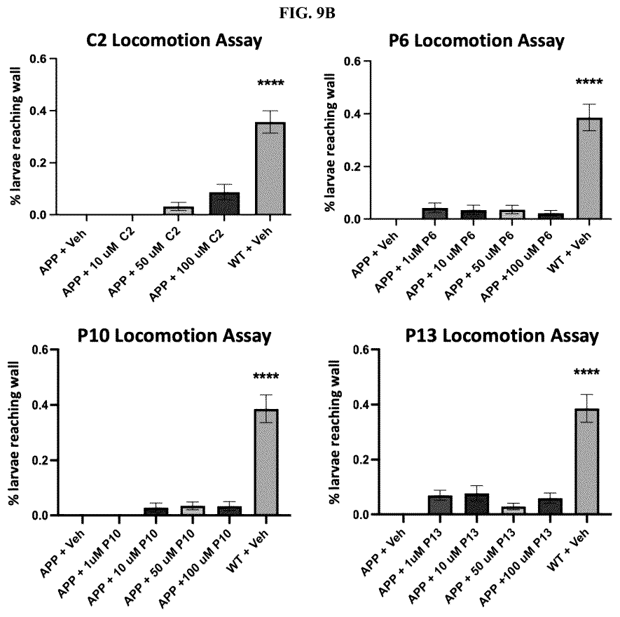

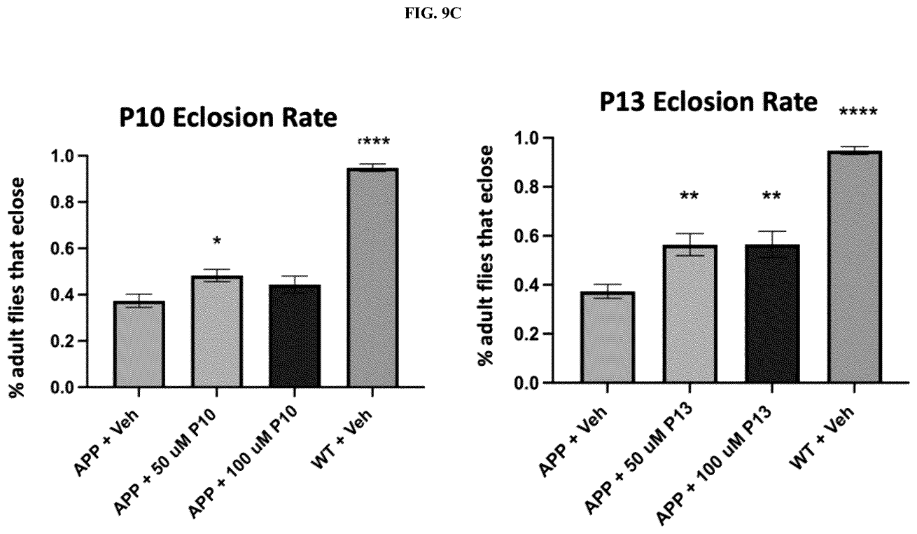

Next, the therapeutic effectiveness of the three top pharmacophore based compounds (P6, P10, P13) in protecting against both early and late stages of AD associated neurodegeneration was investigated. To do this, each compound's functional effectiveness in rescuing the severe AD associated locomotion defects in APP larvae that are indicative of early neuronal based deficits was assessed in vivo. Larvae were reared on food either with compounds dissolved in DMSO or vehicle (DMSO) only control using a standard published protocol and a line crossing assay was used to monitor locomotor function as previously described. Each of the three compounds tested significantly rescued larval locomotor ability over a wide range of concentrations (FIGS. 9A-9B) with all three compounds showing partial yet significant rescue effects even at 1 uM concentration.

Again, although P6 showed high rescue overall, its action was inconsistent as in a given testing pool, some larvae would exhibit rescue while others did not and no dose dependency was observed. The P10 and P13 compound's functional effectiveness in rescuing AD associated lethality that occurs as a culmination of later stage neurodgenerative progression was assessed using an eclosion assay (FIG. 9C). Briefly, larvae were reared on food either with compounds dissolved in DMSO or without compounds (DMSO vehicle) and the number of flies that eclosed from their pupal cases over a 7 day period were counted. Remarkably, both compounds showed significant rescue of lethality with P13 showing more robust effects. In summary, pharmacophore based Tip60 activators P10 and P13 showed consistent and robust rescue of both AD associated early stage severe locomotion deficits and later stage lethality, underscoring their therapeutic potential and effectiveness in the treatment of AD.

Sequence Listing

SEQ ID NO: 1 (Rpl32 Forward)
TGGTTTCCGGCAAGCTTCAA
SEQ ID NO: 2 (Rpl32 Reverse)
TGTTGTCGATACCCTTGGGC
SEQ ID NO: 3 (Tip60 Forward)
TCGCTATCTGGCAATACTGCTG
SEQ ID NO: 4 (Tip60 Reverse)
GTTAGGGCGGCCGTTGACT

### Enumerated Embodiments

The following exemplary embodiments are provided, the numbering of which is not to be construed as designating levels of importance:

Embodiment 1 provides a method of treating, preventing, and/or ameliorating a neurodegenerative disease, the method comprising administering to a subject in need thereof a therapeutically effective amount of at least one Tip60 activator or a pharmaceutically acceptable salt thereof.

Embodiment 2 provides the method of Embodiment 1, wherein the neurodegenerative disease is at least one selected from the group consisting of Alzheimer's Disease, Parkinson's Disease, Huntington's Disease, Amyotrophic Lateral Sclerosis (ALS), Frontotemporal Dementia, Multiple System Atrophy, Progressive Supranuclear Palsy, Corticobasal Degeneration, Lewy Body Dementia, Friedreich's Ataxia, Spinocerebellar Ataxia, Spinal Muscular Atrophy, Prion Diseases (such as Creutzfeldt-Jakob Disease), Wernicke-Korsakoff Syndrome, Kennedy's Disease (Spinal Bulbar Muscular Atrophy), Wilson's Disease, Niemann-Pick Disease, and Gerstmann-Sträussler-Scheinker Syndrome (GSS).

Embodiment 3 provides the method of Embodiment 1 or 2, wherein the neurodegenerative disease is at least one selected from the group consisting of Alzheimer's Disease, Parkinson's Disease, Huntington's Disease, Amyotrophic Lateral Sclerosis (ALS).

Embodiment 4 provides the method of any one of Embodiments 1-3, wherein the subject is further administered at least one additional agent suitable for the treatment, prevention, and/or amelioration of a neurodegenerative disease.

Embodiment 5 provides the method of any one of Embodiments 1-4, wherein the Tip60 activator is a compound of Formula (I) or a pharmaceutically acceptable salt thereof:

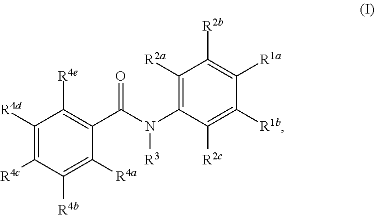

wherein:
R^{1a}, R^{1b}, and R^{1e} are each independently selected from the group consisting of H, optionally substituted C₁-C₆ alkyl, halogen, CN, and NO₂;
R^{1c} is halogen;
R^{1d} is C₁-C₃ haloalkyl;
R² is selected from the group consisting of H and optionally substituted C₁-C₆ alkyl;
R^{3a} and R^{3e} are each independently selected from the group consisting of optionally substituted C₁-C₁₂ alkoxy and optionally substituted C₁-C₂₀ alkyl; and
R^{3b}, R^{3c}, and R^{3d} are each independently selected from the group consisting of H, optionally substituted C₁-C₆ alkyl, halogen, CN, and NO₂.

Embodiment 6 provides the method of Embodiment 5, wherein at least one of the following applies:
(a) R^{1a}, R^{1b}, and R^{1e} are each independently H;
(b) R^{1c} is Cl;
(c) R^{1d} is CF₃; and
(d) R² is H.

Embodiment 7 provides the method of Embodiment 5 or 6, wherein R^{3c} is selected from the group consisting of OEt, OiPr,

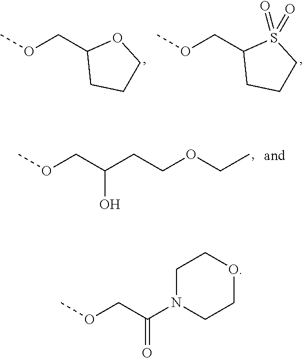

Embodiment 8 provides the method of any one of Embodiments 5-7, wherein R^{3e} is selected from the group consisting of H, (CH₂)₁₄CH₃, and O(CH₂)₂N(CH₃)₂.

Embodiment 9 provides the method of any one of Embodiments 5-8, wherein the compound of Formula (I) is selected from the group consisting of:

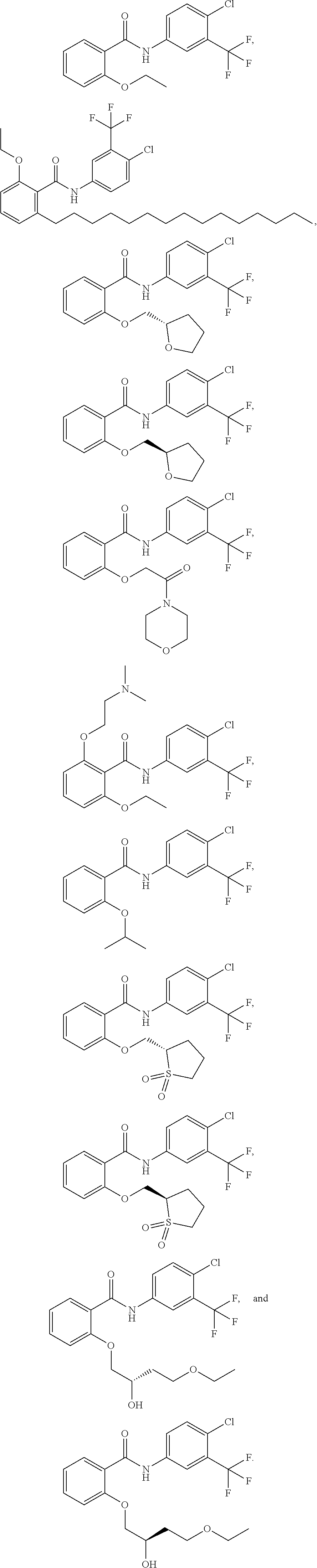

Embodiment 10 provides the method of any one of Embodiments 1-4, wherein the Tip60 activator is a compound of Formula (II) or a pharmaceutically acceptable salt thereof:

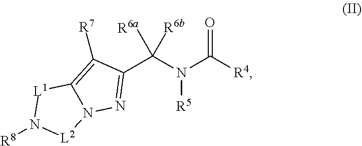

wherein:
R⁴ is selected from the group consisting of optionally substituted C₂-C₈ heteroaryl and C₆-C₁₀ aryl;
R⁵ is selected from the group consisting of H and optionally substituted C₁-C₆ alkyl;
R^{6a} and R^{6b} are each independently selected from the group consisting of H and optionally substituted C₁-C₆ alkyl;
R⁷ is selected from the group consisting of H, optionally substituted C₁-C₆ alkyl, halogen, CN, and NO₂;
R⁸ is selected from the group consisting of H, optionally substituted C₁-C₆ alkyl, optionally substituted C₃-C₈ cycloalkyl, optionally substituted C₂-C₈ heterocycloalkyl, and C(═O)N(R^{A})(R^{B});
L¹ is —[C(R^{9a})(R^{9b})]₁₋₂—;
L² is —[C(R^{10a})(R^{10b})]₁₋₂—;
each occurrence of R^{9a} and R^{9b} is independently selected from the group consisting of H and optionally substituted C₁-C₆ alkyl;
each occurrence of R^{10a} and R^{10b} is independently selected from the group consisting of H and optionally substituted C₁-C₆ alkyl; and
R^{A} and R^{B} are each independently selected from the group consisting of H and optionally substituted C₁-C₆ alkyl, or R^{A} and R^{B} combine with the N atom to which they are bound to form an optionally substituted C₂-C₈ heterocycloalkyl.

Embodiment 11 provides the method of Embodiment 10, wherein at least one of the following applies:
(a) R⁴ is selected from the group consisting of

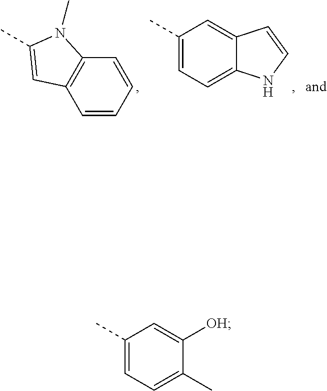

(b) R⁵ is H;
(c) R^{6c} and R^{6b} are each independently H;
(d) R⁷ is H;
(e) R⁸ is selected from the group consisting of cyclohexyl and C(═O)N(CH₃)₂;
(f) L¹ is —CH₂-;
(g) L² is selected from the group consisting of —(CH₂)₂— and —(CH₂)₃—.

Embodiment 12 provides the method of Embodiment 10 or 11, wherein the compound of Formula (II) is selected from the group consisting of:

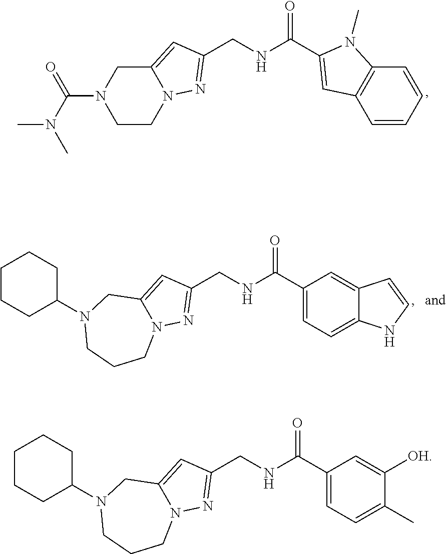 [SMILES: CN(C)C(=O)N1CCn2nc(CNC(=O)c3cc4ccccc4n3C)cc2C1 ; O=C(NCc1cc2n(n1)CCCN(C1CCCCC1)C2)c1ccc2[nH]ccc2c1 ; Cc1ccc(C(=O)NCc2cc3n(n2)CCCN(C2CCCCC2)C3)cc1O]

Embodiment 13 provides the method of any one of Embodiments 1-12, wherein the Tip60 activator is selected from the group consisting of:

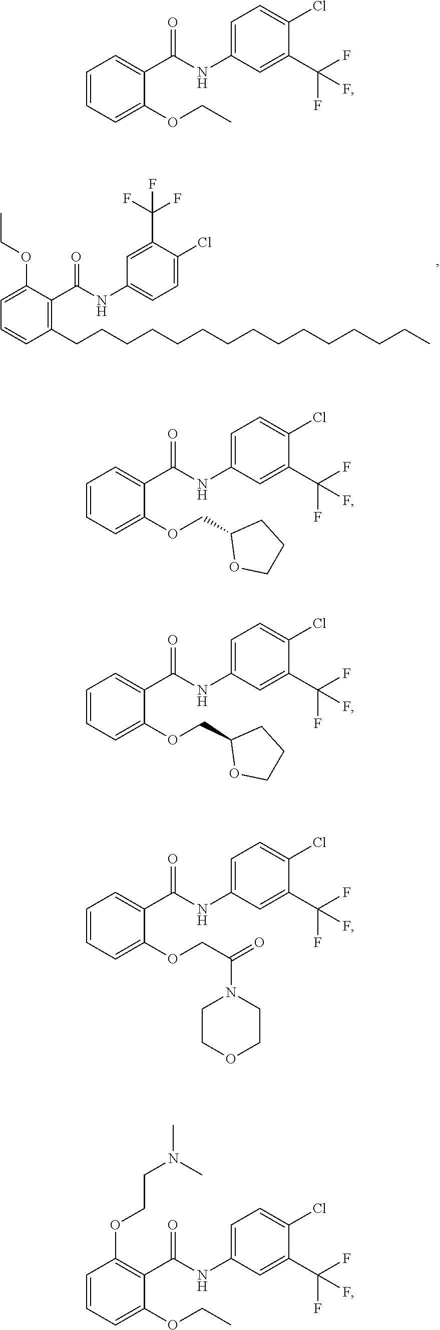 [SMILES: CCOc1ccccc1C(=O)Nc1ccc(Cl)c(C(F)(F)F)c1 ; CCCCCCCCCCCCCCCc1cccc(OCC)c1C(=O)Nc1ccc(Cl)c(C(F)(F)F)c1 ; O=C(Nc1ccc(Cl)c(C(F)(F)F)c1)c1ccccc1OC[C@@H]1CCCO1 ; O=C(Nc1ccc(Cl)c(C(F)(F)F)c1)c1ccccc1OC[C@H]1CCCO1 ; O=C(Nc1ccc(Cl)c(C(F)(F)F)c1)c1ccccc1OCC(=O)N1CCOCC1 ; CCOc1cccc(OCCN(C)C)c1C(=O)Nc1ccc(Cl)c(C(F)(F)F)c1]

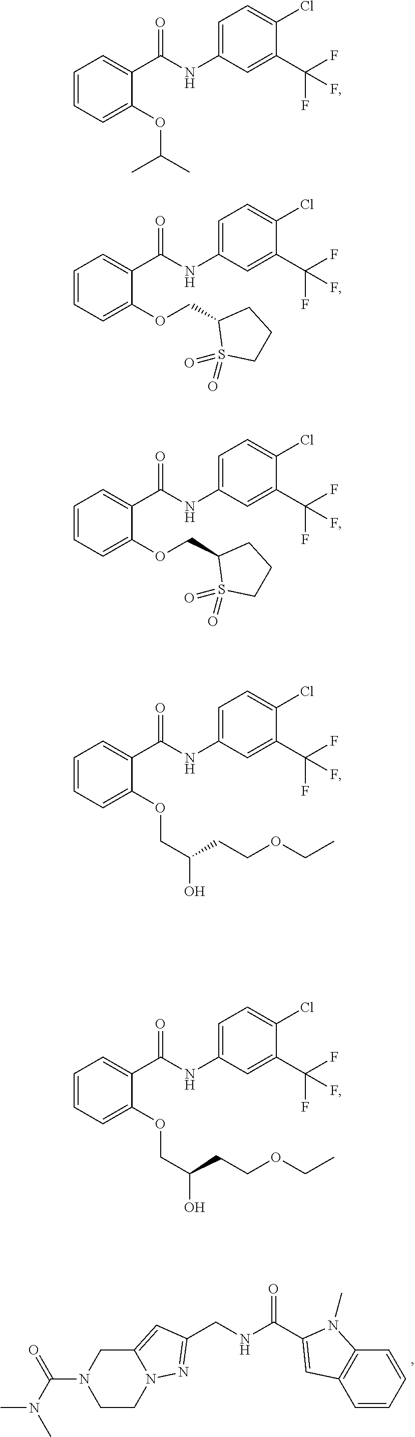

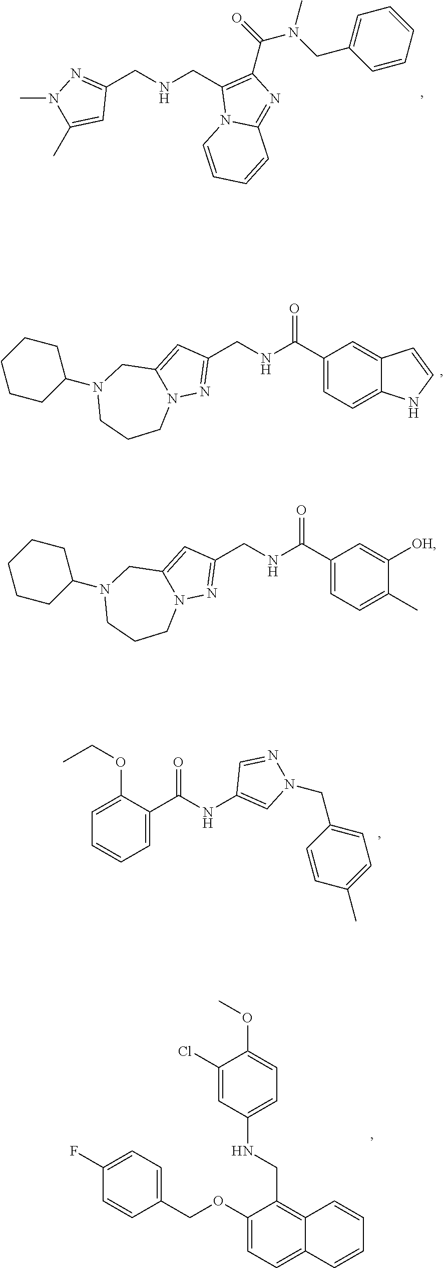 [SMILES: Cc1cc(CNCc2c(C(=O)N(C)Cc3ccccc3)nc3ccccn23)nn1C ; O=C(NCc1cc2n(n1)CCCN(C1CCCCC1)C2)c1ccc2[nH]ccc2c1 ; Cc1ccc(C(=O)NCc2cc3n(n2)CCCN(C2CCCCC2)C3)cc1O ; CCOc1ccccc1C(=O)Nc1cnn(Cc2ccc(C)cc2)c1 ; COc1ccc(NCc2c(OCc3ccc(F)cc3)ccc3ccccc23)cc1Cl]

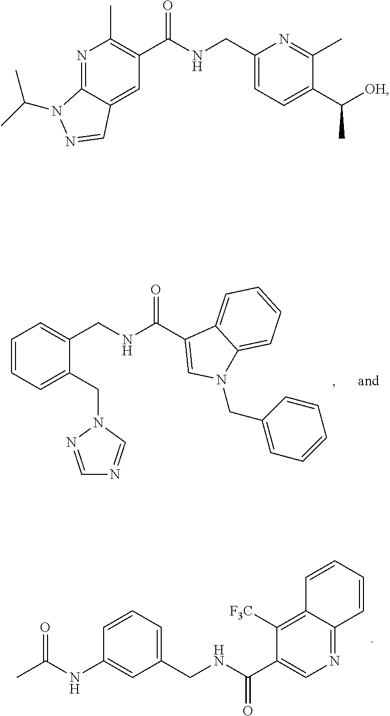 [SMILES: Cc1nc2c(cnn2C(C)C)cc1C(=O)NCc1ccc([C@H](C)O)c(C)n1 ; O=C(NCc1ccccc1Cn1cncn1)c1cn(Cc2ccccc2)c2ccccc12 ; CC(=O)Nc1cccc(CNC(=O)c2cnc3ccccc3c2C(F)(F)F)c1]

Embodiment 14 provides a method of treating, preventing, and/or ameliorating a Tip60-dependent and/or Tip60-overexpressing cancer, the method comprising administering to a subject in need thereof a therapeutically effective amount of a Tip60 inhibitor or a pharmaceutically acceptable salt thereof.

Embodiment 15 provides the method of Embodiment 14, wherein the Tip60 activator is a compound of Formula (I) or a pharmaceutically acceptable salt thereof:

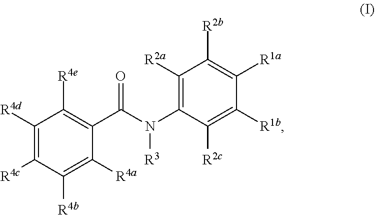

wherein:
R^{1a}, R^{1b}, and R^{1e} are each independently selected from the group consisting of H, optionally substituted C₁-C₆ alkyl, halogen, CN, and NO₂;
R^{1c} is halogen;
R^{1d} is C₁-C₃ haloalkyl;
R² is selected from the group consisting of H and optionally substituted C₁-C₆ alkyl;
R^{3a} and R^{3e} are each independently selected from the group consisting of optionally substituted C₁-C₁₂ alkoxy and optionally substituted C₁-C₂₀ alkyl; and
R^{3b}, R^{3c}, and R^{3d} are each independently selected from the group consisting of H, optionally substituted C₁-C₆ alkyl, halogen, CN, and NO₂.

Embodiment 16 provides the method of Embodiment 14, wherein the Tip60 activator is a compound of Formula (II) or a pharmaceutically acceptable salt thereof:

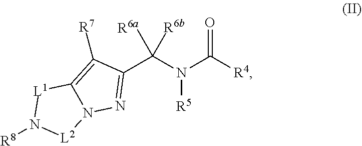

wherein:
R⁴ is selected from the group consisting of optionally substituted C₂-C₈ heteroaryl and C₆-C₁₀ aryl;
R⁵ is selected from the group consisting of H and optionally substituted C₁-C₆ alkyl;
R^{6a} and R^{6b} are each independently selected from the group consisting of H and optionally substituted C₁-C₆ alkyl;
R⁷ is selected from the group consisting of H, optionally substituted C₁-C₆ alkyl, halogen, CN, and NO₂;
R⁸ is selected from the group consisting of H, optionally substituted C₁-C₆ alkyl, optionally substituted C₃-C₈ cycloalkyl, optionally substituted C₂-C₈ heterocycloalkyl, and C(═O)N(R^{A})(R^{B});
L¹ is —[C(R^{9a})(R^{9b})]₁₋₂—;
L² is —[C(R^{10a})(R^{10b})]₁₋₂—;
each occurrence of R^{9c} and R^{9b} is independently selected from the group consisting of H and optionally substituted C₁-C₆ alkyl;
each occurrence of R^{10a} and R^{10b} is independently selected from the group consisting of H and optionally substituted C₁-C₆ alkyl; and
R^{A} and R^{B} are each independently selected from the group consisting of H and optionally substituted C₁-C₆ alkyl, or R^{A} and R^{B} combine with the N atom to which they are bound to form an optionally substituted C₂-C₈ heterocycloalkyl.

Embodiment 17 provides the method of Embodiment 14, wherein the Tip60 activator is selected from the group consisting of:

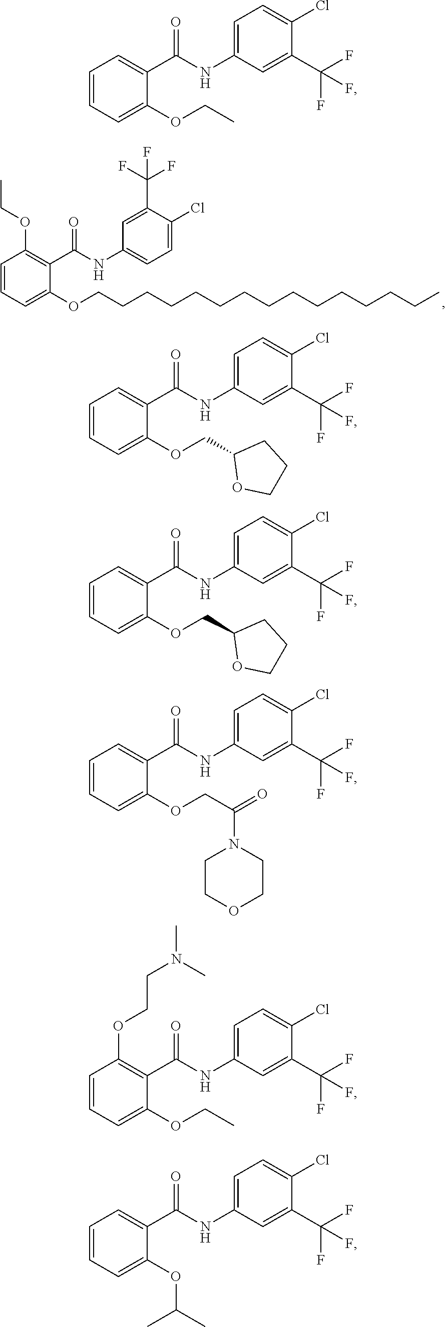

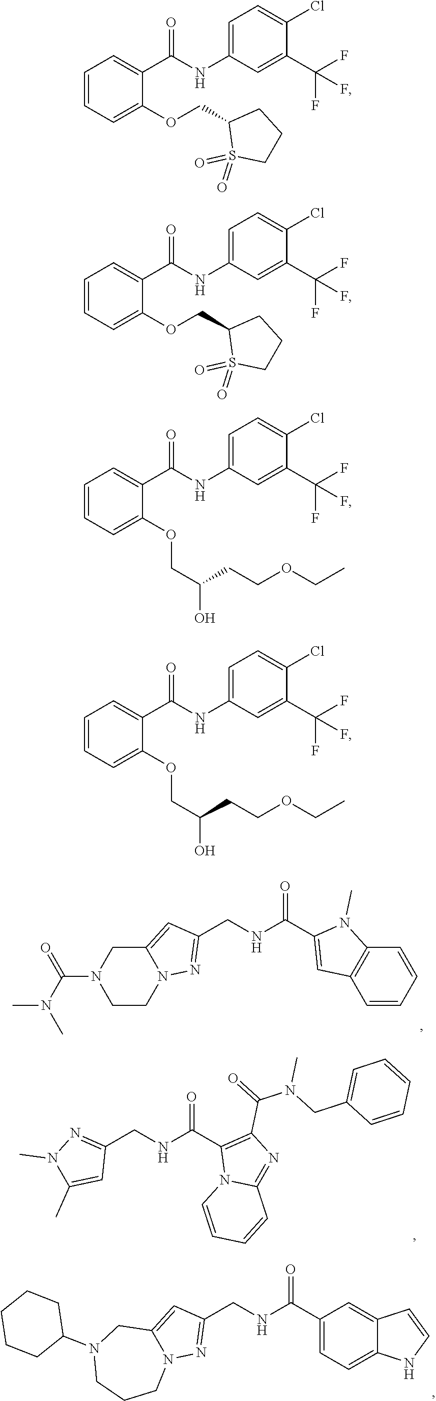

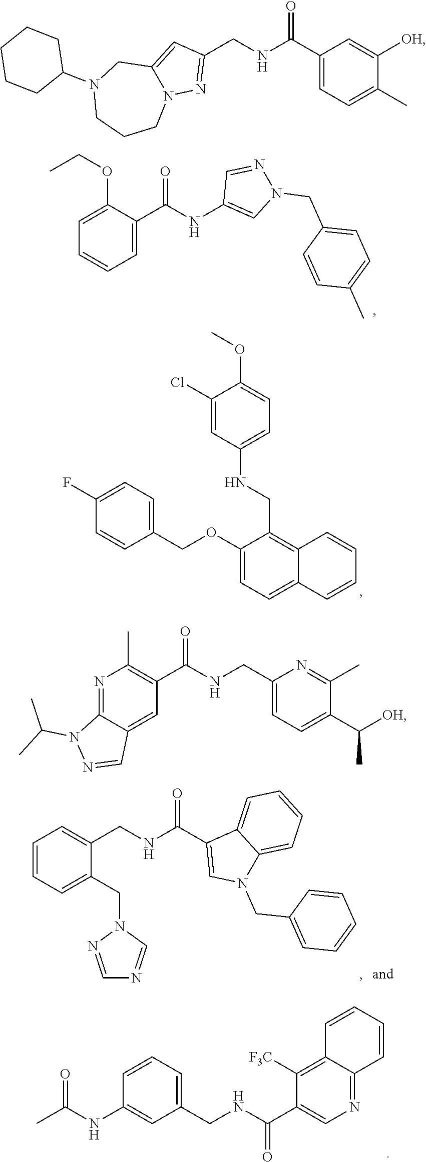

Embodiment 18 provides method of any one of Embodiments 14-17, wherein the Tip60-dependent and/or Tip60-overexpressing is at least one selected from the group consisting of breast cancer, prostate cancer, colorectal cancer, lung cancer, ovarian cancer, glioblastoma, hepatocellular carcinoma, gastric cancer, leukemia, melanoma, pancreatic cancer, bladder cancer, cervical cancer, esophageal cancer, head and neck squamous cell carcinoma, renal cell carcinoma, thyroid cancer, non-small cell lung cancer, endometrial cancer, and lymphoma.

Embodiment 19 provides method of any one of Embodiments 14-18, wherein the subject is further administered at least one additional agent suitable for the treatment, prevention, and/or amelioration of cancer.

Embodiment 20 provides a pharmaceutical composition comprising a pharmaceutically acceptable carrier and at least one compound selected from the group consisting of:

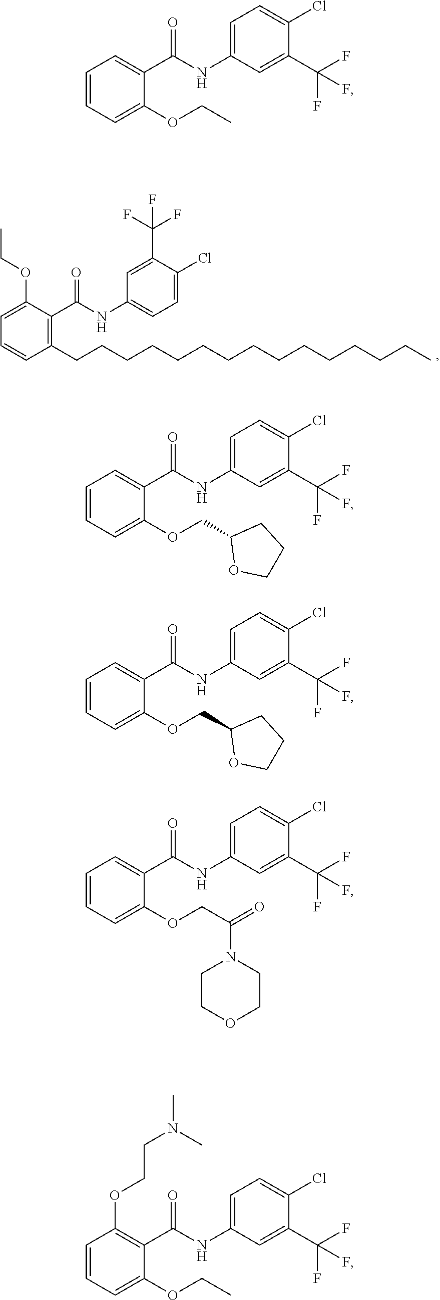

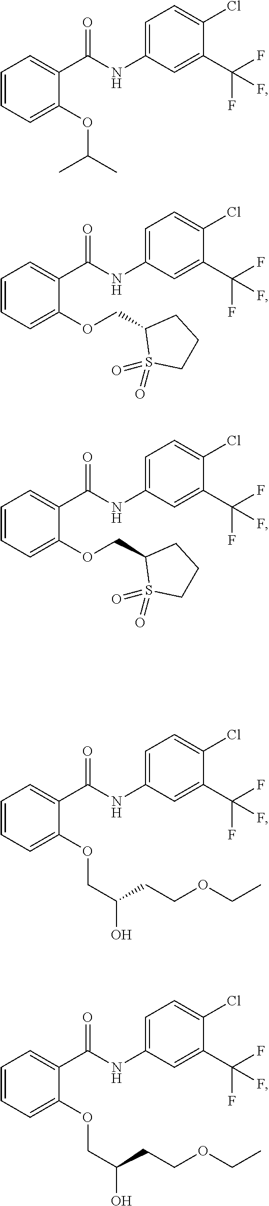 [SMILES: CC(C)Oc1ccccc1C(=O)Nc1ccc(Cl)c(C(F)(F)F)c1 ; O=C(Nc1ccc(Cl)c(C(F)(F)F)c1)c1ccccc1OC[C@@H]1CCCS1(=O)=O ; O=C(Nc1ccc(Cl)c(C(F)(F)F)c1)c1ccccc1OC[C@H]1CCCS1(=O)=O ; CCOCC[C@H](O)COc1ccccc1C(=O)Nc1ccc(Cl)c(C(F)(F)F)c1 ; CCOCC[C@@H](O)COc1ccccc1C(=O)Nc1ccc(Cl)c(C(F)(F)F)c1]

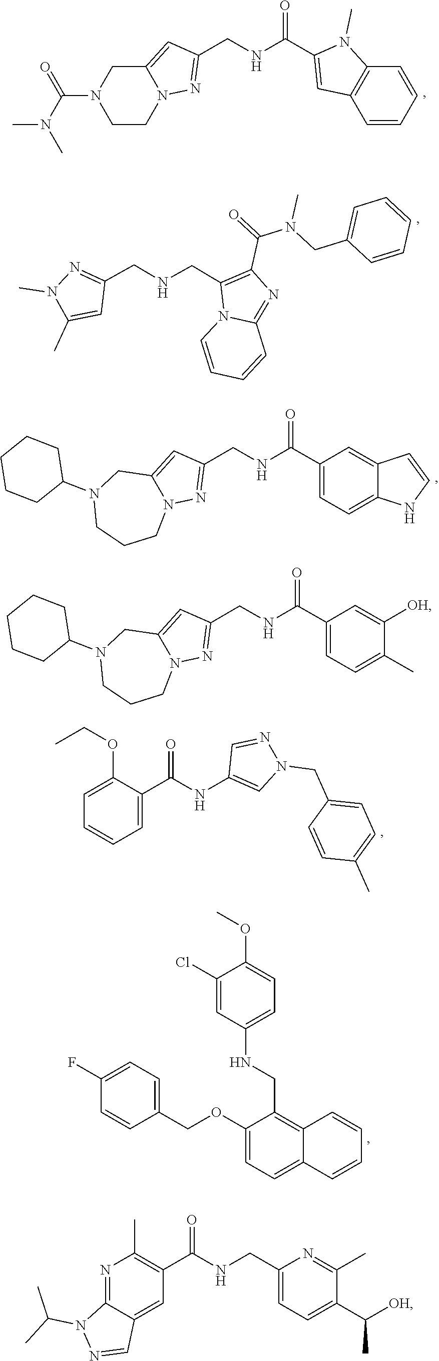

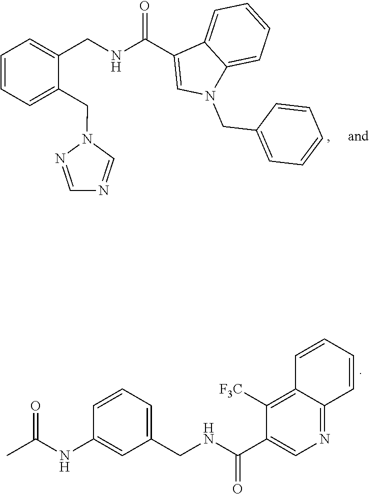 [SMILES: O=C(NCc1ccccc1Cn1cncn1)c1cn(Cc2ccccc2)c2ccccc12 ; CC(=O)Nc1cccc(CNC(=O)c2cnc3ccccc3c2C(F)(F)F)c1]

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A method of treating, preventing, or ameliorating a neurodegenerative disease, the method comprising administering to a subject in need thereof a therapeutically effective amount of at least one Tip60 activator or a pharmaceutically acceptable salt thereof.

2. The method of claim 1, wherein the neurodegenerative disease is at least one selected from the group consisting of Alzheimer's Disease, Parkinson's Disease, Huntington's Disease, Amyotrophic Lateral Sclerosis (ALS), Frontotemporal Dementia, Multiple System Atrophy, Progressive Supranuclear Palsy, Corticobasal Degeneration, Lewy Body Dementia, Friedreich's Ataxia, Spinocerebellar Ataxia, Spinal Muscular Atrophy, Prion Diseases (such as Creutzfeldt-Jakob Disease), Wernicke-Korsakoff Syndrome, Kennedy's Disease (Spinal Bulbar Muscular Atrophy), Wilson's Disease, Niemann-Pick Disease, and Gerstmann-Sträussler-Scheinker Syndrome (GSS).

3. The method of claim 1, wherein the neurodegenerative disease is at least one selected from the group consisting of Alzheimer's Disease, Parkinson's Disease, Huntington's Disease, Amyotrophic Lateral Sclerosis (ALS).

4. The method of claim 1, wherein the subject is further administered at least one additional agent suitable for the treatment, prevention, or amelioration of a neurodegenerative disease.

5. The method of claim 1, wherein the Tip60 activator is a compound of Formula (I) or a pharmaceutically acceptable salt thereof: 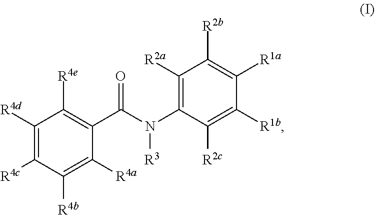
wherein:
R^{1a}, R^{1b}, and R^{1e} are each independently selected from the group consisting of H, optionally substituted C₁-C₆ alkyl, halogen, CN, and NO₂;
R^{1c} is halogen;
R^{1d} is C₁-C₃ haloalkyl;
R² is selected from the group consisting of H and optionally substituted C₁-C₆ alkyl;
R^{3a} and R^{3e} are each independently selected from the group consisting of optionally substituted C₁-C₁₂ alkoxy and optionally substituted C₁-C₂₀ alkyl; and
R^{3b}, R^{3c}, and R^{3d} are each independently selected from the group consisting of H, optionally substituted C₁-C₆ alkyl, halogen, CN, and NO₂.

6. The method of claim 5, wherein at least one of the following applies:
(a) R^{1a}, R^{1b}, and R^{1e} are each independently H;
(b) R^{1c} is Cl;
(c) R^{1d} is CF₃; and
(d) R² is H.

7. The method of claim 5, wherein R^{3c} is selected from the group consisting of OEt, OiPr,

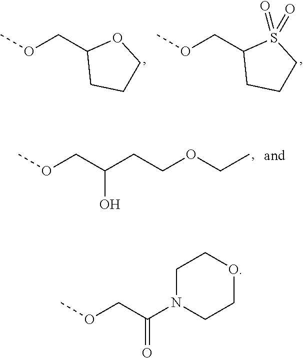

8. The method of claim 5, wherein R^{3c} is selected from the group consisting of H, (CH₂)₁₄CH₃, and O(CH₂)₂N(CH₃)₂.

9. The method of claim 5, wherein the compound of Formula (I) is selected from the group consisting of:

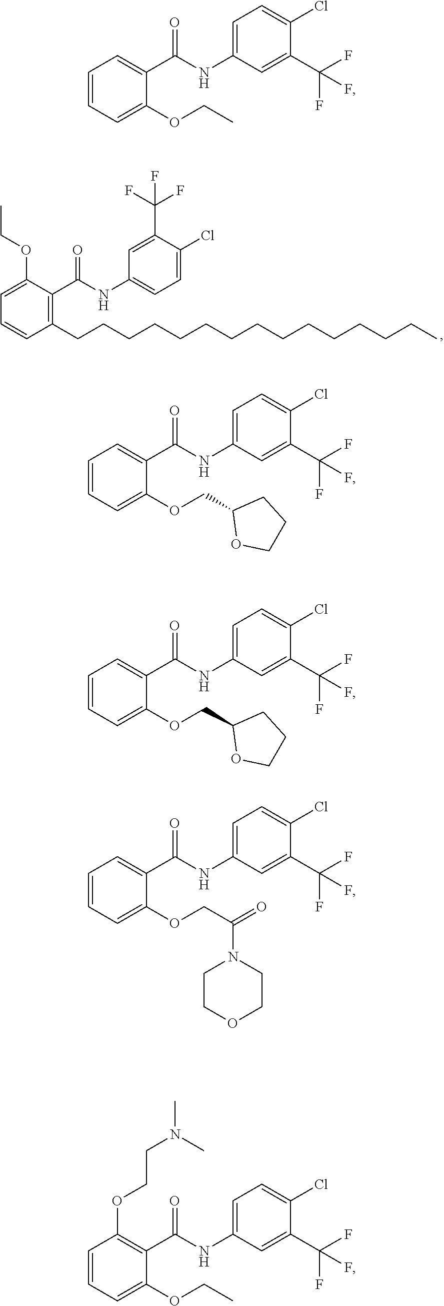

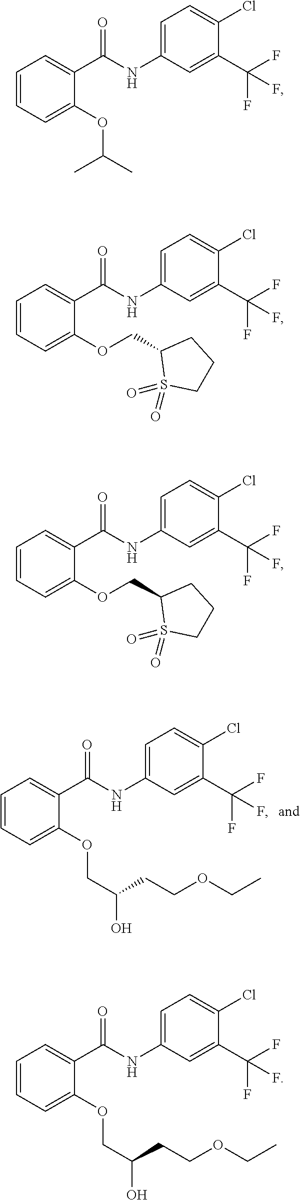 [SMILES: CC(C)Oc1ccccc1C(=O)Nc1ccc(Cl)c(C(F)(F)F)c1 ; O=C(Nc1ccc(Cl)c(C(F)(F)F)c1)c1ccccc1OC[C@@H]1CCCS1(=O)=O ; O=C(Nc1ccc(Cl)c(C(F)(F)F)c1)c1ccccc1OC[C@H]1CCCS1(=O)=O ; CCOCC[C@H](O)COc1ccccc1C(=O)Nc1ccc(Cl)c(C(F)(F)F)c1 ; CCOCC[C@@H](O)COc1ccccc1C(=O)Nc1ccc(Cl)c(C(F)(F)F)c1]

10. The method of claim 1, wherein the Tip60 activator is a compound of Formula (II) or a pharmaceutically acceptable salt thereof: 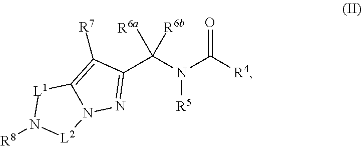
wherein:
R⁴ is selected from the group consisting of optionally substituted C₂-C₈ heteroaryl and C₆-C₁₀ aryl;
R⁵ is selected from the group consisting of H and optionally substituted C₁-C₆ alkyl;
R^{6a} and R^{6b} are each independently selected from the group consisting of H and optionally substituted C₁-C₆ alkyl;
R⁷ is selected from the group consisting of H, optionally substituted C₁-C₆ alkyl, halogen, CN, and NO₂;
R⁸ is selected from the group consisting of H, optionally substituted C₁-C₆ alkyl, optionally substituted C₃-C₈ cycloalkyl, optionally substituted C₂-C₈ heterocycloalkyl, and C(═O)N(R^{A})(R^{B});
L¹ is —[C(R^{9a})(R^{9b})]₁₋₂—;
L² is —[C(R^{10a})(R^{10b})]₁₋₂-;
each occurrence of R^{9a} and R^{9b} is independently selected from the group consisting of H and optionally substituted C₁-C₆ alkyl;
each occurrence of R^{10a} and R^{10b} is independently selected from the group consisting of H and optionally substituted C₁-C₆ alkyl; and
R^{A} and R^{B} are each independently selected from the group consisting of H and optionally substituted C₁-C₆ alkyl, or R^{A} and R^{B} combine with the N atom to which they are bound to form an optionally substituted C₂-C₈ heterocycloalkyl.

11. The method of claim 10, wherein at least one of the following applies:
(a) R⁴ is selected from the group consisting of 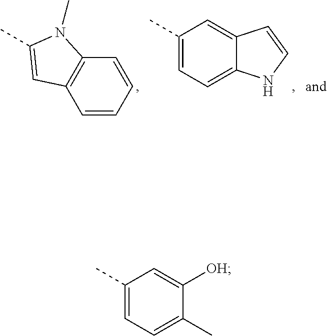
(b) R⁵ is H;
(c) R^{6a} and R^{6b} are each independently H;
(d) R⁷ is H;
(e) R⁸ is selected from the group consisting of cyclohexyl and C(═O)N(CH₃)₂;
(f) L¹ is —CH₂—;
(g) L² is selected from the group consisting of —(CH₂)₂— and —(CH₂)₃—.

12. The method of claim 10, wherein the compound of Formula (II) is selected from the group consisting of:

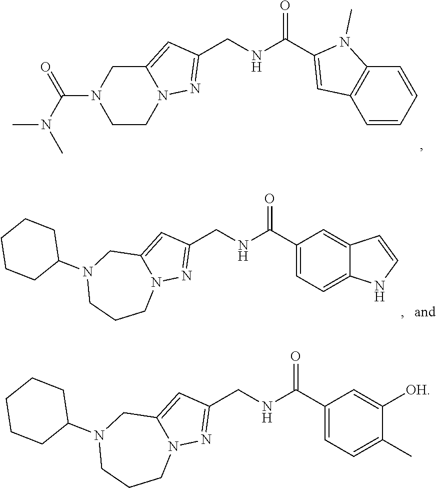 [SMILES: CN(C)C(=O)N1CCn2nc(CNC(=O)c3cc4ccccc4n3C)cc2C1 ; O=C(NCc1cc2n(n1)CCCN(C1CCCCC1)C2)c1ccc2[nH]ccc2c1 ; Cc1ccc(C(=O)NCc2cc3n(n2)CCCN(C2CCCCC2)C3)cc1O]

13. The method of claim 1, wherein the Tip60 activator is selected from the group consisting of:

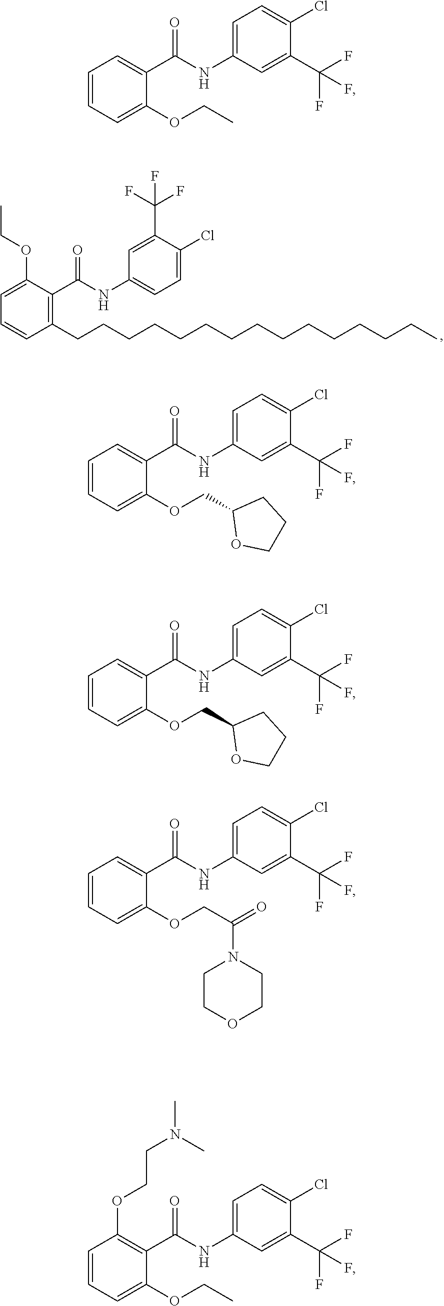

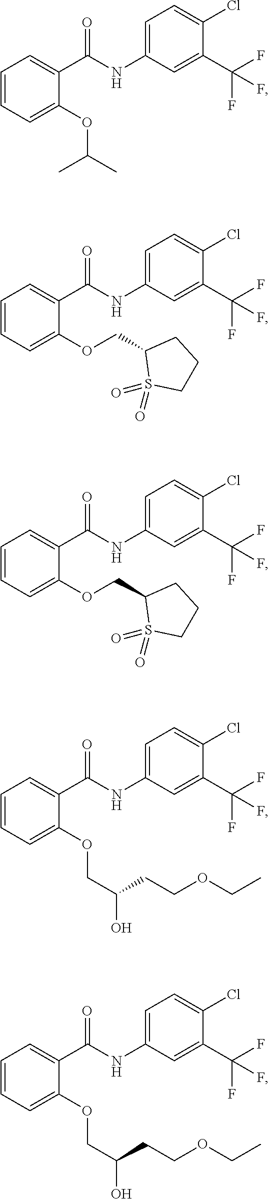 [SMILES: CC(C)Oc1ccccc1C(=O)Nc1ccc(Cl)c(C(F)(F)F)c1 ; O=C(Nc1ccc(Cl)c(C(F)(F)F)c1)c1ccccc1OC[C@@H]1CCCS1(=O)=O ; O=C(Nc1ccc(Cl)c(C(F)(F)F)c1)c1ccccc1OC[C@H]1CCCS1(=O)=O ; CCOCC[C@H](O)COc1ccccc1C(=O)Nc1ccc(Cl)c(C(F)(F)F)c1 ; CCOCC[C@@H](O)COc1ccccc1C(=O)Nc1ccc(Cl)c(C(F)(F)F)c1]

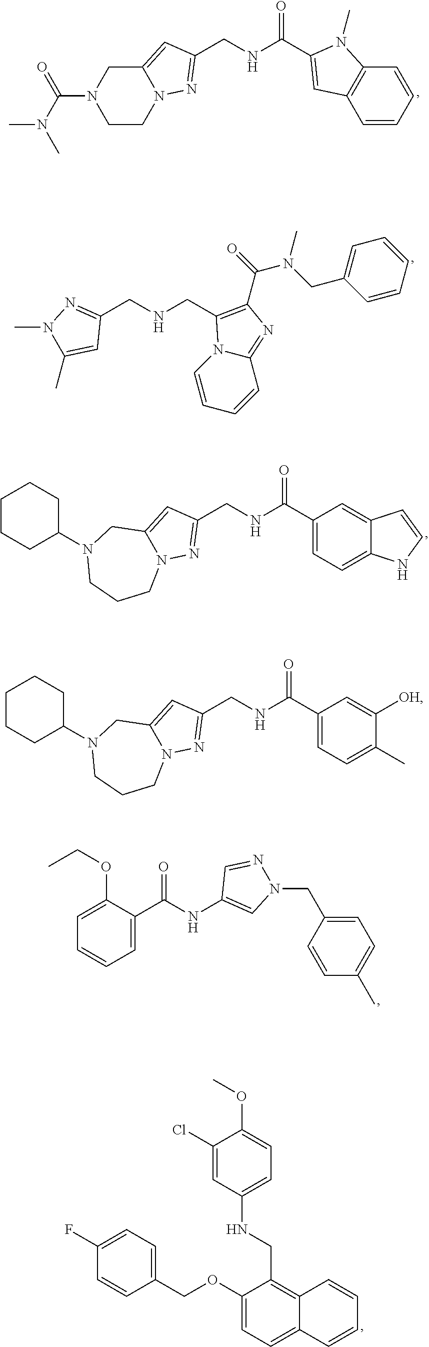

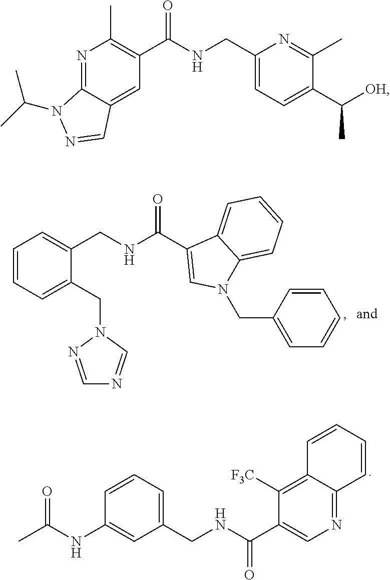 [SMILES: Cc1nc2c(cnn2C(C)C)cc1C(=O)NCc1ccc([C@H](C)O)c(C)n1 ; O=C(NCc1ccccc1Cn1cncn1)c1cn(Cc2ccccc2)c2ccccc12 ; CC(=O)Nc1cccc(CNC(=O)c2cnc3ccccc3c2C(F)(F)F)c1]

14. A method of treating, preventing, or ameliorating a Tip60-dependent cancer or Tip60-overexpressing cancer, the method comprising administering to a subject in need thereof a therapeutically effective amount of a Tip60 inhibitor or a pharmaceutically acceptable salt thereof.

15. The method of claim 14, wherein the Tip60 activator is a compound of Formula (I) or a pharmaceutically acceptable salt thereof: 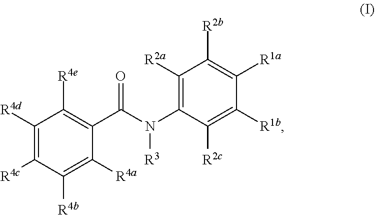
wherein:
R^{1a}, R^{1b}, and R^{1e} are each independently selected from the group consisting of H, optionally substituted C₁-C₆ alkyl, halogen, CN, and NO₂;
R^{1c} is halogen;
R^{1d} is C₁-C₃ haloalkyl;
R² is selected from the group consisting of H and optionally substituted C₁-C₆ alkyl;
R^{3a} and R^{3c} are each independently selected from the group consisting of optionally substituted C₁-C₁₂ alkoxy and optionally substituted C₁-C₂₀ alkyl; and
R^{3b}, R^{3c}, and R^{3d} are each independently selected from the group consisting of H, optionally substituted C₁-C₆ alkyl, halogen, CN, and NO₂.

16. The method of claim 14, wherein the Tip60 activator is a compound of Formula (II) or a pharmaceutically acceptable salt thereof: 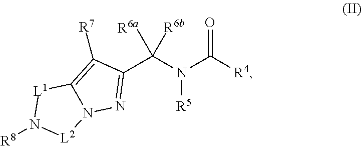
wherein:
R⁴ is selected from the group consisting of optionally substituted C₂-C₈ heteroaryl and C₆-C₁₀ aryl;
R⁵ is selected from the group consisting of H and optionally substituted C₁-C₆ alkyl;
R^{6c} and R^{6b} are each independently selected from the group consisting of H and optionally substituted C₁-C₆ alkyl;
R⁷ is selected from the group consisting of H, optionally substituted C₁-C₆ alkyl, halogen, CN, and NO₂;
R⁸ is selected from the group consisting of H, optionally substituted C₁-C₆ alkyl, optionally substituted C₃-C₈ cycloalkyl, optionally substituted C₂-C₈ heterocycloalkyl, and C(═O)N(R^{A})(R^{B});
L¹ is —[C(R^{9a})(R^{9b})]₁₋₂—;
L² is —[C(R^{10a})(R^{10b})]₁₋₂—;
each occurrence of R^{9c} and R^{9b} is independently selected from the group consisting of H and optionally substituted C₁-C₆ alkyl;
each occurrence of R¹⁰a and R^{10b} is independently selected from the group consisting of H and optionally substituted C₁-C₆ alkyl; and
R^{A} and R^{B} are each independently selected from the group consisting of H and optionally substituted C₁-C₆ alkyl, or R^{A} and R^{B} combine with the N atom to which they are bound to form an optionally substituted C₂-C₈ heterocycloalkyl.

17. The method of claim 14, wherein the Tip60 activator is selected from the group consisting of:

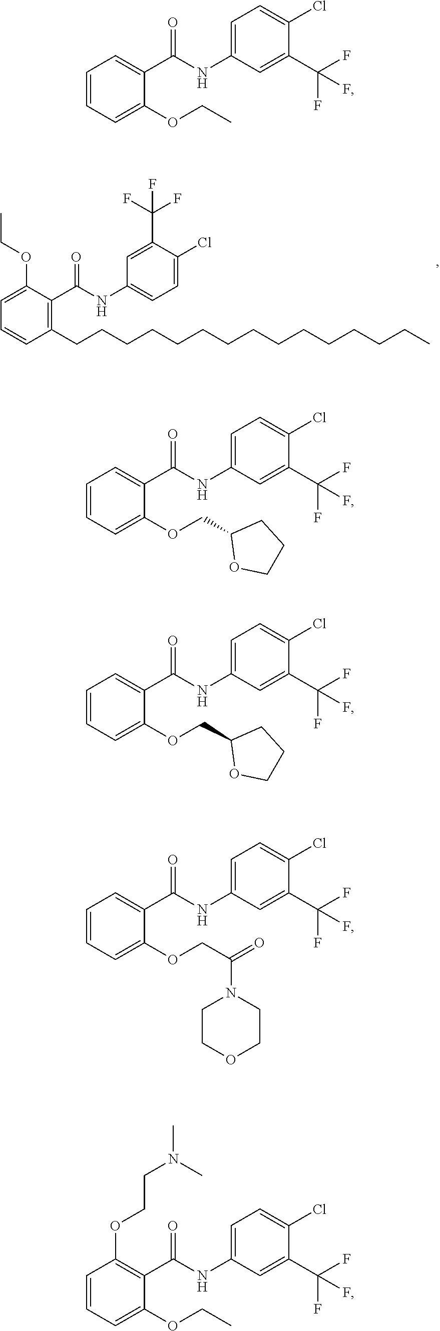 [SMILES: CCOc1ccccc1C(=O)Nc1ccc(Cl)c(C(F)(F)F)c1 ; CCCCCCCCCCCCCCCc1cccc(OCC)c1C(=O)Nc1ccc(Cl)c(C(F)(F)F)c1 ; O=C(Nc1ccc(Cl)c(C(F)(F)F)c1)c1ccccc1OC[C@@H]1CCCO1 ; O=C(Nc1ccc(Cl)c(C(F)(F)F)c1)c1ccccc1OC[C@H]1CCCO1 ; O=C(Nc1ccc(Cl)c(C(F)(F)F)c1)c1ccccc1OCC(=O)N1CCOCC1 ; CCOc1cccc(OCCN(C)C)c1C(=O)Nc1ccc(Cl)c(C(F)(F)F)c1]

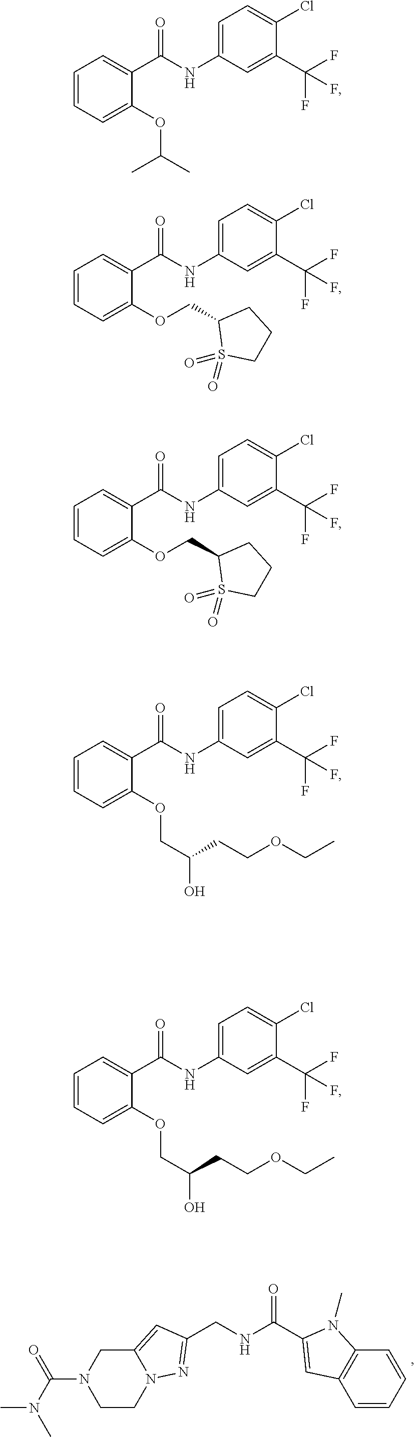

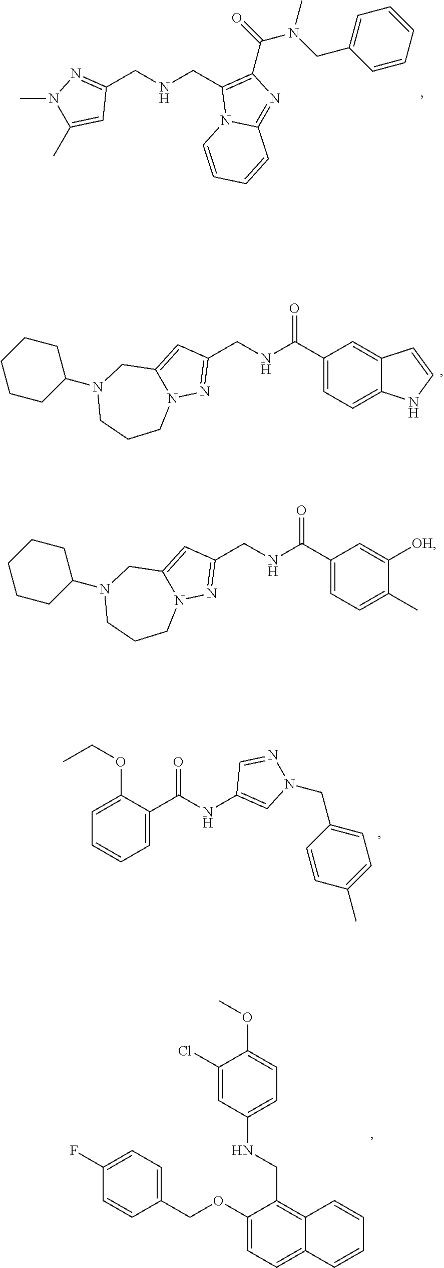 [SMILES: Cc1cc(CNCc2c(C(=O)N(C)Cc3ccccc3)nc3ccccn23)nn1C ; O=C(NCc1cc2n(n1)CCCN(C1CCCCC1)C2)c1ccc2[nH]ccc2c1 ; Cc1ccc(C(=O)NCc2cc3n(n2)CCCN(C2CCCCC2)C3)cc1O ; CCOc1ccccc1C(=O)Nc1cnn(Cc2ccc(C)cc2)c1 ; COc1ccc(NCc2c(OCc3ccc(F)cc3)ccc3ccccc23)cc1Cl]

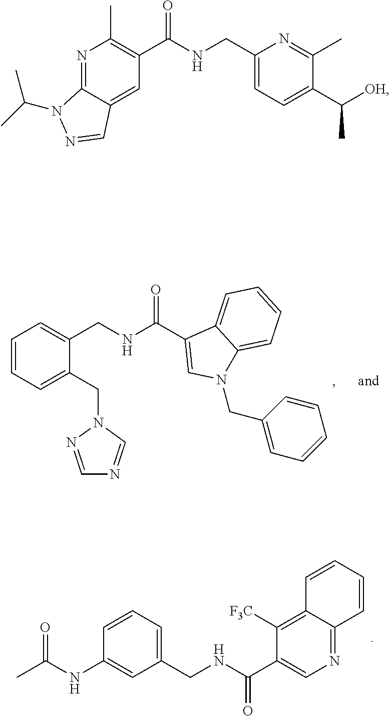 [SMILES: Cc1nc2c(cnn2C(C)C)cc1C(=O)NCc1ccc([C@H](C)O)c(C)n1 ; O=C(NCc1ccccc1Cn1cncn1)c1cn(Cc2ccccc2)c2ccccc12 ; CC(=O)Nc1cccc(CNC(=O)c2cnc3ccccc3c2C(F)(F)F)c1]

18. The method of claim 14, wherein the Tip60-dependent or Tip60-overexpressing is at least one selected from the group consisting of breast cancer, prostate cancer, colorectal cancer, lung cancer, ovarian cancer, glioblastoma, hepatocellular carcinoma, gastric cancer, leukemia, melanoma, pancreatic cancer, bladder cancer, cervical cancer, esophageal cancer, head and neck squamous cell carcinoma, renal cell carcinoma, thyroid cancer, non-small cell lung cancer, endometrial cancer, and lymphoma.

19. The method of claim 14, wherein the subject is further administered at least one additional agent suitable for the treatment, prevention, or amelioration of cancer.

20. A pharmaceutical composition comprising a pharmaceutically acceptable carrier and at least one compound selected from the group consisting of: 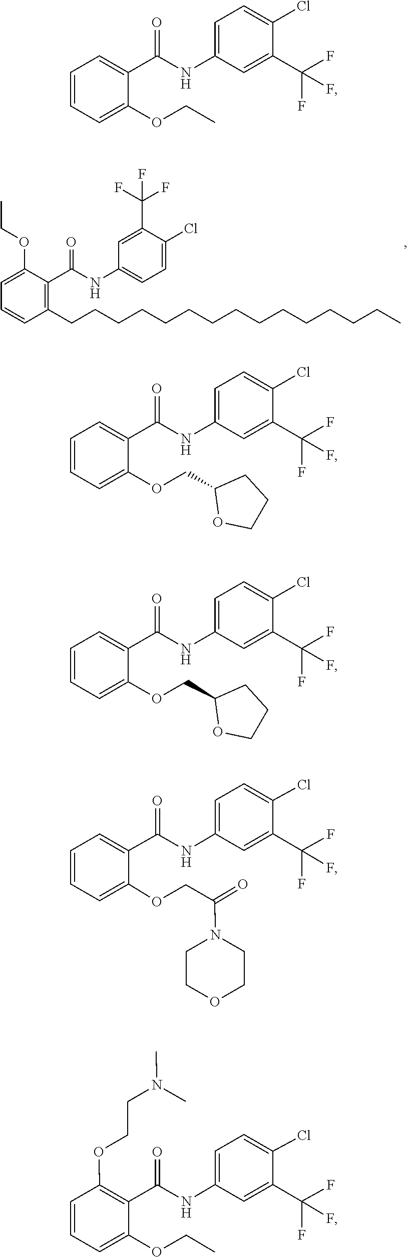 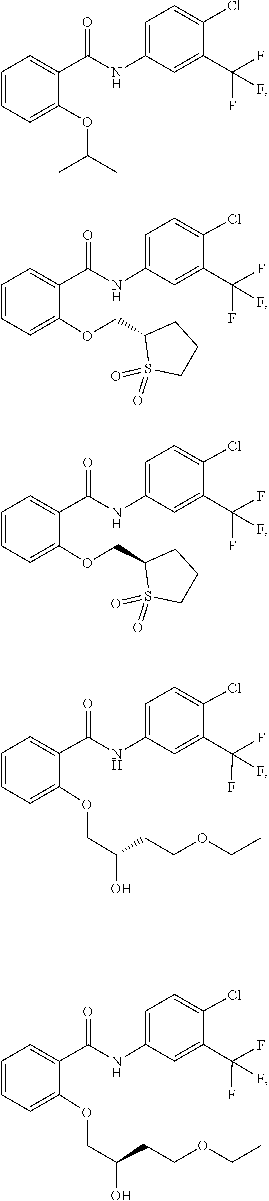 [SMILES: CC(C)Oc1ccccc1C(=O)Nc1ccc(Cl)c(C(F)(F)F)c1 ; O=C(Nc1ccc(Cl)c(C(F)(F)F)c1)c1ccccc1OC[C@@H]1CCCS1(=O)=O ; O=C(Nc1ccc(Cl)c(C(F)(F)F)c1)c1ccccc1OC[C@H]1CCCS1(=O)=O ; CCOCC[C@H](O)COc1ccccc1C(=O)Nc1ccc(Cl)c(C(F)(F)F)c1 ; CCOCC[C@@H](O)COc1ccccc1C(=O)Nc1ccc(Cl)c(C(F)(F)F)c1] 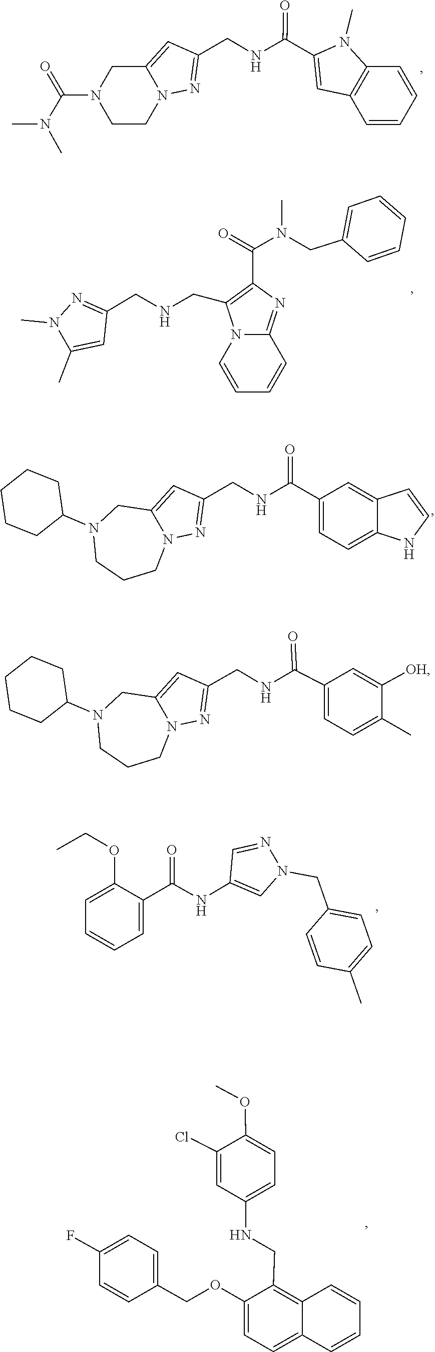 [SMILES: CN(C)C(=O)N1CCn2nc(CNC(=O)c3cc4ccccc4n3C)cc2C1 ; Cc1cc(CNCc2c(C(=O)N(C)Cc3ccccc3)nc3ccccn23)nn1C ; O=C(NCc1cc2n(n1)CCCN(C1CCCCC1)C2)c1ccc2[nH]ccc2c1 ; Cc1ccc(C(=O)NCc2cc3n(n2)CCCN(C2CCCCC2)C3)cc1O ; CCOc1ccccc1C(=O)Nc1cnn(Cc2ccc(C)cc2)c1 ; COc1ccc(NCc2c(OCc3ccc(F)cc3)ccc3ccccc23)cc1Cl] 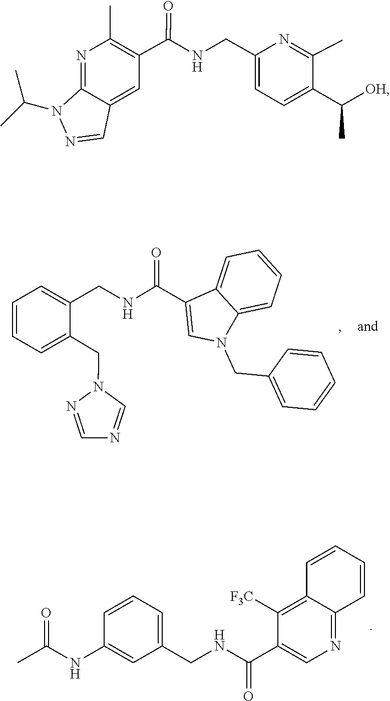 [SMILES: Cc1nc2c(cnn2C(C)C)cc1C(=O)NCc1ccc([C@H](C)O)c(C)n1 ; O=C(NCc1ccccc1Cn1cncn1)c1cn(Cc2ccccc2)c2ccccc12 ; CC(=O)Nc1cccc(CNC(=O)c2cnc3ccccc3c2C(F)(F)F)c1]
